SEQUENCE LISTING (170 sequences)

SEQ ID NO: 1 - DNA, 318 bp, Artificial Sequence
  Description of Artificial Sequence Synthetic polynucleotide
gacatccagatgacacagagccccagcacactgtctgccagcgtgggagacagagtgaccatcacatgcaagtgccagctgagcgtgggctacatgcactggtatcagcagaagcctggcaaggcccctaagctgctgatctacgacacaagcaagctggcctctggcgtgcccagcagattttctggctctggcagcggcaccgccttcacactgaccatctctagcctgcagcctgacgacttcgccacctactactgctttcaaggcagcggctaccctttcacctttggcggcggaacaaagctggaaatcaag

SEQ ID NO: 2 - DNA, 360 bp, Artificial Sequence
  Description of Artificial Sequence Synthetic polynucleotide
caagtgaccctgagagagtctggccctgctctggtcaagcccacacagaccctgacactgacctgcaccttcagcggctttagcctgagcacaagcggcatgagcgtcggctggattagacagcctcctggcaaagccctggaatggctggccgacatttggtgggacgacaagaaggactacaaccccagcctgaagtcccggctgaccatcagcaaagacaccagcgccaatcaggtggtgctgaaagtgaccaacatggaccctgccgacaccgccacctactactgtgccagatccatgatcaccaactggtacttcgacgtgtggggagccggcaccacagtgacagtttcttct

SEQ ID NO: 3 - DNA, 351 bp, Artificial Sequence
  Description of Artificial Sequence Synthetic polynucleotide
cagatgcagctggtgcagagcggcaccgaagtgaagaagcctggcgagagcctgaagatcagctgcaaaggcagcggctacggcttcatcacctactggatcggctgggtccgacagatgcctggcaaaggccttgagtggatgggcatcatctaccccggcgacagcgagacaagatacagccctagcttccagggccaagtgaccatcagcgccgacaagagcatcaacaccgcctacctgcagtggtccagcctgaaggcctctgacaccgccatctactattgtgccggcggaagcggcatcagcacccctatggatgtttggggccagggcaccaccgtgaccgtt

SEQ ID NO: 4 - DNA, 336 bp, Artificial Sequence
  Description of Artificial Sequence Synthetic polynucleotide
gacatccagctgacacagagccccgatagcctggccgtgtctctgggagaaagagccaccatcaactgcaagagcagccagagcgtgctgtactccagcatcaacaagaactacctggcctggtatcagcagaagcccggccagcctcctaagctgctgatctactgggccagcaccagagaaagcggcgtgcccgatagattttctggcagcggctctggcaccgacttcaccctgacaattagctccctgcaggccgaggatgtggccgtgtactactgccagcagtactacagcaccccttacacctttggccagggcaccaaggtggaaatc

SEQ ID NO: 5 - DNA, 345 bp, Artificial Sequence
  Description of Artificial Sequence Synthetic polynucleotide
gaggtgcagctggttgaatctggcggaggactggttcagcctggcggatctctgagactgtcttgtgccgccagcggcttcatcgtgtccagcaactacatgagctgggtccgacaggcccctggcaaaggacttgaatgggtgtccgtgatctacagcggcggcagcacctactacgccgattctgtgaagggcagattcaccatcagccggcacaacagcaagaacaccctgtacctgcagatgaacagcctgagagccgaggacaccgccgtgtactactgtgccagagaagcctacggcatggacgtgtggggacagggaacaaccgtgaccgttagctct

SEQ ID NO: 6 - DNA, 324 bp, Artificial Sequence
  Description of Artificial Sequence Synthetic polynucleotide
gacatcgtgatgactcagagccccagctttctgagcgccagcgtgggagacagagtgaccatcacatgtagagccagccagggcatcagcagctacctggcttggtatcagcagaagcctggcaaggcccctaaactgctgatctacgccgccagcacactgcagtctggcgtgccatctagattttccggcagcggctctggcaccgagttcaccctgaccatatctagcctgcagcctgaggacttcgccacctactactgccagcagctgaacagctaccctccttacacctttggccagggcaccaagctggaaatcaag

SEQ ID NO: 7 - DNA, 375 bp, Artificial Sequence
  Description of Artificial Sequence Synthetic polynucleotide
caggtgcagctggtgcagtctggggctgaagtgaagaagcctggggcctcagtgaaggtttcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagaaatgattactctagttcagggagtaaactactactacatggacgtctggggcaaagggaccacggtcaccgtctcctca

SEQ ID NO: 8 - DNA, 333 bp, Artificial Sequence
  Description of Artificial Sequence Synthetic polynucleotide
cagtctgccctgactcagcctgcctccgtgtctgggtcacctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactctcgtggtattcggcggagggaccaagctgaccgtccta

SEQ ID NO: 9 - DNA, 366 bp, Artificial Sequence
  Description of Artificial Sequence Synthetic polynucleotide
caggtgcagctgaaagagtctggacctggactggtggcccctagccagagcctgtctatcacctgtaccgtgtccggcttcctgctgatctccaatggcgtgcactgggtccgacagcctccaggcaaaggactggaatggctgggagtgatttgggctggcggcaacaccaactacaacagcgccctgatgagccgggtgtccatcagcaaggacaacagcaagagccaggtgttcctgaagatgaagtccctgcagaccgacgacaccgccatgtactactgcgccagagacttctacgactacgacgtgttctactacgccatggattactggggccagggcaccagcgtgacagtctcttct

SEQ ID NO: 10 - DNA, 327 bp, Artificial Sequence
  Description of Artificial Sequence Synthetic polynucleotide
caggccgtggtcacacaagaaagcgccctgacaacaagccctggcgagacagtgaccctgacctgcagatcttctacaggcgccgtgaccaccagcaactacgccaattgggtgcaagagaagcccgaccacctgttcacaggactgatcggcggcaccaacaatagagcacctggcgtgccagccagattcagcggatctctgatcggagacaaggccgcactgacaatcacaggcgcccagacagaggacgaggccatctacttttgcgccctgtggtacagcaaccactgggttttcggcggaggcaccaagctgacagttctg

SEQ ID NO: 11 - DNA, 366 bp, Artificial Sequence
  Description of Artificial Sequence Synthetic polynucleotide
caggttcagctgcaacagcctggcgccgaacttgttagacctggcgcctctgtgaagctgagctgtaaagccagcggctacaccctgaccacctactggatgaactggttcaagcagcggcccgatcagggcctcgagtggatcggaagaatcgacccctacgacagcgagacacactacaaccagaagttcaaggacaaggccatcctgaccgtggacagaagcagcagcacagcctacatgcagctgagcagcctgaccagcgaagatagcgccgtgtactactgcacccggtttctgcagatcaccaccatcatctacggcatggactactggggccagggcacaagcgtgacagtctcttct

SEQ ID NO: 12 - DNA, 333 bp, Artificial Sequence
  Description of Artificial Sequence Synthetic polynucleotide
gacgtggtcatgacacagaccccactgagcctgcctgtgtctctgggagatcaggccagcatcagctgcagatccagccagacactggtgcacagcaacggcaacacctacctgcactggtatctgcagaagcccggacagagccccaagctgctgatctacaaggtgtccaaccggttcagcggcgtgcccgatagattttctggcagcggctctggcaccgacttcaccctgaagatctccagagtggaagccgaggacctgggcgtgtacttctgcagccagaatacccacgtgccatacacctttggcggaggcaccaagctggaaatc

SEQ ID NO: 13 - DNA, 363 bp, Artificial Sequence
  Description of Artificial Sequence Synthetic polynucleotide
caggttcagctggttcagtctggcgccgaagtgaagaaacctggcagcagcgtgaaggtgtcctgcaagtctagcggcggcaccagcaacaactacgccatctcttgggtccgacaggcccctggacaaggcttggattggatgggcggcatcagccctatcttcggcagcacagcctacgctcagaaattccagggcagagtgaccatcagcgccgacatcttcagcaacaccgcctacatggaactgaacagcctgaccagcgaggataccgccgtgtacttctgtgccagacacggcaattactactactacagcggcatggacgtgtggggccagggaacaacagtgaccgttagctct

SEQ ID NO: 14 - DNA, 330 bp, Artificial Sequence
  Description of Artificial Sequence Synthetic polynucleotide
cagtctgctctgacacagcctccagccgtgtctggaacacctggccagagagtgaccatcagctgtagcggcagcgacagcaatatcggaaggcggagcgtgaactggtatcagcagttccctggcacagcccctaagctgctgatctacagcaacgaccagcggcctagcgtggtgcccgatagattttctggcagcaagagcggcacaagcgccagcctggctatttctggactgcagagcgaggacgaggccgagtattattgtgccgcctgggacgattctctgaagggcgctgtttttggcggcggaacccagctgacagttctg

SEQ ID NO: 15 - DNA, 390 bp, Artificial Sequence
  Description of Artificial Sequence Synthetic polynucleotide
agcgaagtgcagctggtggagtctggggctgaggtgaagaagcctggggcctcagtgaaggtttcctgcaaggtttccggatacaccctcactgaattatccatgcactgggtgcgacaggctcctggaaaagggcttgagtggatgggaggttttgatcctgaagatggtgaaacaatgtacgcacagaagttccagggcagagtcaccatgaccgaggacacatctacagacacagcctacatggagctgagcagcctgagatctgaggacacggccgtgtattactgtgcaacctcgacagcagtggctggcacacctgacctcttcgactactactacggtatggacgtctggggccaagggaccacggtcaccgtctcctcagcc

SEQ ID NO: 16 - DNA, 339 bp, Artificial Sequence
  Description of Artificial Sequence Synthetic polynucleotide
tccgaaatagtgatgacgcagtctccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcacgcaagctacacaatttccgtacacttttggccaggggaccaaagtggatatcaag

SEQ ID NO: 17 - DNA, 375 bp, Artificial Sequence; identical to SEQ ID NO: 7
  Description of Artificial Sequence Synthetic polynucleotide

SEQ ID NO: 18 - DNA, 333 bp, Artificial Sequence; identical to SEQ ID NO: 8
  Description of Artificial Sequence Synthetic polynucleotide

SEQ ID NO: 19 - protein, 8 aa, Artificial Sequence
  Description of Artificial Sequence Synthetic peptide
GYTFTKYW

SEQ ID NO: 20 - protein, 8 aa, Artificial Sequence
  Description of Artificial Sequence Synthetic peptide
INPSTGYS

SEQ ID NO: 21 - protein, 13 aa, Artificial Sequence
  Description of Artificial Sequence Synthetic peptide
VRGYDSHYYVMDY

SEQ ID NO: 22 - protein, 10 aa, Artificial Sequence
  Description of Artificial Sequence Synthetic peptide
ESVEYYGTTL

SEQ ID NO: 23 - protein, 3 aa, Artificial Sequence
  Description of Artificial Sequence Synthetic peptide
GAS

SEQ ID NO: 24 - protein, 9 aa, Artificial Sequence
  Description of Artificial Sequence Synthetic peptide
QQSRKVPYT

SEQ ID NO: 25 - protein, 120 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSS

SEQ ID NO: 26 - protein, 111 aa, Artificial Sequence
  Description of Artificial Sequence Synthetic polypeptide
DIVITQDTASLAVSRGQRATISCRASESVEYYGTTLMQWYQQRPGQPPKLLIYGASNVESGVPARFSGSGSGTDFSLNIHPVEEDDIAMYFCQQSRKVPYTFGGGTKLEIK

SEQ ID NO: 27 - protein, 98 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 28 - protein, 113 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAK

SEQ ID NO: 29 - protein, 107 aa, Artificial Sequence
  Description of Artificial Sequence Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 12 aa, Artificial Sequence
  Description of Artificial Sequence Synthetic peptide
EPKSCDKTHTCP

SEQ ID NO: 31 - protein, 107 aa, Artificial Sequence [sequence omitted]

SEQ ID NO: 32 - protein, 5 aa, Artificial Sequence
  Description of Artificial Sequence Synthetic peptide
ASTKG

SEQ ID NO: 33 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVLHEALHSHYTQKSLSLSPGK

SEQ ID NO: 34 - protein, 220 aa, Artificial Sequence
  Description of Artificial Sequence Synthetic polypeptide
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 220 aa, Artificial Sequence
  Description of Artificial Sequence Synthetic polypeptide
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 36 - protein, 220 aa, Artificial Sequence
  Description of Artificial Sequence Synthetic polypeptide
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 219 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 39 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 41 - protein, 220 aa, Artificial Sequence
  Description of Artificial Sequence Synthetic polypeptide
PCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 42 - protein, 220 aa, Artificial Sequence
  Description of Artificial Sequence Synthetic polypeptide
PCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 43 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 45 - protein, 220 aa, Artificial Sequence
  Description of Artificial Sequence Synthetic polypeptide
PCPAPELLGGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 220 aa, Artificial Sequence
  Description of Artificial Sequence Synthetic polypeptide
PCPAPELLGGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 47 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 221 aa, Artificial Sequence
  Description of Artificial Sequence Synthetic polypeptide
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 49 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 50 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVLHGALHSHYTQKYLSLSPGK

SEQ ID NO: 51 - protein, 220 aa, Artificial Sequence
  Description of Artificial Sequence Synthetic polypeptide
PCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 220 aa, Artificial Sequence
  Description of Artificial Sequence Synthetic polypeptide
PCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 53 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 55 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 220 aa, Artificial Sequence
  Description of Artificial Sequence Synthetic polypeptide
PCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 220 aa, Artificial Sequence; identical to SEQ ID NO: 57
  Description of Artificial Sequence Synthetic polypeptide

SEQ ID NO: 60 - protein, 220 aa, Artificial Sequence
  Description of Artificial Sequence Synthetic polypeptide
PCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 220 aa, Artificial Sequence
  Description of Artificial Sequence Synthetic polypeptide
PCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 62 - protein, 220 aa, Artificial Sequence
  Description of Artificial Sequence Synthetic polypeptide
PCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 220 aa, Artificial Sequence
  Description of Artificial Sequence Synthetic polypeptide
PCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 64 - protein, 220 aa, Artificial Sequence
  Description of Artificial Sequence Synthetic polypeptide
PCPAPELLGGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 220 aa, Artificial Sequence
  Description of Artificial Sequence Synthetic polypeptide
PCPAPELLGGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 66 - protein, 220 aa, Artificial Sequence
  Description of Artificial Sequence Synthetic polypeptide
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 220 aa, Artificial Sequence
  Description of Artificial Sequence Synthetic polypeptide
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 68 - protein, 220 aa, Artificial Sequence
  Description of Artificial Sequence Synthetic polypeptide
PCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 220 aa, Artificial Sequence
  Description of Artificial Sequence Synthetic polypeptide
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 70 - protein, 220 aa, Artificial Sequence
  Description of Artificial Sequence Synthetic polypeptide
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 220 aa, Artificial Sequence; identical to SEQ ID NO: 69
  Description of Artificial Sequence Synthetic polypeptide

SEQ ID NO: 72 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 74 - protein, 220 aa, Artificial Sequence
  Description of Artificial Sequence Synthetic polypeptide
PCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 220 aa, Artificial Sequence
  Description of Artificial Sequence Synthetic polypeptide
PCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 76 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 77 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 78 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 79 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - protein, 220 aa, Artificial Sequence
  Description of Artificial Sequence Synthetic polypeptide
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 220 aa, Artificial Sequence
  Description of Artificial Sequence Synthetic polypeptide
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 82 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSVIAVEWESNGQPENNYKTTPPVLDSDGSFFLYSKLTVDKSRWQQGNVFSCSVMHEALHHHYTQKSLSLSPGK

SEQ ID NO: 83 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 84 - protein, 220 aa, Artificial Sequence
  Description of Artificial Sequence Synthetic polypeptide
PCPAPELLGGPSVFLFPPKPKDTLMISRTIEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHHHYTQKSLSLSPGK

SEQ ID NO: 85 - protein, 220 aa, Artificial Sequence
  Description of Artificial Sequence Synthetic polypeptide
PCPAPELLGGPSVFLFPPKPKDTLMISRTIEVTCVVVDVSHEDPEVKFNWYVDGVEVHNAKTKPREEQYNSTYRVVSVLTVLHIDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 220 aa, Artificial Sequence
  Description of Artificial Sequence Synthetic polypeptide
PCPAPELLGGPSVFLFPPKPKDTLMISRTIEVTCVVVDVSHEDPEVKFNWYVDGVEVHNAKTKPREEQYNSTYRVVSVLTVLHIDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 87 - protein, 220 aa, Artificial Sequence
  Description of Artificial Sequence Synthetic polypeptide
PCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 88 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVLHEALHSHYTQKSLSLSPGK

SEQ ID NO: 89 - protein, 220 aa, Artificial Sequence
  Description of Artificial Sequence Synthetic polypeptide
PCPAPELLGGPSVFLFPPKPKDTLMISRTIEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 90 - protein, 220 aa, Artificial Sequence
  Description of Artificial Sequence Synthetic polypeptide
PCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 91 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 92 - protein, 220 aa, Artificial Sequence
  Description of Artificial Sequence Synthetic polypeptide
PCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 220 aa, Artificial Sequence
  Description of Artificial Sequence Synthetic polypeptide
PCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 94 - protein, 220 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSVIAVEWESNGQPENNYKTTPPVLDSDGSFFLYSKLTVDKSRWQQGNVFSCSVLHEALHSHYTQKSLSLSPGK

SEQ ID NO: 95 - protein, 220 aa, Artificial Sequence; identical to SEQ ID NO: 33
  Description of Artificial Sequence Synthetic polypeptide

SEQ ID NO: 96 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSVIAVEWESNGQPENNYKTTPPVLDSDGSFFLYSKLTVDKSRWQQGNVFSCSVMHEALHHHYTQKSLSLSPGK

SEQ ID NO: 98 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 101 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 107 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 109 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 110 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTIEVTCVVVDVSHEDPEVKFNWYVDGVEVHNAKTKPREEQYNSTYRVVSVLTVLHIDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 112 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 113 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVLHEALHSHYTQKSLSLSPGK

SEQ ID NO: 120 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSVIAVEWESNGQPENNYKTTPPVLDSDGSFFLYSKLTVDKSRWQQGNVFSCSVLHEALHSHYTQKSLSLSPGK

SEQ ID NO: 121 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 122 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 123 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 124 - protein, 450 aa, Artificial Sequence; identical to SEQ ID NO: 123
  Description of Artificial Sequence Synthetic polypeptide

SEQ ID NO: 125 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 126 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 127 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 128 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVLHGALHSHYTQKYLSLSPGK

SEQ ID NO: 129 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 130 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 131 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 133 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 135 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 136 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 141 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 142 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 143 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 145 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVLHEALHSHYTQKSLSLSPGK

SEQ ID NO: 146 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 148 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 150 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 151 - protein, 450 aa, Artificial Sequence; identical to SEQ ID NO: 137
  Description of Artificial Sequence Synthetic polypeptide

SEQ ID NO: 152 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 153 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 155 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 156 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTIEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHHHYTQKSLSLSPGK

SEQ ID NO: 157 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTIEVTCVVVDVSHEDPEVKFNWYVDGVEVHNAKTKPREEQYNSTYRVVSVLTVLHIDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 158 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTIEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 159 - protein, 450 aa, Artificial Sequence
  Description of Artificial Sequence Synthetic polypeptide
QVQFQQSGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 160 - protein, 218 aa, Artificial Sequence
  Description of Artificial Sequence Synthetic polypeptide
DIVITQDTASLAVSRGQRATISCRASESVEYYGTTLMQWYQQRPGQPPKLLIYGASNVESGVPARFSGSGSGTDFSLNIHPVEEDDIAMYFCQQSRKVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - DNA, 26 bp, Artificial Sequence
  Description of Artificial Sequence Synthetic oligonucleotide
gtcctagctcttcacggtctcccagc

SEQ ID NO: 162 - DNA, 27 bp, Artificial Sequence
  Description of Artificial Sequence Synthetic oligonucleotide
gccatgagaccttcgaagagcgtcgta

SEQ ID NO: 163 - DNA, 22 bp, Artificial Sequence
  Description of Artificial Sequence Synthetic oligonucleotide
gtcctggctcttcacggtctcc

SEQ ID NO: 164 - DNA, 27 bp, Artificial Sequence
  Description of Artificial Sequence Synthetic oligonucleotide
cggatgagaccttcgaagagcgacgtc

SEQ ID NO: 165 - DNA, 26 bp, Artificial Sequence
  Description of Artificial Sequence Synthetic oligonucleotide
gctctagctcttcacggtctccctcc

SEQ ID NO: 166 - DNA, 27 bp, Artificial Sequence
  Description of Artificial Sequence Synthetic oligonucleotide
aagatgagaccttcgaagagctaggca

SEQ ID NO: 167 - DNA, 26 bp, Artificial Sequence
  Description of Artificial Sequence Synthetic oligonucleotide
gctctagctcttcacggtctccctcc

SEQ ID NO: 168 - DNA, 27 bp, Artificial Sequence
  Description of Artificial Sequence Synthetic oligonucleotide
ggtctgagaccttcgaagagctcgctg

SEQ ID NO: 169 - DNA, 22 bp, Artificial Sequence
  Description of Artificial Sequence Synthetic oligonucleotide
gctctagctcttcacggtctcc

SEQ ID NO: 170 - DNA, 27 bp, Artificial Sequence
  Description of Artificial Sequence Synthetic oligonucleotide
tcagtgagaccttcgaagagctaggca

(19) United States
(12) Patent Application Publication
(10) Pub. No.: US 2024/0109954 A1
(43) Pub. Date: Apr. 4, 2024

(54) FC-ENHANCED ANTIBODIES FOR PREVENTION AND TREATMENT OF EBOLA VIRUS INFECTION

(71) Applicant: The General Hospital Corporation, Boston, MA (US)

(72) Inventors: Galit Alter, Winchester, MA (US); Richard Lu, Somerville, MA (US)

(21) Appl. No.: 18/274,641

(22) PCT Filed: Feb. 1, 2022
(86) PCT No.: PCT/US2022/014779

§ 371 Date: Jul. 27, 2023

Related U.S. Application Data
(60) Provisional application No. 63/144,383, filed on Feb. 1, 2021.

(51) Int. Cl. C07K 16/10; A61P 31/14

(52) U.S. Cl. CPC C07K 16/10; A61P 31/14; C07K 2317/14; C07K 2317/524; C07K 2317/526; C07K 2317/55; C07K 2317/565

(57) ABSTRACT

Described herein are Fe-enhanced antibodies and methods of use thereof. Also described are Fe-enhanced antibodies to treat Ebola virus infection.

### CROSS-REFERENCE TO RELATED APPLICATION

This application claims the benefit of U.S. Provisional Application No. 63/144,383, filed on Feb. 1, 2021. The entire contents of the foregoing are incorporated herein by reference.

### STATEMENT OF FEDERALLY SPONSORED RESEARCH

This invention was made with government support under Grant No. HR0011-18-2-0001 awarded by Defense Advanced Research Projects Agency (DARPA). The government has certain rights in the invention.

### BACKGROUND

Effective methods to produce protective versus immunopathological antibodies against many transmissible and proliferative diseases do not currently exist. Ebola virus (EBOV) cause severe viral hemorrhagic fever in humans and non-human primates, with a fatality rate of up to about 90% in human outbreaks. (Murin, C D. et al., (2014), Proc Natl Acad Sci USA, 111(48): 17182-17187). Protective EBOV antibodies have neutralizing activity and induction of antibody constant domain (Fc)-mediated innate immune effector functions (Gunn B. M. et al, 2021, Immunity 54, 815-828). However, the precise effector functions that track with protection have yet to be defined. Accordingly, there is still a need in the art to identify the most effective antibodies, which can be used to prevent or treat an Ebola virus infection.

### SUMMARY

This disclosure is based on the findings, inter alia, that certain engineered Fc domain variants of the Ebola VIC16 antibody can be used to treat Ebola-infected mice. In particular, the inventors applied a high-throughput platform for engineering the antibody Fc domains to define protective profiles. Through this platform, the inventors surprisingly discovered that Fc variants with high complement deposition and moderate NK cell activity completely protected infected mice from disease.

Other features and advantages of the invention will be apparent from the Detailed Description, and from the claims. Thus, other aspects of the invention are described in the following disclosure and are within the ambit of the invention.

In one aspect, the disclosure features an antibody comprising an Fab binding domain that binds to the Ebola virus glycoprotein, and an Fc domain comprising constant heavy (CH)2 and CH3 domains, wherein the Fab binding domain comprises (a) a heavy chain variable region (VH) comprising a VH-complementarity determining region (CDR)1, a VH-CDR2, and a VH-CDR3 from the amino acid sequence of SEQ ID NO:25; and (b) a light chain variable region (VL) comprising a VL-CDR1, a VL-CDR2, and a VL-CDR3 from the amino acid sequence of SEQ ID NO:26; and wherein the Fc domain comprises an amino acid sequence with at least 80%, 85%, 90%, 95%, 99%, or 100% sequence identity to the amino acid sequence set forth in any one of SEQ ID NOs: 35-95.

In some embodiments, the VH comprises the VH-CDR1 comprising the amino acid sequence of SEQ ID NO: 19, the VH-CDR2 comprising the amino acid sequence of SEQ ID NO: 20, and the VH-CDR3 comprising the amino acid sequence of SEQ ID NO: 21; and the VL comprises the VL-CDR1 comprising the amino acid sequence of SEQ ID NO: 22, the VL-CDR2 comprising the amino acid sequence of SEQ ID NO: 23, and the VL-CDR3 comprising the amino acid sequence of SEQ ID NO: 24.

In some embodiments, the antibody comprises a heavy chain (HC) comprising the amino acid sequence set forth in any one of SEQ ID NOs: 99, 131, and 139 and a light chain comprising the amino acid sequence set forth in SEQ ID NO: 160.

In some embodiments, the antibody comprises a constant heavy (CH) chain comprising the amino acid sequence set forth in any one of SEQ ID NOs: 35-95, wherein the constant heavy chain comprises CH2 and CH3 domains.

In some embodiments, the antibody comprises a heavy chain (HC) comprising the amino acid sequence set forth in any one of SEQ ID NOs: 97-104 and 106-159 and a light chain comprising the amino acid sequence set forth in SEQ ID NO: 160.

In some embodiments, the disclosure features a composition comprising the antibody of the disclosure and a pharmaceutically acceptable carrier. In some embodiments, the disclosure features an isolated polynucleotide or polynucleotides encoding the antibody. In some embodiments, the disclosure features a vector or vectors comprising the polynucleotide or polynucleotides encoding the antibody. In some embodiments, the disclosure features an isolated cell comprising the polynucleotide or polynucleotides encoding the antibody.

In another aspect, the disclosure features a method of making an antibody that specifically binds to Ebola virus, the method comprising: (a) culturing the cell of disclosed herein under conditions that result in the expression of the antibody, and (b) isolating the antibody.

In yet another aspect, the disclosure features a method of enhancing at least one of the following in a subject in need thereof: (a) complement deposition; (b) cellular phagocytosis; and (c) NK cell activation; the method comprising administering to the subject, the antibody disclosed herein.

In another aspect, the disclosure features a method for treating Ebola virus infection comprising administering to a subject in need thereof a composition comprising an effective amount of an isolated monoclonal antibody, wherein the monoclonal antibody has an Fab binding domain that binds to the Ebola virus glycoprotein, and an Fc domain comprising constant heavy (CH)2 and CH3 domains; wherein the Fab binding domain comprises (a) a heavy chain variable region (VH) comprising a VH-complementarity determining region (CDR)1, a VH-CDR2, and a VH-CDR3 from the amino acid sequence of SEQ ID NO:25; and (b) a light chain variable region (VL) comprising a VL-CDR1, a VL-CDR2, and a VL-CDR3 from the amino acid sequence of SEQ ID NO:26; and and wherein the Fc domain comprises an amino acid sequence with at least 80%, 85%, 90%, 95%, 99%, or 100% sequence identity to the amino acid sequence set forth in any one of SEQ ID NOs: 35-95.

In some embodiments of the above methods, the VH comprises the VH-CDR1 comprising the amino acid sequence of SEQ ID NO: 19, the VH-CDR2 comprising the amino acid sequence of SEQ ID NO: 20, and the VH-CDR3 comprising the amino acid sequence of SEQ ID NO: 21; and the VL comprises the VL-CDR1 comprising the amino acid sequence of SEQ ID NO: 22, the VL-CDR2 comprising the amino acid sequence of SEQ ID NO: 23, and the VL-CDR3 comprising the amino acid sequence of SEQ ID NO: 24.

In some embodiments of the above methods, the antibody comprises a constant heavy (CH) chain with the amino acid sequence set forth in any one of SEQ ID NOs: 35-95, wherein the constant heavy chain comprises CH2, and CH3 domains.

In some embodiments of the above methods, the antibody comprises a heavy chain (HC) comprising the amino acid sequence set forth in any one of SEQ ID NOs: 97-104 and 106-159 and a light chain comprising the amino acid sequence set forth in SEQ ID NO: 160. In some embodiments of the above methods, the antibody comprises a heavy chain (HC) comprising the amino acid sequence set forth in any one of SEQ ID NOs: 99, 131, and 139 and a light chain comprising the amino acid sequence set forth in SEQ ID NO: 160.

In some embodiments of the above methods, the monoclonal antibody is an IgG1 antibody.

In some embodiments of the above methods, the further comprising administering a therapeutic agent. In some embodiments, the therapeutic agent is one or more of interferon alpha, atoltivimab, maftivimab, odesivimab-ebgn, and ansuvimab-zykl.

In some embodiments, the disclosure features the use of the antibody disclosed herein in the manufacture of a medicament for treatment of an Ebola virus infection. In some aspects, the disclosure features a method for producing a monoclonal antibody with a profile of functional activity directed against a pathogen of interest (“a functional profile”); said method comprising the steps of: a) generating a library of IgG1 Fc domains, each comprising a different Fc mutation, thereby generating Fc variants; and b) generating plasmids encoding each of the Fc variants linked to an Fab binding domain, wherein the Fab binding domain comprises variable heavy and light chains of an antibody that is reactive against the pathogen of interest, thereby forming a Fab-Fc variant; and c) expressing the Fab-Fc variants from the plasmids; and d) determining the functional profile of each Fab-Fc variant, thereby producing a monoclonal antibody with a profile of functional activity directed against a pathogen of interest.

In some embodiments, the functional profile comprises determining the level of at least one of phagocytosis of monocytes and/or neutrophils; complement deposition; NK cell degranulation; NK cell secretion of cytokine IFNγ and chemokine MIP-1β and expression of membrane protein CD107a; neutralizing activity; and FcyR binding.

In some embodiments, the pathogen of interest is Ebola virus. In some embodiments, the antibody that is reactive against Ebola virus is VIC16.

In some embodiments, the method produces the VIC16 antibody comprising a heavy chain variable region (VH) comprising a VH-complementarity determining region (CDR)1, a VH-CDR2, and a VH-CDR3 from the amino acid sequence of SEQ ID NO:25; and a light chain variable region (VL) comprising a VL-CDR1, a VL-CDR2, and a VL-CDR3 from the amino acid sequence of SEQ ID NO:26.

In some embodiments, the VH comprises the VH-CDR1 comprising the amino acid sequence of SEQ ID NO:19, the VH-CDR2 comprising the amino acid sequence of SEQ ID NO:20, and the VH-CDR3 comprising the amino acid sequence of SEQ ID NO:21; and the VL comprises the VL-CDR1 comprising the amino acid sequence of SEQ ID NO:22, the VL-CDR2 comprising the amino acid sequence of SEQ ID NO:23, and the VL-CDR3 comprising the amino acid sequence of SEQ ID NO:24.

In some embodiments, the method produces the VIC16 antibody comprising a constant heavy (CH) chain comprising the amino acid sequence set forth in any one of SEQ ID NOs: 35-95, wherein the CH chain comprises CH2 and CH3 domains. In some embodiments, the method produces the VIC16 antibody comprising a CH chain with least 80%, 85%, 90%, 95%, 99%, or 100% sequence identity to the amino acid sequence set forth in any one of SEQ ID NOs: 35-95, wherein the CH chain comprises CH2, and CH3 domains.

In some embodiments, the method produces the VIC16 antibody comprising a heavy chain comprising the amino acid sequence set forth in any one of SEQ ID NOs: 97-104, and 106-159 and a light chain comprising the amino acid sequence set forth in SEQ ID NO: 160.

In some embodiments, the Fab-Fc variant is selected from Table 6 or from an amino acid sequence that is at least 80% to 99% identical to the Fab-Fc variant selected from Table 6.

In some embodiments, the Fc variants are selected from Table 2 or from amino acid sequences that are at least 80% to 99% identical to the Fc variants selected from Table 2.

In some embodiments, the Fab binding domain is selected from SEQ ID NOs:1-18 or from an amino acid sequence that is at least 80% to 99% identical to the Fab binding domain selected from SEQ ID NOs:1-18.

In some aspects, the disclosure features an Fc varient library comprising the variants of Table 2 and/or variants comprising amino acid sequences that are at least 80% to 99% identical to the Fc variants of Table 2.

In some embodiments, the disclosure features the antibody or the composition disclosed herein, for use in treating an Ebola virus infection in a subject.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIG. 1 depicts a heat map of relative humoral responses induced in EVD survivors and household contacts. The Z-score for the response across a given feature was calculated and used to generate a one-way unsupervised hierarchical clustered heat map, in which patients (survivors or contacts) were clustered based on their overall humoral profile across the x-axis. Each feature measured is shown on the y-axis, and grouped by broad categories (effector function, antigen-specific antibody subclass/isotype levels, and antigen-specific antibody binding to Fc receptors). The response is color-coded by magnitude as indicated in the legend.

FIGS. 2A-2C depict diversity in antibody-mediated innate immune effector functions across human survivors of EVD.
A. Unsupervised hierarchical clustering of cluster EVD survivors (S-value); household contacts (C-value) and a USA-based healthy control (USA Neg.) based on induction of antibody-mediated innate immune effector functions. The heatmap shows the relative value of the indicated functions with purple and green representing the maximum and minimum values, respectively, across the samples as indicated in the legend.
B. Principal component analysis of 40 EVD survivors and K-means clustering to identify 10 distinct clusters.
C. Composite functional flower plots using the mean values for each cluster were overlaid onto the mirrored PCA. Each segment represents a different function, and the size of the segment represents the magnitude of the function relative to the entire panel as indicated in the inset legend. Cluster 9 has no detectable functional activity.

FIGS. 3A-3B depict protective efficacy and functional activity of internal fusion loop binding antibodies.
A. Survival curve of BALB/c mice infected with 1,000 PFU of maEBOV and treated at 2 days post-infection with 100 μg/mouse (10 mice/antibody) of the indicated antibodies. Data are adapted from (Saphire et al., 2018a) with permission.
B. Induction of the indicated innate immune effector functions (hu=human effector cell; m=mouse effector cell) and neutralization were measured. The composite effector profile for each antibody is shown, where the ability of the antibody to induce the indicated effector function above the negative control cutoff was considered positive for that function. The in vivo protective efficacy (% survival) is indicated for each antibody. Data are adapted from (Gunn et al., 2018; Saphire et al., 2018a) with permission.

FIGS. 4A-4C depict quality control of recombinant REFORMabs.
A. SDS-PAGE of a representative subset of REFORMabs (0.5 μg each) stained with Simply Blue stain.
B. Western blot analysis of a representative subset of REFORMabs. Membranes were probed with a FITC-conjugated anti-human IgG secondary antibody.
C. FPLC purification of representative antibodies with representative signatures for the antibodies produced at large-scale shown.

FIGS. 5A-5C depict functional characterization of the REFORMab panel.
A. VIC16 REFORMabs (5 μg/ml) were evaluated for induction of antibody-dependent monocyte phagocytosis (ADCP), neutrophil phagocytosis (ADNP), complement deposition (ADCD), NK cell degranulation (NK: CD107a), NK cell secretion of IFNγ(NK: IFNγ) and NK secretion of MIP-1β(NK: MIP-1β) following antibody incubation with recombinant Ebola glycoprotein (GP). The raw values for each effector function are plotted, and representative flow cytometry plots are shown in FIGS. 9A-D.
B. Plot of IC₅₀ (μg/ml)⁻¹ calculated from an 8-point, 2-fold dilution series of VIC16 REFORMabs in a neutralization assay with VSV pseudovirions expressing EBOV GP.
C. Functional profile of each REFORMab shown as a flower plot, in which each petal represents a different function, and the size of the petal represents the magnitude of the function relative to the entire panel as indicated in the legend.

FIGS. 6A-6E depict down-selection of REFORMabs for in vivo evaluation.
A. Unsupervised hierarchical clustering with two-way color coding was used to cluster antibodies according to induction of innate immune effector functions (top graph), and K-means clustering was used to group IgG1 VIC16 REFORMabs having a neutralization IC₅₀ of <1 μg/ml into 6 distinct clusters (bottom graph). Results for principal component analysis of mAbs within each cluster are shown, and the inset bi-ray plot indicates the loadings within the plot. The K-means clustering defined-cluster for each antibody is indicated below the heatmap.
B. The five down-selected REFORMabs were produced in larger quantities and re-evaluated in triplicate in dilution curves for neutralization of VSV-EBOV GP pseudovirions, and induction of antibody-dependent complement deposition (ADCD), neutrophil phagocytosis (ADNP), monocyte phagocytosis (ADCP), NK cell degranulation (NK: CD107a), NK cell secretion of IFN□ (NK: IFNγ), and NK secretion of MIP-1β (NK: MIP-1β). Antibodies were assayed for neutralization in a 3-fold dilution curve (3 μg/ml-0.0048 μg/ml); ADCD in a 2-fold dilution curve (10 μg/ml-0.16 μg/ml); and ADNP, ADCP, and ADNKA in a 5-fold dilution curve (5 μg/ml-0.00064 μg/ml).
C. The five down-selected REFORMabs were reproduced in larger quantities and re-evaluated for induction of effector function, and the AUC for each function across the down-selected in vivo panel is shown in a flower plot
D. Binding affinity for human FcγRs (FcγR3a F158, FcγR2A R131, FcγR2B, and FcγR3B) and mouse FcγRs (FcγRI FcγRIV, FcγR3, and FcγR2b) was measured for the selected antibodies by surface plasmon resonance (human FcγR) or biolayer inferometry (mouse FcγR) and K_{D} (M⁻¹) was plotted.
E. Binding of low-affinity murine FcγRs (mFcγRIIb and mFcγRIII) to mAb:Ebola GP immune complexes was determined using fluorescently labeled recombinant murine FcγRs at the indicated antibody concentrations. Median Fluorescent Intensity (MFI) of FcγR binding is plotted.

FIGS. 7A-7C depict the correlation between human and mouse FcγRs and effector functions. A subset of VIC16 REFORMabs were evaluated for binding to human FcγRs (3B, 3A F158, 2A H131, R131, and 2B) and mouse FcγRs (IV, III, and IIB) by SPR or Luminex (mFcγRI).
A. The K_{D} M⁻¹ or MFI (mFcγRI) was used to determine association by Spearman rho correlation between human box and mouse box FcγRs. The spearman r value is indicated for statistically significant associations after correction for multiple comparisons.
B. The K_{D} M⁻¹ or MFI (mFcγRI) was used to determine association by Spearman rho correlation between mouse and human FcγRs and effector functions, including human box, mouse box or neutralization box. The spearman r value is indicated for statistically significant associations after correction for multiple comparisons.
C. Correlation plots are shown for statistically significant associations for the indicated FcγRs, with outlier antibodies indicated.

FIGS. 8A-8D depict distinct functional profiles track with in vivo antibody-mediated protection.
A. Selected REFORMAbs were administered to mice (100 μg/mouse) 24 hours after infection with 1,000 pfu of mouse-adapted Ebola virus. Mice were monitored for 28 days post-infection, and survival of mice is plotted.
B. Clinical disease score (top row) and percentage of starting weight (bottom row) of mice is shown with different lines in each boxh representing individual mice. The percentage survival is indicated in the top right corner panels showing clinical disease score for each mAb.
C. Selected mAbs were administered to mice at a reduced dose (30 μg/mouse) 24 hours after infection with 1,000 pfu of mouse-adapted Ebola virus. Mice were monitored for 28 days post-infection, and the survival is plotted.
D. Clinical disease score (top row) and percentage of starting weight (bottom row) of mice treated with 30 μg of the indicated antibody is shown with different lines in each box representing individual mice.

FIGS. 9A-9D show representative flow cytometry plots.

Representative flow cytometry plots and gating strategy is shown for ADNP (A), ADCD (B), ADCP (C), and ADNKA (D).

FIG. 10 depicts CR3022 Fc-variant library. Each flower shows the functional potential of a given Fc-engineered CR3022 variant, where each leaf in a flower depicts a function (ADNP: antibody dependent neutrophil phagocytosis, ADCP: antibody dependent cellular phagocytosis, ADCD: antibody dependent complement deposition, ADNKA CD107a: antibody dependent NK cell degranulation (CD107a), cytokine secretion (IFNγ), chemokine secretion (MIP1b). The size of the leaf depicts the magnitude of the function driven by the specific Fc-variant.

FIG. 11 depicts protection in mice. The dot plot shows the viral loads in the lobes of the lung at day 3 following challenge. The line plot shows the trajectory of the weight across each animal group following infection. Each antibody is represented as follows: wildtype IgG1 (circle), Fc-enhanced (SDIE, square), Fc-knock out (KO, gray upright triangle), and a control antibody (black inverted triangle).

FIG. 12 depicts protection in hamsters. The dot plot shows the viral loads in the lungs of the hamsters 3 days following infection. The line plot shows the trajectory of the weight across each group. Each antibody is represented as follows: wildtype (circle), Fc-enhanced (SDIE, square), Fc-knockout (KO, gray upright triangle), and a control antibody (black inverted triangle).

FIGS. 13A-13B depict assembly schematic for full construct and light-chain construct. (a) A full construct is assembled using the BsaI overhangs shown, consisting of a custom vector (RL002), the variable heavy chain fragment (VH), the Fc variant (CH), a P2A/IL-2 signal sequence fragment (P2A/SS), a kappa or lambda variable light chain fragment (VL) and a kappa or lambda constant light chain fragment (CL). (b) A light chain construct consisting of a custom vector (RL003) and variable/constant light chain kappa/lambda fragments.

### DETAILED DESCRIPTION

### Definitions

Unless otherwise defined, all technical and scientific terms used herein have the same meaning as commonly understood by one of ordinary skill in the art to which this invention belongs. In case of conflict, the present application, including definitions will control.

A “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein “an increase” refers to an amount of functional activity that is at least about 0.05 fold (for example 0.1, 0.2, 0.3, 0.4, 0.5, 1, 5, 10, 25, 50, 100, 1000, 10,000-fold or more) greater than the amount of functional activity compared to a reference standard (e.g., a background level). “Increased” as it refers to an amount of functional acitvity also means at least about 5% (for example 5, 6, 7, 8, 9, 10, 15, 20, 25, 30, 35, 40, 45, 50, 55, 60, 65, 70, 75, 80, 85, 90, 95, 99 or 100%) greater than the amount of functional acitvity in a reference standard. Amounts can be measured according to methods known in the art for determining various metrics of functional activity.

As used herein “a decrease” refers to an amount of functional activity that is at least about 0.05 fold (for example 0.1, 0.2, 0.3, 0.4, 0.5, 1, 5, 10, 25, 50, 100, 1000, 10,000-fold or more) less than the amount of functional activity compared to a reference standard (e.g., a background level). “Decreased” as it refers to an amount of functional acitvity also means at least about 5% (for example 5, 6, 7, 8, 9, 10, 15, 20, 25, 30, 35, 40, 45, 50, 55, 60, 65, 70, 75, 80, 85, 90, 95, 99 or 100%) less than the amount of functional acitvity in a reference standard. Amounts can be measured according to methods known in the art for determining various metrics of functional activity.

Ranges provided herein are understood to be shorthand for all of the values within the range. For example, a range of 1 to 50 is understood to include any number, combination of numbers, or sub-range from the group consisting of 1, 2, 3, 4, 15 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein an “Fc domain” comprises constant domains from the antibody heavy chains. The Fc domain modulates immune cell activity through binding to effector cells and triggering various effects after the antibody Fab region binds to an antigen.

As used herein a “Fab binding domain” comprises a region on an antibody that binds to antigens. It is composed of one constant and one variable domain of each of the heavy and the light chain.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods of the Disclosure

Embodiments of the present disclosure are based, in part, on the development of an Fc-engineering system to rapidly graft distinct Fc-domains—each with a different Fc-functional property onto emerging disease-specific monoclonal antibodies. The present disclosure provides a method for producing a monoclonal antibody with a profile of functional activity directed against an antigen of interest (“a functional profile”); said method comprising the steps of:
a) generating a library of IgG1 Fc domains, each comprising a different Fc mutation, thereby generating Fc variants; and
b) generating plasmids encoding each of the Fc variants linked to an Fab binding domain, wherein the Fab binding domain comprises variable heavy and light chains of an antibody that is reactive against the antigen of interest, thereby forming a Fab-Fc variant; and
c) expressing the Fab-Fc variants from the plasmids; and
d) determining the functional profile of each Fab-Fc variant, thereby producing a monoclonal antibody with a profile of functional activity directed against the antigen of interest.

The antigen of interest can be present, for example, on a pathogen or a cell undergoing unwanted proliferation (e.g., tumor cell or cancer cell).

Antibodies are glycoproteins that bind specific antigens. They are produced in response to invasion by foreign molecules in the body. Antibodies exist as one or more copies of a Y-shaped unit, composed of four polypeptide chains. Each Y contains two identical copies of a heavy chain, and two identical copies of a light chain, which are different in their sequence and length. The top of the Y shape contains the variable region, which binds tightly and specifically to an epitope on the antigen.

The light chains of an antibody can be classified as either kappa or lambda type, based on small differences in polypeptide sequence. The heavy chain makeup determines the overall class of each antibody. In mammals, antibodies are divided into five isotypes: IgG, IgM, IgA, IgD and IgE, based on the number of Y units and the type of heavy chain. The isotypes differ in their biological properties, functional locations and ability to deal with different antigens. The type of heavy chain present defines the class of an antibody. There are five types of mammalian Ig heavy chain denoted by Greek letters: alpha, delta, epsilon, gamma and mu. These chains are found in IgA, IgD, IgE, IgG and IgM antibodies, respectively. Heavy chains differ in size and composition; alpha and gamma contain approximately 450 amino acids, while mu and epsilon have approximately 550 amino acids.

Each heavy chain has two regions, the constant region and the variable region. The constant region is identical in all antibodies of the same isotype, but differs in antibodies of different isotypes. Heavy chains gamma, alpha and delta have a constant region composed of three tandem Ig domains and a hinge region for added flexibility. Heavy chains mu and epsilon have a constant region composed of four immunoglobulin domains. The variable region of the heavy chain differs depending on the B cell that produced it but is the same for all antibodies produced by a single B cell or B cell clone. The variable region of each heavy chain is approximately 110 amino acids long and is composed of a single Ig domain.

In mammals, there are only two types of light chain, lamda and kappa. A light chain has two successive domains: one constant domain and one variable domain. The approximate length of a light chain is 211-217 amino acids. Each antibody contains two light chains that are always identical.

The Y-shape of an antibody(e.g., immunoglobin G) can be divided into three sections: two F(ab) regions and an Fc region. The F(ab) regions contain the variable domain that binds to cognate antigens. The Fc fragment provides a binding site for endogenous Fc receptors on the surface of lymphocytes, and is also the site of binding for secondary antibodies. In addition, dye and enzymes can be covalently linked to antibodies on the Fc portion of the antibody for experimental visualization. These three regions can be cleaved into two F(ab) and one Fc fragments by the proteolytic enzyme pepsin.

Embodiments of the present disclosure are additionally based, in part, on the synthesized monoclonal antibodies, which have a profile of functional activity directed against an antigen of interest (“a functional profile”), wherein the antibodies are Fab-Fc variants comprising an IgG1 Fc domain having an amino acid sequence comprising at least one mutation relative to the native IgG1 Fc domain and an Fab binding domain, wherein the Fab binding domain comprises variable heavy and light chains of an antibody that is reactive against the antigen of interest, and wherein the functional profile comprises antibody-dependent complement deposition (ADCD), antibody dependent monocyte phagocytosis (ADCP), antibody-dependent neutrophil phagocytosis (ADNP), antibody-dependent NK cell activation (ADNKA) as measured by the activation markers CD107a, MIP1b and IFNγ and binding affinity to FcyRs. Embodiments of the present disclosure include Fab-Fc variants having at least 80% to 99% (e.g., 80%, 85%, 90%, 95% or 99%) amino acid sequence identity to an Fab-Fc variant identified according to the methods of the disclosure. In specific embodiments, the Fab-Fc variants are selected from Table 6 or from amino acid sequences that are at least 80% to 99% (e.g., 80%, 85%, 90%, 95% or 99%) identical to the Fab-Fc variants of Table 6.

In various embodiments of the disclosure, libraries of IgG1 Fc domains are generated, each comprising a different Fc mutation, thereby generating Fc variants. Fc Mutations are introduced by site-directed mutagenesis (SDM) using methods known in the art, for example, using DNA primers that introduce single-site mutations to a polymerase chain reaction (commercial kits for SDM include the Q5® Site-Directed Mutagenesis Kit by New England BioLabs). These methods are known in the art, and one of skill will be able to identify such methods as appropriate in light of the present disclosure. Exemplary Fc variant libraries of the disclosure are provided in Table 2. Embodiments of the present disclosure include Fc variants having at least 80% to 99% (e.g., 80%, 85%, 90%, 95% or 99%) amino acid sequence identity to Fc variants identified and/or constructed according to the methods of the disclosure. In specific embodiments, the Fc variants are selected from Table 2 or from amino acid sequences that are at least 80% to 99% (e.g., 80%, 85%, 90%, 95% or 99%) identical to the Fc variants of Table 2.

In various embodiments of the disclosure, plasmids are generated encoding each of the Fc variants linked to an Fab binding domain, wherein the Fab binding domain comprises variable heavy and light chains of an antibody that is reactive against an antigen of interest, thereby forming a Fab-Fc variant; and the Fab-Fc variants are expressed from the plasmids. Plasmids are designed to be compatible with the Type II restriction digest used to assemble the complete antibody variant(s). Exemplary Fab binding domains of the disclosure are provided in SEQ ID NOs:1-18. Embodiments of the present disclosure include Fab binding domains having at least 80% to 99% (e.g., 80%, 85%, 90%, 95% or 99%) amino acid sequence identity to Fab binding domains identified and/or constructed according to the methods of the disclosure. In specific embodiments, the Fab binding domains are selected from SEQ ID NOs:1-18 or from an amino acid sequence that is at least 80% to 99% (e.g., 80%, 85%, 90%, 95% or 99%) identical to the Fab binding domains of SEQ ID NOs:1-18.

In certain embodiments, the expression vector comprises a regulatory sequence or promoter operably linked to the nucleotide sequence encoding the Fab-Fc variants. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Nucleic acid sequences encoding Fab-Fc variants preferably have strong promoters that are active in a variety of cell types. The promoters for eukaryotic nucleic acid sequences are typically present within the structural sequences encoding the Fab-Fc variants. Although there are elements which regulate transcriptional activity within the 5′ upstream region, the length of an active transcriptional unit may be considerably less than 500 base pairs.

Additional exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein.

In various embodiments of the disclosure, the functional profile of each Fab-Fc variant is determined to identify variants having effective activity against an antigen and/or pathogen of interest. Determining the functional profile comprises determining the level of at least one of phagocytosis of monocytes and/or neutrophils; complement deposition; NK cell degranulation; NK cell secretion of cytokine IFNγ and chemokine MIP-1β and expression of membrane protein CD107a; neutralizing activity; and FcyR binding.

Cytokine IFNγ (Interferon-gamma), is known in the art as a cytokine critical for innate and adaptive immunity against viral and intracellular bacterial infections and for tumor control.

Chemokine MIP-1β is an 8-kD acidic protein that is knownin the art to be upregulated upon stimulation in monocytes, T cells, and other lymphocytes. It belongs to the CC chemokine subfamily and directs the migration of specific subsets of leukocytes.

Membrane protein CD107a is a 120-kD lysosomal membrane glycoprotein that is an acidic, heavily glycosylated membrane protein enriched in the lysosomal membrane. It is a marker of CD8+ T-cell degranulation following stimulation.

In various embodiments of the disclosure, the Fab-Fc is preceeded with a secretion signal, for example the IL-2 singal sequence that directs the antibody protein to be secreted (Zhang et al., 2005,).

Antibody-dependent NK cell activation (ADNKA) can be determined by exemplary methods described herein below. ADNKA can be quantified by measuring the percentage of NK cells producing CD107a, MIP1β and IFNγ through permeabilized antibody staining and flow cytometry. For example, antigen is coated onto MaxiSorp 96-well plates at 300 ng/well and incubated at 4° C. overnight before being washed with PBS and blocked with 5% BSA. Antibody is added to a final concentration of 2-20 ug/mL and incubated for 2 hours at 37° C. for adsorption. A background control sample is prepared by replacing the antibody with PBS. Unbound antibodies are removed by washing with PBS and 50,000 NK cells, enriched from Ficol separation of human blood donor samples, are added per well in the presence of 4 ug/mL brefeldin A, 5 ug/mL GolgiStop and CD107a antibody (clone H4A3, PE-Cy5 labeled) in cell culture media (RPMI+10% FBS+1% Pen/Strep). The wells are incubated for 5 hours at 37° C. NK cells are stained with CD56 antibody (clone B159, PE-Cy7 labeled) and CD16 antibody (clone 3G8, APC-Cy7 labeled) and CD3 antibody (clone SP34-2, Pacific Blue labeled). Cells are then permeabilized and stained with IFNγ antibody (clone B27, FITC labeled) and MIP-1b antibody (clone D21-1351, PE labeled) and analyzed by flow cytometry. NK cells are identified as SSC-A^{high}, CD66b+, CD3- and CD14-using a fluorescence-minus-one gating strategy common in the field and quantified as the percentage of CD107a+, MIP1b+ and/or IFNγ+cells. Statistically significant activation is defined independently for each marker (CD107a+, MIP1b+, or IFNγ) as a percentage that is three standard deviations above the percentage of the background sample based on replicate samples.

Antibody-dependent neutrophil phagocytosis (ADNP) can be determined by exemplary methods described herein below. ADNP is quantified by measuring phagocytic uptake of FITC fluorescent beads. Yellow-green/FITC Neutravidin beads (Life Technologies) are coupled to antigen and incubated with antibody at a final concentration of 2-20 ug/mL for 2 hours at 37° C. in culture medium (RPMI+10% FBS+1% Pen/Strep). A “background” sample is prepared in the same manner except antibody is substituted with PBS. Freshly isolated white blood cells from human donor peripheral blood are added at 50,000 per antibody-antigen sample and incubated for 1 hour at 37° C. Neutrophils are stained with fluorescently labeled CD66b antibody (clone G10F5, labeled with Pacific Blue), fixed for 20 minutes at room temperature in 4% paraformaldehyde and a “cell background” sample is prepared with cells only. The cells are then anlyzed by flow cytometry. Neutrophils are defined as SSC-A^{high}, CD66b+ using a fluorescence-minus-one gating strategy. Bead-positive cells are defined and gated by comparison to the “cell background” cells. A phagocytic score is calculated for the “background” sample, reflecting antibody-independent phagocytic activity, as the percentage of bead-positive cells multiplied by the mean fluorescence of the bead-positive population. Phagocytic scores are calculated for experimental samples and a significant level of phagocytosis is defined as a phagocytic score that is three standard deviations above the background phagocytic score based on replicate samples.

Antibody-dependent cellular phagocytosis (ADCP) can be determined by exemplary methods described herein below. ADCP is measured in the same way as ADNP, with the following exceptions: THP-1 cells are used at a density of 25,000 per sample and cell identity marker antibodies are not used (e.g. CD66b). A significant level of phagocytosis is defined as a phagocytic score that is three standard deviations above the background phagocytic score based on replicate samples.

Antibody-dependent complement deposition (ADCD) can be determined by exemplary methods described herein below. ADCD is measured through complement (C3) deposition onto antigen-coated, antibody-bound beads. Antigen-coated beads are prepared and coated with antibody in the same way as described for ADNP. A “background” sample is prepared with no antibody. Guinea pig complement (Cedarlane Labs) is reconstituted in water according to the manufacturers instructions and diluted approximately 1:50 in veronal buffer with calcium and magnesium (Boston Bioproducts). 150 uL diluted complement is added to 20 uL of antibody/bead mixture and incubated for 20 mintutes at 37° C. before being washed with 15 mM EDTA in PBS and stained with FITC-conjugated goat IgG fraction to guinea pig C3 (MP Biomedicals). C3 deposition is analyzed by flow cytometry. The mean FITC fluorescence is calculated for the “background” and experimental samples. Complement deposition is defined as any mean fluorescence that is three standard deviations above background based on replicate samples.

Fc receptor (FcR) binding can be determined by exemplary methods described herein below. (FcR) binding can be measured in a multiplex bead binding assay. Magplex Luminex beads are coupled to antigen by activating the beads for 30 minues at room temperature in 100 mM monobasic sodium phosphate, 5 mg/mL sulfo-NHS and 5 mg/mL ethyl dimethylaminopropyl carbodiimide hydrochloride at pH 6.2. Beads are washed with 50 mM MES at pH 5.0 and incubated with antigen (10-75 ug) for 2 hours on a rotator. Anigen-coupled beads are blocked with 0.1% BSA, 0.02% Tween-20 and 0.05% Azide, pH 7.4. Coupled beads are washed in PBS-Tween, resuspended in PBS and stored at 4° C. Purified Fc receptor protein is prepared by biotinylating using a BirA500 kit (Avidity, manufacturers instructions). Beads are diluted to a concentration of 100 microspheres per antigen/μL in 0.1% BSA in PBS. Antibodies are diluted to 5 ug/mL in PBS with 0.1% BSA and then diluted 1:10 with diluted bead mixture in a black, clear-bottom, 384-well plate; and incubated at 4° C. for 16 hours with shaking at 900 rpm. A “background” sample is prepared in the same way except without antibody. After the incubation, plates are washed with 0.1% BSA in PBS. Biotinylated FcRs are labeled with streptavidin-PE (Prozyme, PJ31S) for 10 minutes. PE-labeled FcRs are added to plates and incubated for 1 hour at room temperature on a shaker. Plates are washed with 0.1% BSA in PBS and resuspended in Qsol Buffer (Intellicyt). PE fluorescence analyzed by flow cytometry. FcyR binding is defined as a mean fluorescence that is three standard deviations above background based on replicate samples.

Neutralization can be measured using either pseudotyped virus or live virus and appropriate host/target cell lines that have or have not been transformed with an infection reporter gene such as luciferase, gaussian luciferase or GFP (for example). The specific method used depends on whether the virus may be pseudotyped, the reporter used in the pseudotype and the reporter cell line used. Antibody dilutions are prepared in a culture media appropriate for the target cell ine. Virus is added and incubated for 30-60 minutes at 37° C. An infection control is prepared with virus and no antibody and a background control is prepared with no virus or antibody. A 96-well plate with a confluent monolayer of target cells is inoculated and incubated at 37° C. for 24-72 hours depending on the pseudotype/virus and cell line used. Infection is quantified in a manner appropriate for the reporter (e.g. GFP is measured by flow cytometry, luciferase is measured by lysing the cells and using a luminescence plate reader). Normalized infection is determined from flow cytometry reporters (e.g. GFP) by gating out cells from the background sample to obtain a percentage of reporter+cells, which is normalized to the percent of reporter+cells in the virus control sample. Normalized infection is obtained from luminescence reporters (e.g. luciferase) by subtracting the luminescence of the background control and normalizing to the luminescence of the virus control. Percent neutralization is calculated as 1-% infection. The IC50 (inhibitory concentration at 50%) is calculated as the concentration giving 50% neutralization through either interpolation or fitting to the Hill/Median Effect models. An antibody is considered neutralizing if it has an IC50 at or below 50 μg/mL.

Pathogens of interest targeted by the Fab-Fc variants of the disclosure include the Ebola virus. The Fab binding domain of the synthesized Fab-Fc variants of the disclosure comprises variable heavy and light chains of antibodies known in the art to be reactive against the pathogen of interest.

### Vic16 Antibodies

In specific embodiments, the pathogen of interest is an Ebola virus and the antibody that is reactive against the Ebola virus and from which the Fab domain is derived is VIC16. In some embodiments, the Vic16 antibody binds to the Ebola virus glycoprotein. The VIC16 antibody is described in Saphire E O, et al. Cell. 2018 Aug. 9; 174(4):938-952.e13.

The amino acid sequences of the complementarity determining regions (CDRs) and the mature heavy chain variable regions and light chain variable regions of the VIC16 antibody are shown below. The CDRs described herein include the Kabat CDR definitions Kabat, E. A., Wu, T. T., Perry, H. M., Gottesman, K. S. & Foeller, C. (1991). Heavy chain CDR1: GYTFTKYW (SEQ ID NO: 19); Heavy chain CDR2: INPSTGYS (SEQ ID NO: 20); Heavy chain CDR3: VRGYDSHYYVMDY (SEQ ID NO: 21); Light chain CDR1: ESVEYYGTTL (SEQ ID NO: 22); Light chain CDR2: GAS; Light chain CDR3: QQSRKVPYT (SEQ ID NO: 24). The CDRs are shown as the bold positions in the Vic16 variable heavy (VH) and variable light (VL) domain amino acid sequences below:

Vic16 VH domain:
(SEQ ID NO: 25)
QVQFQQSGAELAKLGASVKMSCKASGYTFTKYWMHWIKQRPGQGLEWIG
YINPSTGYSENNQKFKGKAILTADKSSSTAYMQLSSLTSDDSAVYYCVR
GYDSHYYVMDYWGQGTSVTVSS
Vic16 VL domain:
(SEQ ID NO: 26)
DIVITQDTASLAVSRGQRATISCRASESVEYYGTTLMQWYQQRPGQPPK
LLIYGASNVESGVPARFSGSGSGTDFSLNIHPVEEDDIAMYFCQQSRKV
PYTFGGGTKLEIK

In some embodiments, the Vic16 antibody constant heavy and light chain amino acid sequences are in Table 1 as follows for an exemplary Vic16 antibody (Vic16-KWES).

TABLE 1
Fc-enhanced Vic16 antibody
constant heavy and light chain

| | SEQ | |
|---|---|---|
| Description | ID NO: | Sequence |
| Vic16-KWES | SEQ ID | ASTKGPSVFPLAPSSKSTSGGTAALGC |
| constant heavy | NO: 27 | LVKDYFPEPVTVSWNSGALTSGVHTFP |
| (CH)1 domain | | AVLQSSGLYSLSSVVTVPSSSLGTQTY |
| | | ICNVNHKPSNTKVDKKV |
| Vic16-KWES | SEQ ID | PCPAPELLGGPSVFLFPPKPKDTLMIS |
| CH2 domain | NO: 28 | RTPEVTCVVVDVSHEDPEVKFNWYVDG |
| | | VEVHNAKTKPREEQYNSTYRVVSVLTV |
| | | LHQDWLNGKEYKCKVSNWALPAPISKT |
| | | ISKAK |
| Vic16-KWES | SEQ ID | GQPREPQVYTLPPSREEMTKNQVSLTC |
| CH3 domain | NO: 29 | LVKGFYPSDIAVEWESNGQPENNYKTT |
| | | PPVLDSDGSFFLYSKLTVDKSRWQQGN |
| | | VFSCSVMHEALHNHYTQKSLSLSPGK |
| Vic16 constant | SEQ ID | RTVAAPSVFIFPPSDEQLKSGTASVVC |
| light (CL) | NO: 31 | LLNNFYPREAKVQWKVDNALQSGNSQE |
| domain | | SVTEQDSKDSTYSLSSTLTLSKADYEK |
| | | HKVYACEVTHQGLSSPVTKSFNRGEC |

*The linker sequence ASTKG (SEQ ID NO: 32) is bolded
in SEQ ID NO: 27. The Vic16 antibody hinge region
between CH1 and CH2: EPKSCDKTHTCP (SEQ ID NO: 30).

In some embodiments, the VIC16 antibody of the disclosure has an Fab region and an Fc region. The Fab region is comprised of the VH and VL domains (SEQ ID NOs: 25 and 26). In some embodiments, the VIC16 antibody of the disclosure has an Fc region thatcomprises the amino acid sequence set forth in SEQ ID NO: 53. In some embodiments, the VIC16 antibody of the disclosure has an Fc region that comprises the amino acid sequence set forth in SEQ ID NO: 70. In some embodiments, the VIC16 antibody of the disclosure has an Fc region that comprises the amino acid sequence set forth in SEQ ID NO: 74.

In some embodiments, the VIC16 antibody of the disclosure has an Fc region comprising an amino acid sequence that is at least 80%, 85%, 90%, 95%, 97%, 98% or 99% identical to the amino acid sequence of any one of SEQ ID NOs: 97-104 and 106- 159.

In some embodiments, the VIC16 antibody of the disclosure has the amino acid sequence set forth in SEQ ID NO: 99. In some embodiments, the VIC16 antibody of the disclosure has the amino acid sequence set forth in SEQ ID NO: 131. In some embodiments, the VIC16 antibody of the disclosure has the amino acid sequence set forth in SEQ ID NO: 139.

### Uses and Methods of the Fc-enhanced VIC16 Antibodies of the Disclosure

The VIC16 antibodies of the disclosure can be prepared, characterized and tested using methods well-known in the art and described, for example, in U.S. Patent Application No. 2021/0095007, and U.S. Pat. No. 10,640,550, both of which are disclosed herein in their entireties.

In some embodiments, the antibodies and compositions of the disclosure can be used to treat and/or prevent (e.g. , immunize against) Ebola virus infection in a subject . In other aspects, the antibodies, and compositions of the disclosure can be used to detect Ebola virus infection in a sample. In some embodiments, the disclosure provides use of VIC16 antibodies described herein in the manufacture or preparation of a medicament for the treatment of an Ebola virus infection. In some embodiments, a VIC16 antibody described herein is for use in the treatment of an Ebola virus infection.

Ebola virus disease (EVD) caused by infection with viruses of the genus Ebolavirus, i.e., ebolaviruses has an average fatality rate of about 50%, and the fatality rate in the past epidemic is in the range of 25% to 90%. The incubation period from infection with ebolaviruses to development of EVD is 2 to 21 days. Early symptoms of EVD are fatigue fever, myalgia, headache and sore throat, which are followed by vomiting, diarrhea, exanthema, renal and hepatic dysfunction, external hemorrhage, and other symptoms.

For use in therapy, the antibodies of the disclosure can be administered to a subject directly (i.e., in vivo) , either alone or with other therapies such as an immunostimulatory agent or an antibody cocktail. In all cases, the antibodies, compositions, immunostimulatory agents and/or other therapies are administered in an effective amount to exert their desired therapeutic effect.

In some embodiments, the antibodies of the disclosure can be combined with monoclonal antibodies against Ebola virus glycoprotein, such as characterized in U.S. Pat. No. 6,630,144, Olinger et al., PNAS 2012; 109, 18030-18035, and Pettitt et al., Sci Transl Med 2013; 5, 199ra113. In some embodiments, the antibodies of the disclosure can be combined with other therapeutic antibodies such as Inmazeb™ (a cocktail of atoltivimab, maftivimab, odesivimab-ebgn), and Ebanga™ (ansuvimab-zykl).

Preferred routes of administration include, for example, injection (e.g., subcutaneous, intravenous, parenteral, intraperitoneal, intrathecal, etc). The injection can be in a bolus or a continuous infusion. Other routes of administration include oral administration. Antibodies of the disclosure also can be coadministered with adjuvants and other therapeutic agents. It will be appreciated that the term “coadministered ” as used herein includes any or all of simultaneous, separate, or sequential administration of the antibodies and conjugates of the present disclosure with adjuvants and other agents, including administration as part of a dosing regimen.

The antibodies are typically formulated in a carrier alone or in combination with such agents. Examples of such carriers include solutions, solvents, dispersion media, delay agents, emulsions and the like. The use of such media for pharmaceutically active substances is well known in the art. Any other conventional carrier suitable for use with the molecules falls within the scope of the disclosure.

In certain embodiments, the antibody is administered to a subject for treatment of an Ebola virus infection. The subject can be administered a composition comprising the antibodies disclosed herein alone or in combination, along with a therapeutic agent. In certain embodiments, the therapeutic agent is interferon alpha, one or more of therapeutic antibodies, including but not limited to Inmazeb™ (a cocktail of atoltivimab, maftivimab, odesivimab-ebgn), and Ebanga™ (ansuvimab-zykl). The combinations described herein may be more effective at neutralizing Ebola virus. On some embodiments, treating a subject with an Ebola infection enhaces the subject.

In various embodiments, the compositions comprising synthesized antibodies can be fomulated to contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It should also be suitable for use in contact with any tissues or organs with which it may come in contact, meaning that it should not carry a risk of toxicity, irritation, allergic response, immunogenicity, or any other complication that excessively outweighs its therapeutic benefits.

In various embodiments, the compositions comprising synthesized antibodies include a pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients that are pharmaceutically acceptable and compatible with the active ingredient and in amounts suitable for use in the therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic composition as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent used in the disclosure that will be effective in the treatment of a particular disorder or condition will depend on the nature of the disorder or condition, and can be determined by one of skill in the art with standard clinical techniques.

The synthesized antibodies described herein can be delivered in a therapeutically effective amount. The precise therapeutically effective amount is that amount of the synthesized antibodies that will yield the most effective results in terms of efficacy of treatment in a given subject. The therapeutically effective amount of synthesized antibody will induce an immune response in the subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

For the treatment of the disease, the appropriate effective amount of the synthesized antibodies described herein depends on the type of disease to be treated, the severity and course of the disease, the responsiveness of the disease, and the desired clinical outcome, previous therapy, and patient's clinical history. The dosage can also be adjusted by one of skill in the art in the event of any complication and at the discretion of a person skilled in the art. The person skilled in the art can determine optimum dosages, dosing methodologies and repetition rates. The synthesized antibodies can be administered one time or over a series of treatments lasting from several days to several months, or until a cure is effected or a diminution of the disease state is achieved (e.g., treatment or amelioration of the disease). The duration of treatment depends upon the subject's clinical progress and responsiveness to therapy. In certain embodiments, dosage is from 0.01 .mu.g to 100 mg per kg of body weight, and can be given once or more daily, weekly, monthly or yearly. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In various other embodiments, the synthesized antibodies described herein are administered in a series of treatments. In selected embodiments, the synthesized engineered antibodies described herein are administered to patients who have previously undergone a treatment. In some embodiments, the treatment is administered in any order, including prior to, concurrently with, substantially simultaneously or subsequent the administration of a second treatment.

The present disclosure is additionally described by way of the following illustrative, non-limiting Examples that provide a better understanding of the present disclosure and of its many advantages.

### EXAMPLES

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

### Materials and Methods for Examples 1-4.

Patient samples: Plasma samples were collected from individuals with documented clinical history of Ebola virus disease (EVD) approximately 3-4 years after infection and from household contacts of EVD survivors. At the time of acute disease, EVD patients received standard of care at Kenema Government Hospital (e.g., intravenous fluid replacement and administration of antibiotics as determined by the treating physician). Peripheral blood mononuclear cells were collected from MGH blood bank donations for use in innate immune effector assays. All subjects provided written consent. This study was approved by the Institutional Review Board of the Human Subjects Committee of MGH, Tulane University Institutional Review Board, and the Sierra Leone Ethics and Scientific Review Committee.

Plasmid design and construction: Five donor pUC plasmids encoding the variable heavy domain flanked by 5′ leader (GTCCTAGCTCTTCACGGTCTCCCAGC; SEQ ID NO: 161) and 3′ tail (GCCATGAGACCTTCGAAGAGCGTCGTA; SEQ ID NO: 162), the Fc domain flanked by 5′ leader (GTCCTGGCTCTTCACGGTCTCC; SEQ ID NO: 163) and 3′ tail (CGGATGAGACCTTCGAAGAGCGACGTC; SEQ ID NO: 164), a furin P2A and IL-2 signal sequence, the variable light domain flanked by 5′ leader (GCTCTAGCTCTTCACGGTCTCCCTCC; SEQ ID NO: 165) and 3′ tail (AAGATGAGACCTTCGAAGAGCTAGGCA; SEQ ID NO: 166) for kappa isoltype or flanked by 5′ leader (GCTCTAGCTCTTCACGGTCTCCCTCC; SEQ ID NO: 167) and 3′ tail (GGTCTGAGACCTTCGAAGAGCTCGCTG; SEQ ID NO: 168) for lambda isotype, and the constant light chain (kappa or lambda as appropriate for the variable light domain) flanked by 5′ leader (GCTCTAGCTCTTCACGGTCTCC; SEQ ID NO: 169) and 3′ tail (TCAGTGAGACCTTCGAAGAGCTAGGCA; SEQ ID NO: 170) were designed (FIGS. 13A-13D). Two destination vectors were designed that contained an IL-2 secretion signal, the suicide gene ccdB flanked by BsaI sites (RL002 and RL003). A library of 63 Fc variants were synthesized and cloned into the pUC donor plasmids for the Fc domain (Table 2).

TABLE 2
List of REFORM variants generated for this study. Specific amino acid
mutations are shown, and the antibody-dependent cellular cytotoxicity
(ADCC), antibody-dependent cellular phagocytosis (ADCP), antibody-dependent
complement deposition (ADCD) or binding to FcRn is indicated. Studies
describing the effect of the indicated mutation are listed.

| Fc Variant | AA Mutations | Description | Ref. |
|---|---|---|---|
| IgG1 | | Human IgG1 | |
| LALA | L234A/L235A | no FcγR | Jefferis R, et al. |
| | | binding | Mol Immunol. |
| | | | 1990 December; |
| | | | 27(12): 1237-40 |
| LALALS | L234A/L235A/M428L/N434S | no FcγR | (Zalevsky et al., |
| | | binding ↑FcRn | 2010) |
| LALAPG | L234A/L235A/P329G | no FcγR | Lo M, et al. J |
| | | binding | Biol Chem. |
| | | | 2017 Mar. 3; |
| | | | 292(9): 3900-3908 |
| N297Q | N297Q | Aglycosylated | Arnold JN, et al. |
| | | | Annu Rev |
| | | | Immunol. |
| | | | 2007; 25: 21-50 |
| N297QLS | N297Q/M428L/N434S | Aglycosylated; | (Zalevsky et al., |
| | | ↑FcRn | 2010) |
| SAEAKA | S298A/E333A/K334A | ↑ ADCC | (Lazar et al., |
| | | | 2006; Shields et |
| | | | al., 2001) |
| SAEAKALS | S298A/E333A/K334A/M428L/ | ↑ ADCC | (Lazar et al., |
| | N434S | ↑FcRn | 2006; Shields et |
| | | | al., 2001; |
| | | | Zalevsky et al., |
| | | | 2010) |
| LPLIL | F243L/R292P/Y300L/V305I/ | ↑ ADCC | (Stavenhagen et |
| | P396L | | al., 2007) |
| LPLILLS | F243L/R292P/Y300L/V305I/ | ↑ ADCC ↑ | (Stavenhagen et |
| | P396L; M428L/N434S | FcRn | al., 2007; |
| | | | Zalevsky et al., |
| | | | 2010) |
| E345R | E345R | ↑ ADCD | (Diebolder et |
| | | | al., 2014) |
| E345RLS | E345R/M428L/N434S | ↑ ADCD ↑ | (Diebolder et |
| | | FcRn | al., 2014; |
| | | | Zalevsky et al., |
| | | | 2010) |
| HFST | H268F/S324T | ↑ ADCD | (Moore et al., |
| | | | 2010) |
| HFSTLS | H268F/S324T/M428L/N434S | ↑ ADCD | (Moore et al., |
| | | ↑FcRN | 2010; Zalevsky |
| | | | et al., 2010) |
| S324T | S324T | ↑ ADCD | (Moore et al., |
| | | | 2010) |
| S324TLS | S324T/M428L/N434S | ↑ ADCD ↑ | (Moore et al., |
| | | FcRn | 2010; Zalevsky |
| | | | et al., 2010) |
| RGY | E345R/E430G/S440Y | ↑ ADCD | (Diebolder et |
| | | | al., 2014) |
| RGYLS | E345R/E430G/S440Y/M428L/ | ↑ ADCD ↑ | (Diebolder et |
| | N434S | FcRn | al., 2014; |
| | | | Zalevsky et al., |
| | | | 2010) |
| SEHFST | S267E/H268F/S324T | ↑ ADCD | (Moore et al., |
| | | | 2010) |
| SEHFSTLS | S267E/H268F/S324T/M428L/ | ↑ ADCD ↑ | (Moore et al., |
| | N434S | FcRn | 2010; Zalevsky |
| | | | et al., 2010) |
| KWES | K236W/E333S | ↑ ADCD | (Steurer et al., |
| | | | 1995) |
| KWESLS | K236W/E333S/M428L/N434S | ↑ ADCD ↑ | (Steurer et al., |
| | | FcRn | 1995; Zalevsky |
| | | | et al., 2010) |
| E333A | E333A | ↑ ADCD, | (Idusogie et al., |
| | | ↑ADCC | 2000; Idusogie |
| | | | et al., 2001) |
| I332E | I332E | ↑ ADCC | (Lazar et al., |
| | | ↑ADCP | 2006) |
| I332ELS | I332E/M428L/N434S | ↑ ADCC | (Lazar et al., |
| | | ↑ADCP ↑ | 2006; Zalevsky |
| | | FcRn | et al., 2010) |
| IEGA | G236A/I332E | ↑ ADCC | (Richards et al., |
| | | ↑ADCP | 2008) |
| IEGALS | G236A/I332E/M428L/N434S | ↑ ADCC, | (Richards et al., |
| | | ↑ADCP, ↑ | 2008; Zalevsky |
| | | FcRn | et al., 2010) |
| SDIE | S239D/I332E | ↑ ADCC | (Lazar et al., |
| | | ↑ADCP | 2006; Richards |
| | | | et al., 2008) |
| SDIELS | S239D/I332E/M428L/N434S | ↑ ADCC, | (Lazar et al., |
| | | ↑ADCP ↑ | 2006; Richards |
| | | FcRn | et al., 2008; |
| | | | Zalevsky et al., |
| | | | 2010) |
| SDIEAL | S239D/A330L/I332E | ↑ ADCC, | (Lazar et al., |
| | | ↑ADCP | 2006; Moldt et |
| | | | al., 2011) |
| SDIEALLS | S239D/A330L/I332E/M428L/ | ↑ ADCC, | (Lazar et al., |
| | N434S | ↑ADCP, ↑ | 2006; Moldt et |
| | | FcRn | al., 2011; |
| | | | Zalevsky et al., |
| | | | 2010) |
| SDIEALYTE | S239D/A330L/I332E/M252Y/ | ↑ ADCC, | (Dall′Acqua et |
| | S254T/T256E | ↑ADCP | al., 2002; Lazar |
| | | | et al., 2006) |
| SDIEALYTELS | S239D/A330L/I332E/M252Y/ | ↑ADCP, ↑ | (Dall′Acqua et |
| | S254T/T256E/M428L/N434S | FcRn | al., 2002; |
| | | | Zalevsky et al., |
| | | | 2010) |
| SDIEGA | G236A/S239D/I332E | ↑ ADCC, | (Moldt et al., |
| | | ↑ADCP | 2011; Richards |
| | | | et al., 2008) |
| SDIEGALS | G236A/S239D/I332E/M428L/ | ↑ ADCC, | (Moldt et al., |
| | N434S | ↑ADCP, ↑ | 2011; Richards |
| | | FcRn | et al., 2008; |
| | | | Zalevsky et al., |
| | | | 2010) |
| SDIESA | S239D/S298A/I332E | ↑ ADCC, | Shields RL, et |
| | | ↑ADCP | al., J Biol |
| | | | Chem. 2001 |
| | | | Mar. 2; |
| | | | 276(9): 6591-604. |
| | | | Lazar GA et al. |
| | | | Proc Natl Acad |
| | | | Sci USA. 2006 |
| | | | Mar. 14; |
| | | | 103(11): 4005-10 |
| SDIESALS | S239D/S298A/I332E/M428L/ | ↑ ADCC, | (Zalevsky et al., |
| | N434S | ↑ADCP, ↑ | 2010) |
| | | FcRn | |
| SDIEALGA | G236A/S239D/A330L/I332E | ↑ ADCC, | (Smith et al., |
| | | ↑ADCP | 2012) |
| SDIEALGALS | G236A/S239D/A330L/I332E/ | ↑ ADCC, | (Smith et al., |
| | M428L/N434S | ↑ADCP, ↑ | 2012; Zalevsky |
| | | FcRn | et al., 2010) |
| K326W | K236W | ↑ ADCD, | (Idusogie et al., |
| | | ↑ADCC | 2000; Idusogie |
| | | | et al., 2001) |
| K326WLS | K236W/M428L/N434S | ↑ ADCD, | (Idusogie et al., |
| | | ↑ADCC | 2000; Idusogie |
| | | | et al., 2001; |
| | | | Zalevsky et al., |
| | | | 2010) |
| EFTEA | G236A/S267E/H268F/S324T/ | ↑ ADCC, | (Moore et al., |
| | I332E | ↑ADCP, | 2010) |
| | | ↑ADCD | |
| EFTEALS | G236A/S267E/H268F/S324T/ | ↑ ADCC, | (Moore et al., |
| | I332E/M428L/N434S | ↑ADCP, | 2010; Zalevsky |
| | | ↑ADCD | et al., 2010) |
| | | ↑ FcRn | |
| AAA | T307A/E380A/N434A | ↑ FcRn | (Shields et al., |
| | | | 2001) |
| EANA | E380A/N434A | ↑ FcRn | (Shields et al., |
| | | | 2001) |
| N434W | N434W | ↑ FcRn | (Yeung et al., |
| | | | 2009) |
| LS | M428L/N434S | ↑ FcRn | (Zalevsky et al., |
| | | | 2010) |
| YTE | M252Y/S254T/T256E | ↓ADCC, ↑ | (Dall′Acqua et |
| | | FcRn | al., 2002) |
| YTELS | M252Y/S254T/T256E/M428L/ | ↓ADCC, ↑ | (Dall′Acqua et |
| | N434S | FcRn | al., 2002; |
| | | | Zalevsky et al., |
| | | | 2010) |
| DVNH | D376V/N434H | ↑ FcRn | (Datta-Mannan |
| | | | et al., 2007) |
| N434A | N434A | ↑ FcRn | (Shields et al., |
| | | | 2001) |
| PINH | P257I/N434H | ↑ FcRn | (Datta-Mannan |
| | | | et al., 2007) |
| PIQI | P257I/Q311I | ↑ FcRn | (Datta-Mannan |
| | | | et al., 2007) |
| PIQILS | P257I/Q311I/M428L/N434S | ↑ FcRn | (Zalevsky et al., |
| | | | 2010) |
| QL | T250Q/M428L | ↑ FcRn | (Hinton et al., |
| | | | 2004) |
| AALS | T307A/E380A/M428L/N434S | ↑ FcRn | (Zalevsky et al., |
| | | | 2010) |
| P257ILS | P257I/M428L/N434S | ↑ FcRn | (Zalevsky et al., |
| | | | 2010) |
| QLS | T250Q/M428L/N434S | ↑ FcRn | (Zalevsky et al., |
| | | | 2010) |
| ALS | E333A/M428L/N434S | ↑ ADCD, | (Zalevsky et al., |
| | | ↑ADCC, ↑ | 2010) |
| | | FcRn | |
| SELF | S267E/L328F | ↑ FcγRIIb | (Chu et al., |
| | | binding | 2008) |
| SELFLS | S267E/L328F/M428L/N434S | ↑ FcγRIIb | (Chu et al., |
| | | binding | 2008; Zalevsky |
| | | | et al., 2010) |
| D376VLS | D376V/M428L/N434S | ↑ FcRn | (Datta-Mannan |
| | | | et al., 2007; |
| | | | Zalevsky et al., |
| | | | 2010) |
| E380ALS | E380A/M428L/N434S | ↑ FcRn | (Shields et al., |
| | | | 2001; Zalevsky |
| | | | et al., 2010) |

Table 3 below provides the Fc sequences (CH2 and CH3 domains) for the REFORM variants used in this study.

TABLE 3
Fc sequences for REFORM variants

| Fc Variant | SEQ ID NO: | Sequence |
|---|---|---|
| IgG1 | SEQ ID NO: 33 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VAWESNGQPENNYKTTPPVLDSDGSFFLYSKLTVDKSR |
| | | WQQGNVFSCSVLHEALHSHYTQKSLSLSPGK |
| LALA | SEQ ID NO: 34 | PCPAPEAAGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| LALALS | SEQ ID NO: 35 | PCPAPEAAGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| LALAPG | SEQ ID NO: 36 | PCPAPEAAGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALGAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| N297Q | SEQ ID NO: 37 | PCPAPELLGGPSVFLFPPKPKDTLMISRTPEVTCVVVDV |
| | | SHEDPEVKFNWYVDGVEVHNAKTKPREEQYQSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| N297QLS | SEQ ID NO: 38 | PCPAPELLGGPSVFLFPPKPKDTLMISRTPEVTCVVVDV |
| | | SHEDPEVKFNWYVDGVEVHNAKTKPREEQYQSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPG |
| SAEAKA | SEQ ID NO: 39 | PCPAPELLGGPSVFLFPPKPKDTLMISRTPEVTCVVVDV |
| | | SHEDPEVKFNWYVDGVEVHNAKTKPREEQYNATYRV |
| | | VSVLTVLHQDWLNGKEYKCKVSNKALPAPIAATISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SAEAKALS | SEQ ID NO: 40 | PCPAPELLGGPSVFLFPPKPKDTLMISRTPEVTCVVVDV |
| | | SHEDPEVKFNWYVDGVEVHNAKTKPREEQYNATYRV |
| | | VSVLTVLHQDWLNGKEYKCKVSNKALPAPIAATISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| LPLIL | SEQ ID NO: 41 | PCPAPELLGGPSVFLLPPKPKDTLMISRTPEVTCVVVDV |
| | | SHEDPEVKFNWYVDGVEVHNAKTKPPEEQYNSTLRVV |
| | | SILTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPLVLDSDGSFFLYSKLTVDKSRW |
| | | QQGNVFSCSVMHEALHNHYTQKSLSLSPGK |
| LPLILLS | SEQ ID NO: 42 | PCPAPELLGGPSVFLLPPKPKDTLMISRTPEVTCVVVDV |
| | | SHEDPEVKFNWYVDGVEVHNAKTKPPEEQYNSTLRVV |
| | | SILTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPLVLDSDGSFFLYSKLTVDKSRW |
| | | QQGNVFSCSVLHEALHSHYTQKSLSLSPGK |
| E345R | SEQ ID NO: 43 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPRRPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| E345RLS | SEQ ID NO: 44 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPRRPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| HFST | SEQ ID NO: 45 | PCPAPELLGGPSVFLFPPKPKDTLMISRTPEVTCVVVDV |
| | | SFEDPEVKFNWYVDGVEVHNAKTKPREEQYNSTYRVV |
| | | SVLTVLHQDWLNGKEYKCKVTNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVMHEALHNHYTQKSLSLSPGK |
| HFSTLS | SEQ ID NO: 46 | PCPAPELLGGPSVFLFPPKPKDTLMISRTPEVTCVVVDV |
| | | SFEDPEVKFNWYVDGVEVHNAKTKPREEQYNSTYRVV |
| | | SVLTVLHQDWLNGKEYKCKVTNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVLHEALHSHYTQKSLSLSPGK |
| S324T | SEQ ID NO: 47 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVTNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| S324TLS | SEQ ID NO: 48 | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVTNKALPAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| RGY | SEQ ID NO: X49 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPRRPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHGALHNHYTQKYLSLSPGK |
| RGYLS | SEQ ID NO: 50 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPRRPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHGALHSHYTQKYLSLSPGK |
| SEHFST | SEQ ID NO: 51 | PCPAPELLGGPSVFLFPPKPKDTLMISRTPEVTCVVVDV |
| | | EFEDPEVKFNWYVDGVEVHNAKTKPREEQYNSTYRVV |
| | | SVLTVLHQDWLNGKEYKCKVTNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVMHEALHNHYTQKSLSLSPGK |
| SEHFSTLS | SEQ ID NO: 52 | PCPAPELLGGPSVFLFPPKPKDTLMISRTPEVTCVVVDV |
| | | EFEDPEVKFNWYVDGVEVHNAKTKPREEQYNSTYRVV |
| | | SVLTVLHQDWLNGKEYKCKVTNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVLHEALHSHYTQKSLSLSPGK |
| KWES | SEQ ID NO: 53 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNWALPAPISKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| KWESLS | SEQ ID NO: 54 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNWALPAPISKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| E333A | SEQ ID NO: 55 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIAKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| I332E | SEQ ID NO: 56 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| I332ELS | SEQ ID NO: 57 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| IEGA | SEQ ID NO: 58 | PCPAPELLAGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| IEGALS | SEQ ID NO: 59 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIE | SEQ ID NO: 60 | PCPAPELLGGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIELS | SEQ ID NO: 61 | PCPAPELLGGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIEAL | SEQ ID NO: 62 | PCPAPELLGGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIEALLS | SEQ ID NO: 63 | PCPAPELLGGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIEALYTE | SEQ ID NO: 64 | PCPAPELLGGPDVFLFPPKPKDTLYITREPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIEALYTELS | SEQ ID NO: 65 | PCPAPELLGGPDVFLFPPKPKDTLYITREPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIEGA | SEQ ID NO: 66 | PCPAPELLAGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIEGALS | SEQ ID NO: 67 | PCPAPELLAGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIESA | SEQ ID NO: 68 | PCPAPELLGGPDVFLFPPKPKDTLMISRTPEVTCVVVDV |
| | | SHEDPEVKFNWYVDGVEVHNAKTKPREEQYNATYRV |
| | | VSVLTVLHQDWLNGKEYKCKVSNKALPAPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIESALS | SEQ ID NO: 69 | PCPAPELLAGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SDIEALGA | SEQ ID NO: 70 | PCPAPELLAGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SDIEALGALS | SEQ ID NO: 71 | PCPAPELLAGPDVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPLPEEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| K326W | SEQ ID NO: 72 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNWALPAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| K326WLS | SEQ ID NO: 73 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNWALPAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| EFTEA | SEQ ID NO: 74 | PCPAPELLAGPSVFLFPPKPKDTLMISRTPEVTCVVVDV |
| | | EFEDPEVKFNWYVDGVEVHNAKTKPREEQYNSTYRVV |
| | | SVLTVLHQDWLNGKEYKCKVINKALPAPEEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| EFTEALS | SEQ ID NO: 75 | PCPAPELLAGPSVFLFPPKPKDTLMISRTPEVTCVVVDV |
| | | EFEDPEVKFNWYVDGVEVHNAKTKPREEQYNSTYRVV |
| | | SVLTVLHQDWLNGKEYKCKVINKALPAPEEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| AAA | SEQ ID NO: 76 | PCPAPELLGGPSVFLFPPKPKDTLMISRTPEVTCVVVDV |
| | | SHEDPEVKFNWYVDGVEVHNAKTKPREEQYNSTYRV |
| | | VSVLAVLHQDWLNGKEYKCKVSNKALPAPIEKTISKA |
| | | KGQPREPQVYTLPPSREEMTKNQVSLTCLVKGFYPSDI |
| | | AVAWESNGQPENNYKTTPPVLDSDGSFFLYSKLTVDKS |
| | | RWQQGNVFSCSVMHEALHAHYTQKSLSLSPGK |
| EANA | SEQ ID NO: 77 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VAWESNGQPENNYKTTPPVLDSDGSFFLYSKLTVDKSR |
| | | WQQGNVFSCSVMHEALHAHYTQKSLSLSPGK |
| N434W | SEQ ID NO: 78 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHWHYTQKSLSLSPGK |
| LS | SEQ ID NO: 79 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| YTE | SEQ ID NO: 80 | PCPAPELLGGPSVFLFPPKPKDTLYITREPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| YTELS | SEQ ID NO: 81 | PCPAPELLGGPSVFLFPPKPKDTLYITREPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| DVNH | SEQ ID NO: 82 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSVIA |
| | | VEWESNGQPENNYKTTPPVLDSDGSFFLYSKLTVDKSR |
| | | WQQGNVFSCSVMHEALHHHYTQKSLSLSPGK |
| N434A | SEQ ID NO: 83 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHAHYTQKSLSLSPGK |
| PINH | SEQ ID NO: 84 | PCPAPELLGGPSVFLFPPKPKDTLMISRTIEVTCVVVDVS |
| | | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVMHEALHHHYTQKSLSLSPGK |
| PIQI | SEQ ID NO: 85 | PCPAPELLGGPSVFLFPPKPKDTLMISRTIEVTCVVVDVS |
| | | HEDPEVKFNWYVDGVEVHNAKTKPREEQYNSTYRVV |
| | | SVLTVLHIDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVMHEALHNHYTQKSLSLSPGK |
| PIQILS | SEQ ID NO: 86 | PCPAPELLGGPSVFLFPPKPKDTLMISRTIEVTCVVVDVS |
| | | HEDPEVKFNWYVDGVEVHNAKTKPREEQYNSTYRVV |
| | | SVLTVLHIDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVLHEALHSHYTQKSLSLSPGK |
| QL | SEQ ID NO: 87 | PCPAPELLGGPSVFLFPPKPKDQLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHNHYTQKSLSLSPGK |
| AALS | SEQ ID NO: 88 | PCPAPELLGGPSVFLFPPKPKDTLMISRTPEVTCVVVDV |
| | | SHEDPEVKFNWYVDGVEVHNAKTKPREEQYNSTYRV |
| | | VSVLAVLHQDWLNGKEYKCKVSNKALPAPIEKTISKA |
| | | KGQPREPQVYTLPPSREEMTKNQVSLTCLVKGFYPSDI |
| | | AVAWESNGQPENNYKTTPPVLDSDGSFFLYSKLTVDKS |
| | | RWQQGNVFSCSVLHEALHSHYTQKSLSLSPGK |
| P257ILS | SEQ ID NO: 89 | PCPAPELLGGPSVFLFPPKPKDTLMISRTIEVTCVVVDVS |
| | | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRW |
| | | QQGNVFSCSVLHEALHSHYTQKSLSLSPGK |
| QLS | SEQ ID NO: 90 | PCPAPELLGGPSVFLFPPKPKDQLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| ALS | SEQ ID NO: 91 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIAKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVLHEALHSHYTQKSLSLSPGK |
| SELF | SEQ ID NO: 92 | PCPAPELLGGPSVFLFPPKPKDTLMISRTPEVTCVVVDV |
| | | EHEDPEVKFNWYVDGVEVHNAKTKPREEQYNSTYRV |
| | | VSVLTVLHQDWLNGKEYKCKVSNKAFPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SELFLS | SEQ ID NO: 93 | PCPAPELLGGPSVFLFPPKPKDTLMISRTPEVTCVVVDV |
| | | EHEDPEVKFNWYVDGVEVHNAKTKPREEQYNSTYRV |
| | | VSVLTVLHQDWLNGKEYKCKVSNKAFPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVLHEALHSHYTQKSLSLSPGK |
| D376VLS | SEQ ID NO: 94 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSVIA |
| | | VEWESNGQPENNYKTTPPVLDSDGSFFLYSKLTVDKSR |
| | | WQQGNVFSCSVLHEALHSHYTQKSLSLSPGK |
| E380ALS | SEQ ID NO: 95 | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VAWESNGQPENNYKTTPPVLDSDGSFFLYSKLTVDKSR |
| | | WQQGNVFSCSVLHEALHSHYTQKSLSLSPGK |

The Vic16 antibody heavy and light chain amino acid sequences used in the study as shown below in Table 4.

TABLE 4
Vic16 variants heavy and light chains

| Fc | SEQ | |
|---|---|---|
| | ID | |
| Variant | NO: | Sequence |
| VIC16- | SEQ | QVQFQQSGAELAKLGASVKMSCKASGYTFTK |
| IgG1 | ID | YWMHWIKQRPGQGLEWIGYINPSTGYSENNQ |
| | NO: | KFKGKAILTADKSSSTAYMQLSSLTSDDSAVY |
| | 96 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| DVNH | ID | YWMHWIKQRPGQGLEWIGYINPSTGYSENNQ |
| | NO: | KFKGKAILTADKSSSTAYMQLSSLTSDDSAVY |
| | 97 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSVIAVEWE |
| | | SNGQPENNYKTTPPVLDSDGSFFLYSKLTVDKS |
| | | RWQQGNVFSCSVMHEALHHHYTQKSLSLSPG |
| | | K |
| VIC16- | SEQ | QVQFQQSGAELAKLGASVKMSCKASGYTFTK |
| E345R | ID | YWMHWIKQRPGQGLEWIGYINPSTGYSENNQ |
| | NO: | KFKGKAILTADKSSSTAYMQLSSLTSDDSAVY |
| | 98 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPRRPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| EFTEA | ID | YWMHWIKQRPGQGLEWIGYINPSTGYSENNQ |
| | NO: | KFKGKAILTADKSSSTAYMQLSSLTSDDSAVY |
| | 99 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPSVFLFPPKPKDTLMIS |
| | | RTPEVTCVVVDVEFEDPEVKFNWYVDGVEVH |
| | | NAKTKPREEQYNSTYRVVSVLTVLHQDWLNG |
| | | KEYKCKVTNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| EFTEA- | ID | YWMHWIKQRPGQGLEWIGYINPSTGYSENNQ |
| LS | NO: | KFKGKAILTADKSSSTAYMQLSSLTSDDSAVY |
| | 100 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPSVFLFPPKPKDTLMIS |
| | | RTPEVTCVVVDVEFEDPEVKFNWYVDGVEVH |
| | | NAKTKPREEQYNSTYRVVSVLTVLHQDWLNG |
| | | KEYKCKVINKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| HFST | ID | YWMHWIKQRPGQGLEWIGYINPSTGYSENNQ |
| | NO: | KFKGKAILTADKSSSTAYMQLSSLTSDDSAVY |
| | 101 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSFEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| S324T | ID | YWMHWIKQRPGQGLEWIGYINPSTGYSENNQ |
| | NO: | KFKGKAILTADKSSSTAYMQLSSLTSDDSAVY |
| | 102 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| 1332E | ID | YWMHWIKQRPGQGLEWIGYINPSTGYSENNQ |
| | NO: | KFKGKAILTADKSSSTAYMQLSSLTSDDSAVY |
| | 103 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| IEGA | ID | YWMHWIKQRPGQGLEWIGYINPSTGYSENNQ |
| | NO: | KFKGKAILTADKSSSTAYMQLSSLTSDDSAVY |
| | 104 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| LALA | ID | YWMHWIKQRPGQGLEWIGYINPSTGYSENNQ |
| | NO: | KFKGKAILTADKSSSTAYMQLSSLTSDDSAVY |
| | 105 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPEAAGGPSVFLFPPKPKDTLMI |
| | | SRTPEVTCVVVDVSHEDPEVKFNWYVDGVEV |
| | | HNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| MLNS | ID | YWMHWIKQRPGQGLEWIGYINPSTGYSENNQ |
| | NO: | KFKGKAILTADKSSSTAYMQLSSLTSDDSAVY |
| | 106 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| N297Q | ID | YWMHWIKQRPGQGLEWIGYINPSTGYSENNQ |
| | NO: | KFKGKAILTADKSSSTAYMQLSSLTSDDSAVY |
| | 107 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYQSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| N434A | ID | YWMHWIKQRPGQGLEWIGYINPSTGYSENNQ |
| | NO: | KFKGKAILTADKSSSTAYMQLSSLTSDDSAVY |
| | 108 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHAHYTQKSLSLSPG |
| | | K |
| VIC16- | SEQ | QVQFQQSGAELAKLGASVKMSCKASGYTFTK |
| N434W | ID | YWMHWIKQRPGQGLEWIGYINPSTGYSENNQ |
| | NO: | KFKGKAILTADKSSSTAYMQLSSLTSDDSAVY |
| | 109 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHWHYTQKSLSLSPG |
| | | K |
| VIC16- | SEQ | QVQFQQSGAELAKLGASVKMSCKASGYTFTK |
| PIQI | ID | YWMHWIKQRPGQGLEWIGYINPSTGYSENNQ |
| | NO: | KFKGKAILTADKSSSTAYMQLSSLTSDDSAVY |
| | 110 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTIEVTCVVVDVSHEDPEVKFNWYVDGVEVH |
| | | NAKTKPREEQYNSTYRVVSVLTVLHIDWLNGK |
| | | EYKCKVSNKALPAPIEKTISKAKGQPREPQVYT |
| | | LPPSREEMTKNQVSLTCLVKGFYPSDIAVEWES |
| | | NGQPENNYKTTPPVLDSDGSFFLYSKLTVDKSR |
| | | WQQGNVFSCSVMHEALHNHYTQKSLSLSPGK |
| VIC16- | SEQ | QVQFQQSGAELAKLGASVKMSCKASGYTFTK |
| QL | ID | YWMHWIKQRPGQGLEWIGYINPSTGYSENNQ |
| | NO: | KFKGKAILTADKSSSTAYMQLSSLTSDDSAVY |
| | 111 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDQLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHNHYTQKSLSLSPGK |
| VIC16- | SEQ | QVQFQQSGAELAKLGASVKMSCKASGYTFTK |
| RGY | ID | YWMHWIKQRPGQGLEWIGYINPSTGYSENNQ |
| | NO: | KFKGKAILTADKSSSTAYMQLSSLTSDDSAVY |
| | 112 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPRRPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHGALHNHYTQKYLSLSPG |
| | | K |
| VIC16- | SEQ | QVQFQQSGAELAKLGASVKMSCKASGYTFTK |
| SDIE | ID | YWMHWIKQRPGQGLEWIGYINPSTGYSENNQ |
| | NO: | KFKGKAILTADKSSSTAYMQLSSLTSDDSAVY |
| | 113 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SDIEAL | ID | YWMHWIKQRPGQGLEWIGYINPSTGYSENNQ |
| | NO: | KFKGKAILTADKSSSTAYMQLSSLTSDDSAVY |
| | 114 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SDIEGA | ID | YWMHWIKQRPGQGLEWIGYINPSTGYSENNQ |
| | NO: | KFKGKAILTADKSSSTAYMQLSSLTSDDSAVY |
| | 115 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SDIESA | ID | YWMHWIKQRPGQGLEWIGYINPSTGYSENNQ |
| | NO: | KFKGKAILTADKSSSTAYMQLSSLTSDDSAVY |
| | 116 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNATYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SEHFST | ID | YWMHWIKQRPGQGLEWIGYINPSTGYSENNQ |
| | NO: | KFKGKAILTADKSSSTAYMQLSSLTSDDSAVY |
| | 117 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVEFEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| YTE | ID | YWMHWIKQRPGQGLEWIGYINPSTGYSENNQ |
| | NO: | KFKGKAILTADKSSSTAYMQLSSLTSDDSAVY |
| | 118 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLYIT |
| | | REPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| AA-LS | ID | YWMHWIKQRPGQGLEWIGYINPSTGYSENNQ |
| | NO: | KFKGKAILTADKSSSTAYMQLSSLTSDDSAVY |
| | 119 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLAVLHQDWLNG |
| | | KEYKCKVSNKALPAPIEKTISKAKGQPREPQVY |
| | | TLPPSREEMTKNQVSLTCLVKGFYPSDIAVAW |
| | | ESNGQPENNYKTTPPVLDSDGSFFLYSKLTVDK |
| | | SRWQQGNVFSCSVLHEALHSHYTQKSLSLSPG |
| | | K |
| VIC16- | SEQ | QVQFQQSGAELAKLGASVKMSCKASGYTFTK |
| D376V- | ID | YWMHWIKQRPGQGLEWIGYINPSTGYSENNQ |
| LS | NO: | KFKGKAILTADKSSSTAYMQLSSLTSDDSAVY |
| | 120 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSVIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| E345R- | ID | YWMHWIKQRPGQGLEWIGYINPSTGYSENNQ |
| LS | NO: | KFKGKAILTADKSSSTAYMQLSSLTSDDSAVY |
| | 121 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPRRPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| HFST- | ID | YWMHWIKQRPGQGLEWIGYINPSTGYSENNQ |
| LS | NO: | KFKGKAILTADKSSSTAYMQLSSLTSDDSAVY |
| | 122 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSFEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| 1332E- | ID | YWMHWIKQRPGQGLEWIGYINPSTGYSENNQ |
| LS | NO: | KFKGKAILTADKSSSTAYMQLSSLTSDDSAVY |
| | 123 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| IEGA- | ID | YWMHWIKQRPGQGLEWIGYINPSTGYSENNQ |
| LS | NO: | KFKGKAILTADKSSSTAYMQLSSLTSDDSAVY |
| | 124 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| LALA-LS | ID | YWMHWIKQRPGQGLEWIGYINPSTGYSENNQ |
| | NO: | KFKGKAILTADKSSSTAYMQLSSLTSDDSAVY |
| | 125 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPEAAGGPSVFLFPPKPKDTLMI |
| | | SRTPEVTCVVVDVSHEDPEVKFNWYVDGVEV |
| | | HNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| N297Q- | ID | YWMHWIKQRPGQGLEWIGYINPSTGYSENNQ |
| LS | NO: | KFKGKAILTADKSSSTAYMQLSSLTSDDSAVY |
| | 126 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYQSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| QLS | ID | YWMHWIKQRPGQGLEWIGYINPSTGYSENNQ |
| | NO: | KFKGKAILTADKSSSTAYMQLSSLTSDDSAVY |
| | 127 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDQLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| RGY-LS | ID | YWMHWIKQRPGQGLEWIGYINPSTGYSENNQ |
| | NO: | KFKGKAILTADKSSSTAYMQLSSLTSDDSAVY |
| | 128 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPRRPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHGALHSHYTQKYLSLSPGK |
| VIC16- | SEQ | QVQFQQSGAELAKLGASVKMSCKASGYTFTK |
| SDIE- | ID | YWMHWIKQRPGQGLEWIGYINPSTGYSENNQ |
| LS | NO: | KFKGKAILTADKSSSTAYMQLSSLTSDDSAVY |
| | 129 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| SDIEAL- | ID | YWMHWIKQRPGQGLEWIGYINPSTGYSENNQ |
| LS | NO: | KFKGKAILTADKSSSTAYMQLSSLTSDDSAVY |
| | 130 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| SDIEALGA | ID | YWMHWIKQRPGQGLEWIGYINPSTGYSENNQ |
| | NO: | KFKGKAILTADKSSSTAYMQLSSLTSDDSAVY |
| | 131 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SDIEALGA- | ID | YWMHWIKQRPGQGLEWIGYINPSTGYSENNQ |
| LS | NO: | KFKGKAILTADKSSSTAYMQLSSLTSDDSAVY |
| | 132 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| SDIEGA- | ID | YWMHWIKQRPGQGLEWIGYINPSTGYSENNQ |
| LS | NO: | KFKGKAILTADKSSSTAYMQLSSLTSDDSAVY |
| | 133 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLAGPDVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| SDIESA- | ID | YWMHWIKQRPGQGLEWIGYINPSTGYSENNQ |
| LS | NO: | KFKGKAILTADKSSSTAYMQLSSLTSDDSAVY |
| | 134 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLMIS |
| | | RTPEVTCVVVDVSHEDPEVKFNWYVDGVEVH |
| | | NAKTKPREEQYNATYRVVSVLTVLHQDWLNG |
| | | KEYKCKVSNKALPAPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| SEHFST- | ID | YWMHWIKQRPGQGLEWIGYINPSTGYSENNQ |
| LS | NO: | KFKGKAILTADKSSSTAYMQLSSLTSDDSAVY |
| | 135 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVEFEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| SAEAKA | ID | YWMHWIKQRPGQGLEWIGYINPSTGYSENNQ |
| | NO: | KFKGKAILTADKSSSTAYMQLSSLTSDDSAVY |
| | 136 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNATYRVVSVLTVLHQDWLNG |
| | | KEYKCKVSNKALPAPIAATISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| SDIEAL- | ID | YWMHWIKQRPGQGLEWIGYINPSTGYSENNQ |
| YTE | NO: | KFKGKAILTADKSSSTAYMQLSSLTSDDSAVY |
| | 137 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLYIT |
| | | REPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| K326W | ID | YWMHWIKQRPGQGLEWIGYINPSTGYSENNQ |
| | NO: | KFKGKAILTADKSSSTAYMQLSSLTSDDSAVY |
| | 138 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNWALPAPIEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| KWES | ID | YWMHWIKQRPGQGLEWIGYINPSTGYSENNQ |
| | NO: | KFKGKAILTADKSSSTAYMQLSSLTSDDSAVY |
| | 139 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNWALPAPISKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| LPLIL | ID | YWMHWIKQRPGQGLEWIGYINPSTGYSENNQ |
| | NO: | KFKGKAILTADKSSSTAYMQLSSLTSDDSAVY |
| | 140 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLLPPKPKDTLMIS |
| | | RTPEVTCVVVDVSHEDPEVKFNWYVDGVEVH |
| | | NAKTKPPEEQYNSTLRVVSILTVLHQDWLNGK |
| | | EYKCKVSNKALPAPIEKTISKAKGQPREPQVYT |
| | | LPPSREEMTKNQVSLTCLVKGFYPSDIAVEWES |
| | | NGQPENNYKTTPLVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| KWES- | ID | YWMHWIKQRPGQGLEWIGYINPSTGYSENNQ |
| LS | NO: | KFKGKAILTADKSSSTAYMQLSSLTSDDSAVY |
| | 141 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNWALPAPISKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| EANA | ID | YWMHWIKQRPGQGLEWIGYINPSTGYSENNQ |
| | NO: | KFKGKAILTADKSSSTAYMQLSSLTSDDSAVY |
| | 142 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVAW |
| | | ESNGQPENNYKTTPPVLDSDGSFFLYSKLTVDK |
| | | SRWQQGNVFSCSVMHEALHAHYTQKSLSLSPG |
| | | K |
| VIC16- | SEQ | QVQFQQSGAELAKLGASVKMSCKASGYTFTK |
| SELF | ID | YWMHWIKQRPGQGLEWIGYINPSTGYSENNQ |
| | NO: | KFKGKAILTADKSSSTAYMQLSSLTSDDSAVY |
| | 143 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVEHEDPEVKFNWYVDGVEVH |
| | | NAKTKPREEQYNSTYRVVSVLTVLHQDWLNG |
| | | KEYKCKVSNKAFPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| SELF- | ID | YWMHWIKQRPGQGLEWIGYINPSTGYSENNQ |
| LS | NO: | KFKGKAILTADKSSSTAYMQLSSLTSDDSAVY |
| | 144 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVEHEDPEVKFNWYVDGVEVH |
| | | NAKTKPREEQYNSTYRVVSVLTVLHQDWLNG |
| | | KEYKCKVSNKAFPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| E380A- | ID | YWMHWIKQRPGQGLEWIGYINPSTGYSENNQ |
| LS | NO: | KFKGKAILTADKSSSTAYMQLSSLTSDDSAVY |
| | 145 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVAW |
| | | ESNGQPENNYKTTPPVLDSDGSFFLYSKLTVDK |
| | | SRWQQGNVFSCSVLHEALHSHYTQKSLSLSPG |
| | | K |
| VIC16- | SEQ | QVQFQQSGAELAKLGASVKMSCKASGYTFTK |
| E333A | ID | YWMHWIKQRPGQGLEWIGYINPSTGYSENNQ |
| | NO: | KFKGKAILTADKSSSTAYMQLSSLTSDDSAVY |
| | 146 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIAKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHNHYTQKSLSLSPG |
| | | K |
| VIC16- | SEQ | QVQFQQSGAELAKLGASVKMSCKASGYTFTK |
| SAEAKA- | ID | YWMHWIKQRPGQGLEWIGYINPSTGYSENNQ |
| LS | NO: | KFKGKAILTADKSSSTAYMQLSSLTSDDSAVY |
| | 147 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNATYRVVSVLTVLHQDWLNG |
| | | KEYKCKVSNKALPAPIAATISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| LALA- | ID | YWMHWIKQRPGQGLEWIGYINPSTGYSENNQ |
| PG | NO: | KFKGKAILTADKSSSTAYMQLSSLTSDDSAVY |
| | 148 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPEAAGGPSVFLFPPKPKDTLMI |
| | | SRTPEVTCVVVDVSHEDPEVKFNWYVDGVEV |
| | | HNAKTKPREEQYNSTYRVVSVLTVLHQDWLN |
| | | GKEYKCKVSNKALGAPIEKTISKAKGQPREPQ |
| | | VYTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| LPLIL- | ID | YWMHWIKQRPGQGLEWIGYINPSTGYSENNQ |
| LS | NO: | KFKGKAILTADKSSSTAYMQLSSLTSDDSAVY |
| | 149 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLLPPKPKDTLMIS |
| | | RTPEVTCVVVDVSHEDPEVKFNWYVDGVEVH |
| | | NAKTKPPEEQYNSTLRVVSILTVLHQDWLNGK |
| | | EYKCKVSNKALPAPIEKTISKAKGQPREPQVYT |
| | | LPPSREEMTKNQVSLTCLVKGFYPSDIAVEWES |
| | | NGQPENNYKTTPLVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| S324T- | ID | YWMHWIKQRPGQGLEWIGYINPSTGYSENNQ |
| LS | NO: | KFKGKAILTADKSSSTAYMQLSSLTSDDSAVY |
| | 150 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVTNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| SDIEAL- | ID | YWMHWIKQRPGQGLEWIGYINPSTGYSENNQ |
| YTE | NO: | KFKGKAILTADKSSSTAYMQLSSLTSDDSAVY |
| | 151 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLYIT |
| | | REPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| VIC16- | SEQ | QVQFQQSGAELAKLGASVKMSCKASGYTFTK |
| SDIEAL- | ID | YWMHWIKQRPGQGLEWIGYINPSTGYSENNQ |
| YTE-LS | NO: | KFKGKAILTADKSSSTAYMQLSSLTSDDSAVY |
| | 152 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPDVFLFPPKPKDTLYIT |
| | | REPEVTCVVVDVSHEDPEVKFNWYVDGVEVH |
| | | NAKTKPREEQYNSTYRVVSVLTVLHQDWLNG |
| | | KEYKCKVSNKALPLPEEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| K326W- | ID | YWMHWIKQRPGQGLEWIGYINPSTGYSENNQ |
| LS | NO: | KFKGKAILTADKSSSTAYMQLSSLTSDDSAVY |
| | 153 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNWALPAPIEKTISKAKGQPREPQV |
| | | YTLPPSREEMTKNQVSLTCLVKGFYPSDIAVE |
| | | WESNGQPENNYKTTPPVLDSDGSFFLYSKLTV |
| | | DKSRWQQGNVFSCSVLHEALHSHYTQKSLSLS |
| | | PGK |
| VIC16- | SEQ | QVQFQQSGAELAKLGASVKMSCKASGYTFTK |
| AAA | ID | YWMHWIKQRPGQGLEWIGYINPSTGYSENNQ |
| | NO: | KFKGKAILTADKSSSTAYMQLSSLTSDDSAVY |
| | 154 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLAVLHQDWLNG |
| | | KEYKCKVSNKALPAPIEKTISKAKGQPREPQVY |
| | | TLPPSREEMTKNQVSLTCLVKGFYPSDIAVAW |
| | | ESNGQPENNYKTTPPVLDSDGSFFLYSKLTVDK |
| | | SRWQQGNVFSCSVMHEALHAHYTQKSLSLSPG |
| | | K |
| VIC16- | SEQ | QVQFQQSGAELAKLGASVKMSCKASGYTFTK |
| YTE-LS | ID | YWMHWIKQRPGQGLEWIGYINPSTGYSENNQ |
| | NO: | KFKGKAILTADKSSSTAYMQLSSLTSDDSAVY |
| | 155 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLYIT |
| | | REPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| PINH | ID | YWMHWIKQRPGQGLEWIGYINPSTGYSENNQ |
| | NO: | KFKGKAILTADKSSSTAYMQLSSLTSDDSAVY |
| | 156 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTIEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVMHEALHHHYTQKSLSLSPG |
| | | K |
| VIC16- | SEQ | QVQFQQSGAELAKLGASVKMSCKASGYTFTK |
| PIQI- | ID | YWMHWIKQRPGQGLEWIGYINPSTGYSENNQ |
| LS | NO: | KFKGKAILTADKSSSTAYMQLSSLTSDDSAVY |
| | 157 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTIEVTCVVVDVSHEDPEVKFNWYVDGVEVH |
| | | NAKTKPREEQYNSTYRVVSVLTVLHIDWLNGK |
| | | EYKCKVSNKALPAPIEKTISKAKGQPREPQVYT |
| | | LPPSREEMTKNQVSLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| VIC16- | SEQ | QVQFQQSGAELAKLGASVKMSCKASGYTFTK |
| P257I- | ID | YWMHWIKQRPGQGLEWIGYINPSTGYSENNQ |
| LS | NO: | KFKGKAILTADKSSSTAYMQLSSLTSDDSAVY |
| | 158 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTIEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | QVQFQQSGAELAKLGASVKMSCKASGYTFTK |
| ALS | ID | YWMHWIKQRPGQGLEWIGYINPSTGYSENNQ |
| | NO: | KFKGKAILTADKSSSTAYMQLSSLTSDDSAVY |
| | 159 | YCVRGYDSHYYVMDYWGQGTSVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKSC |
| | | DKTHTCPPCPAPELLGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIAKTISKAKGQPREPQVY |
| | | TLPPSREEMTKNQVSLTCLVKGFYPSDIAVEWE |
| | | SNGQPENNYKTTPPVLDSDGSFFLYSKLTVDKS |
| | | RWQQGNVFSCSVLHEALHSHYTQKSLSLSPGK |
| VIC16- | SEQ | DIVITQDTASLAVSRGQRATISCRASESVEYYG |
| LC | ID | TTLMQWYQQRPGQPPKLLIYGASNVESGVPAR |
| | NO: | FSGSGSGTDFSLNIHPVEEDDIAMYFCQQSRKV |
| | 160 | PYTFGGGTKLEIKRTVAAPSVFIFPPSDEQLKSG |
| | | TASVVCLLNNFYPREAKVQWKVDNALQSGNS |
| | | QESVTEQDSKDSTYSLSSTLTLSKADYEKHKV |
| | | YACEVTHQGLSSPVTKSFNRGEC |

In some embodiments, the effector functions of the VIC16 antibodies of the disclosure may be enhanced by at least 10%, 20%, 30%, 40%, 50%, 60%, 70%, 80%, 90%, or 95% relative to the effector functions of a wild-type VIC16 antibody. In some embodiments, the VIC16 antibody of the disclosure results in a high level of antibody-mediated complement deposition (ADCD). For instance, the ADCD activity associated with a VIC16 antibody of the disclosure may be enhanced by at least 10%, 20%, 30%, 40%, 50%, 60%, 70%, 80%, 90%, or 95% relative to that of a wild-type VIC16 antibody. In some embodiments, the VIC16 antibody of the disclosure results in high NK cell-mediated activity. For instance, the VIC16 antibody results in antibody-dependent NK cell activation (ADNKA) that is enhanced by at least 10%, 20%, 30%, 40%, 50%, 60%, 70%, 80%, 90%, or 95% relative to that of a wild-type VIC16 antibody. For example, the VIC16 antibody may enhance the activation markers CD107a, MIP1β and/or IFNγ by at least 10%, 20%, 30%, 40%, 50%, 60%, 70%, 80%, 90%, or 95% relative to that of a wild-type VIC16 antibody as determined by assays known in the art and described in this application. In some embodiments, the VIC16 antibody of the disclosure results in high antibody dependent cellular phagocytosis (ADCP), such as monocyte phagocytosis or neutrophil phagocytosis. For instance, the VIC16 antibody results in ADCP that is enhanced by at least 10%, 20%, 30%, 40%, 50%, 60%, 70%, 80%, 90%, or 95% relative to that of a wild-type VIC16 antibody. In some instances, the VIC16 antibody results in antibody dependent neutrophil phagocytosis (ADNP) that is enhanced by at least 10%, 20%, 30%, 40%, 50%, 60%, 70%, 80%, 90%, or 95% relative to that of a wild-type VIC16 antibody.

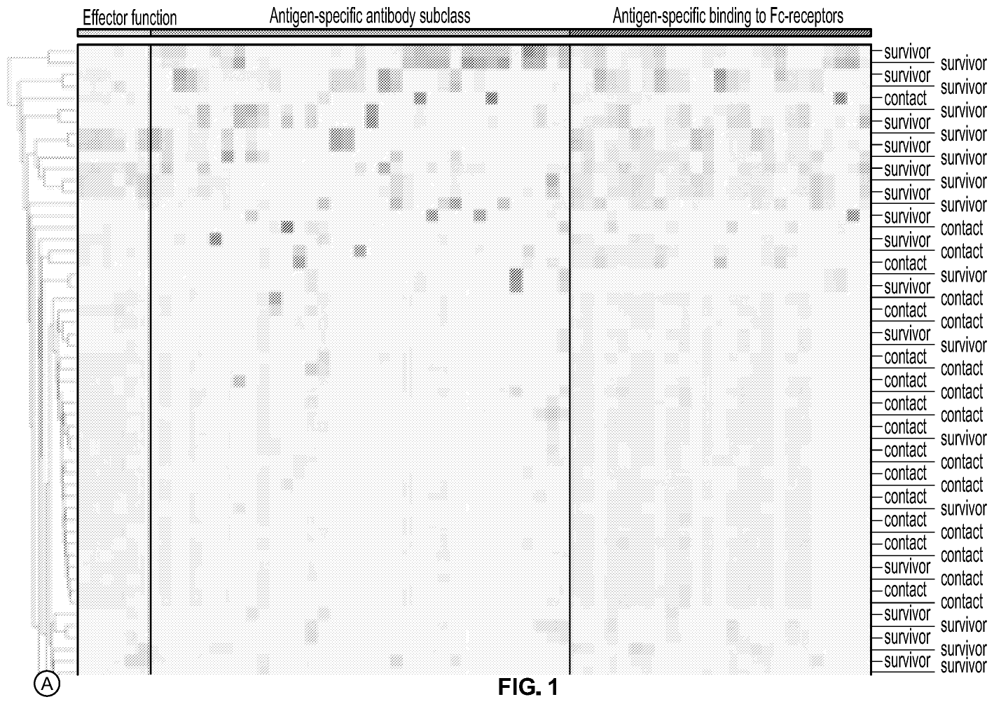

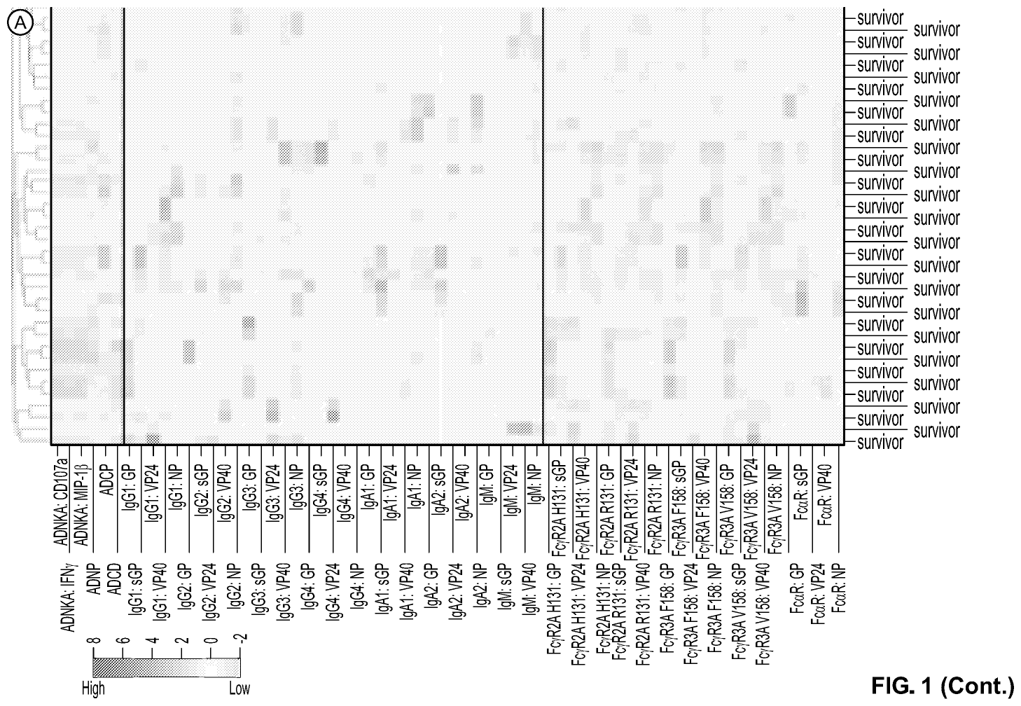

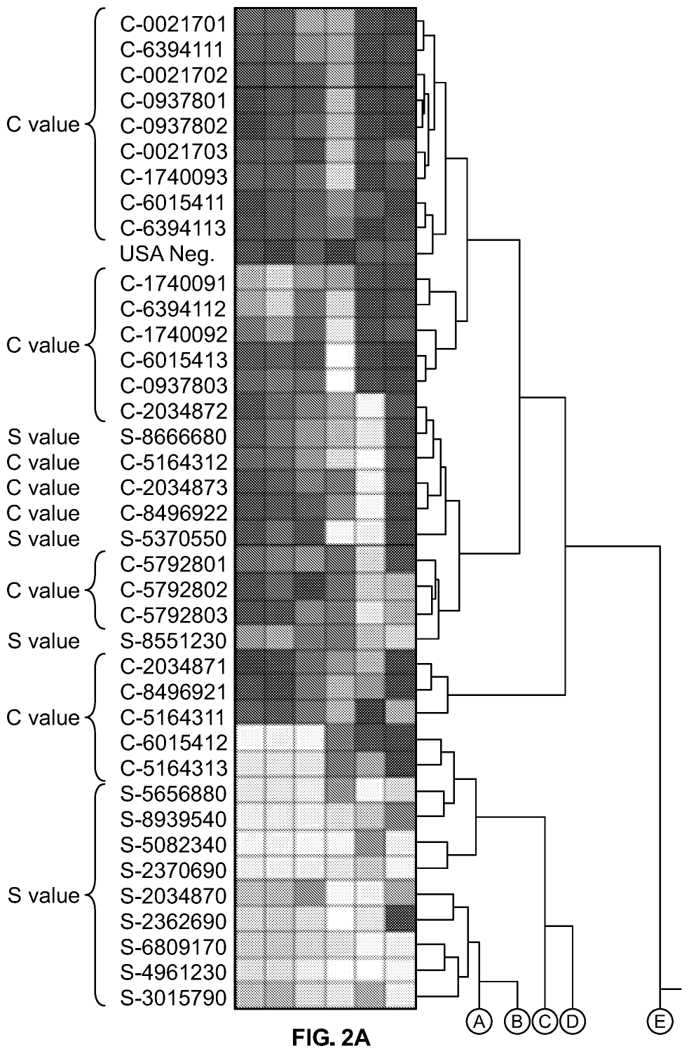

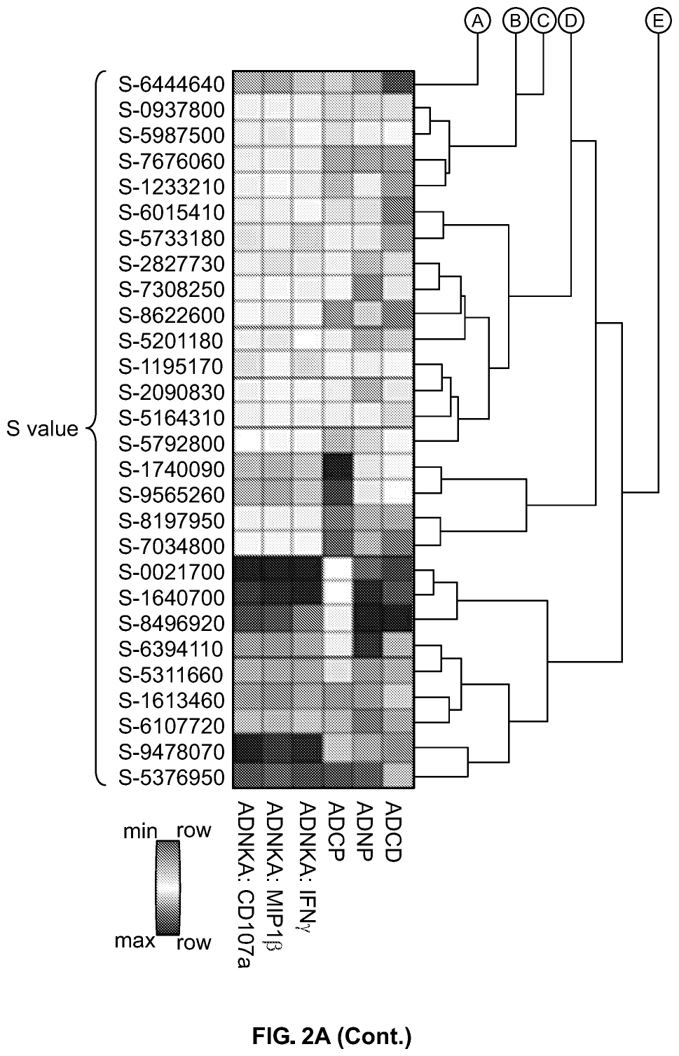

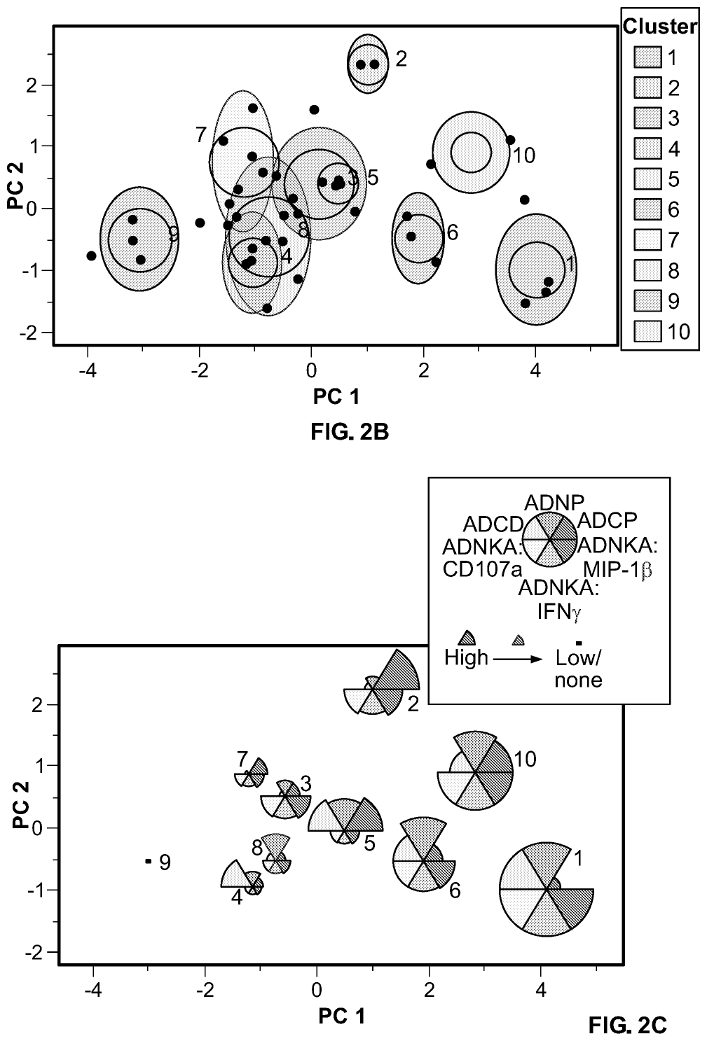

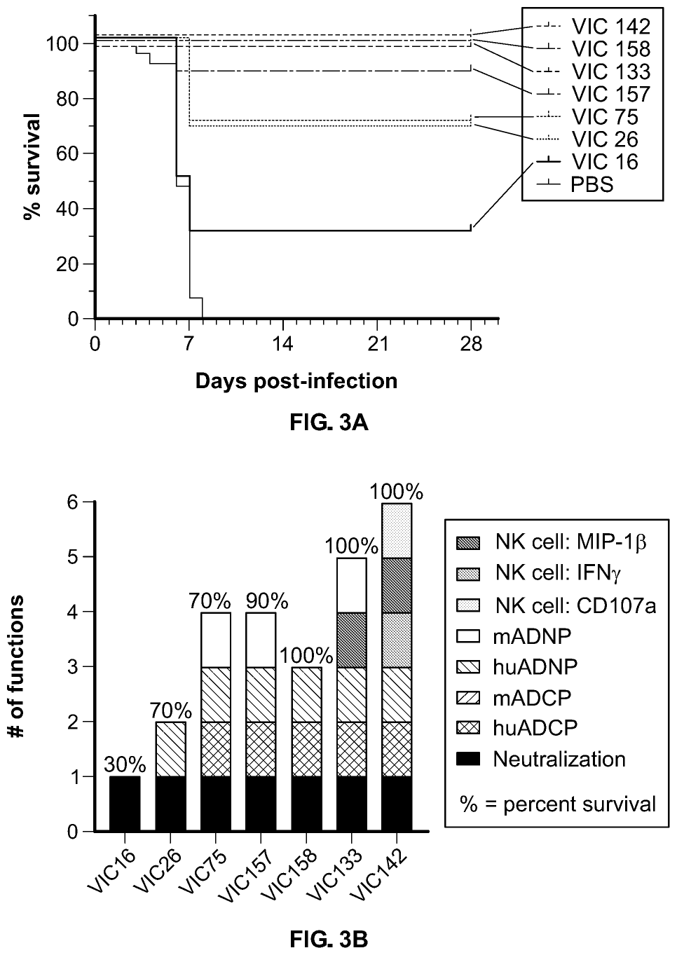

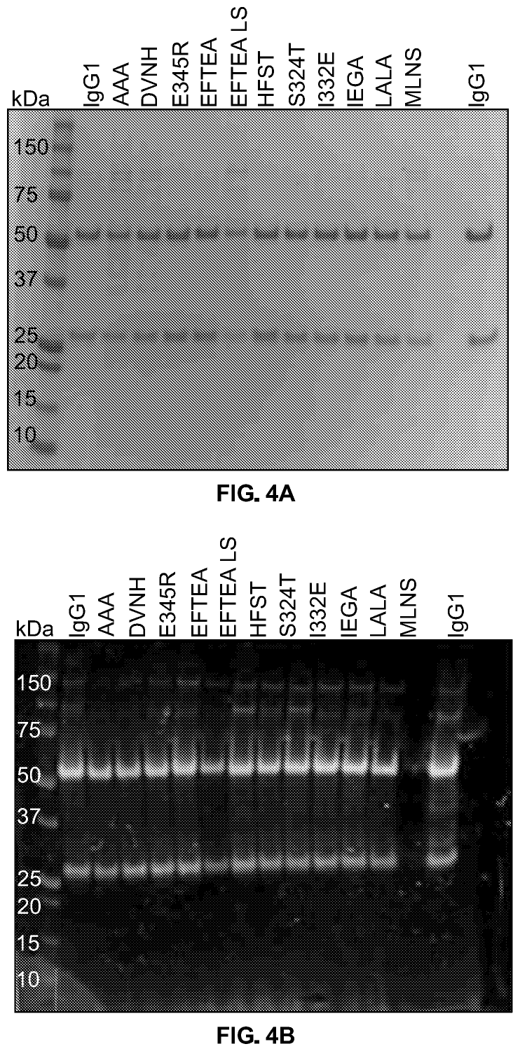

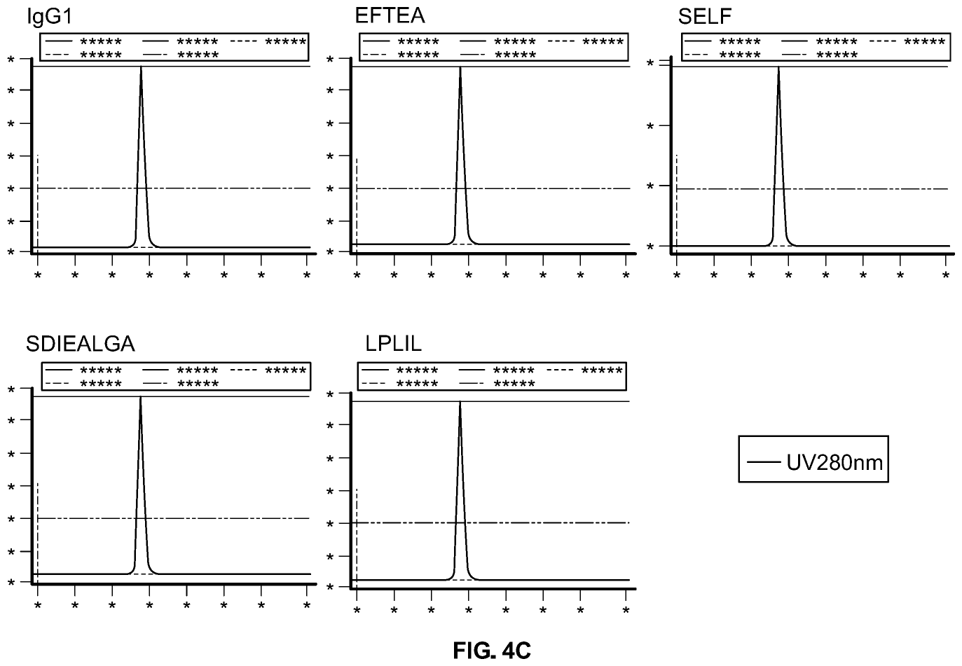

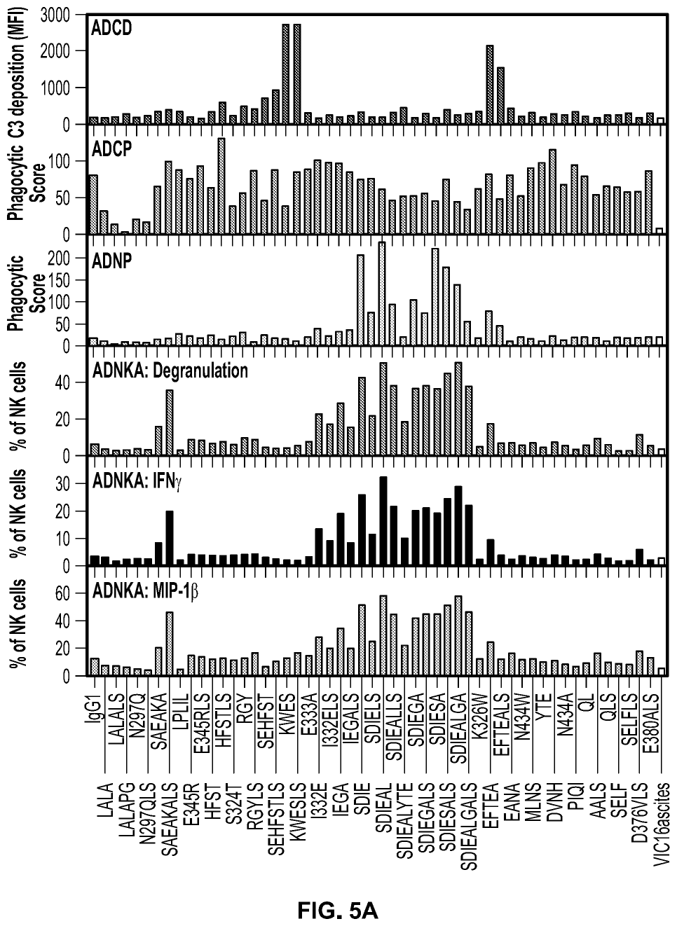

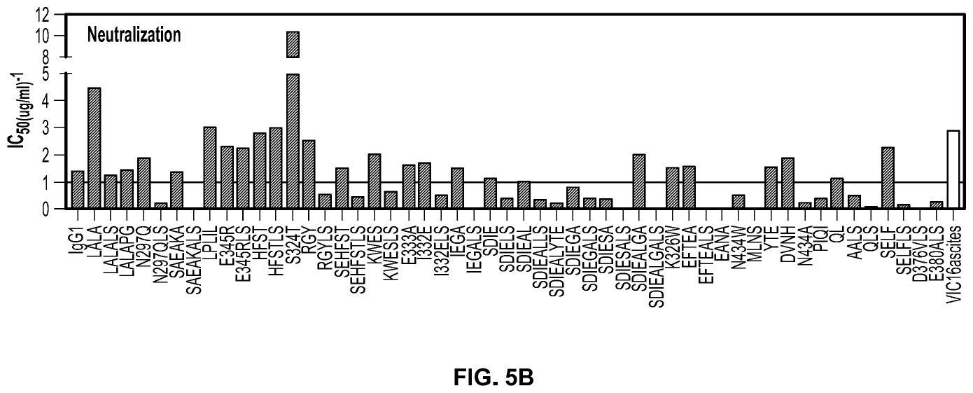

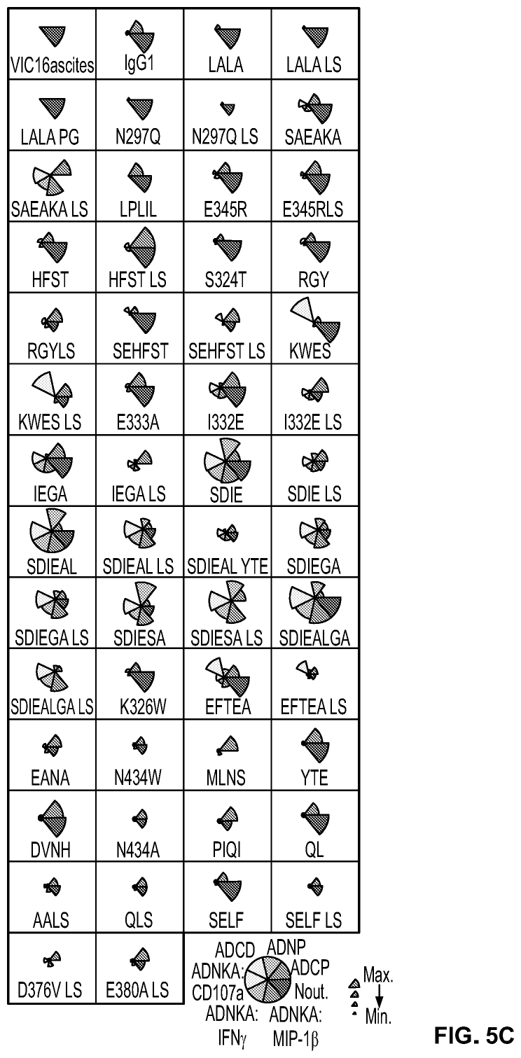

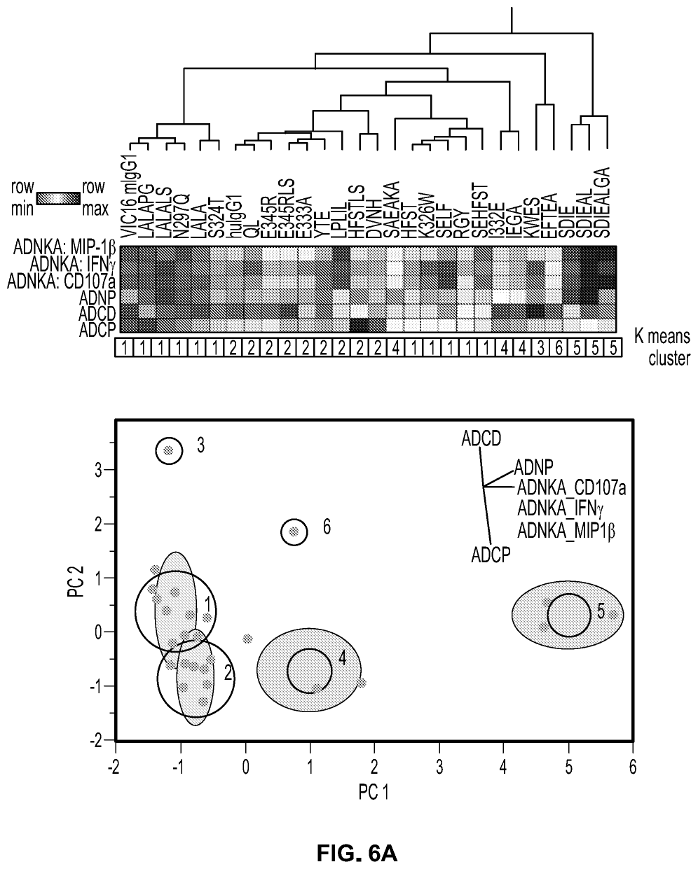

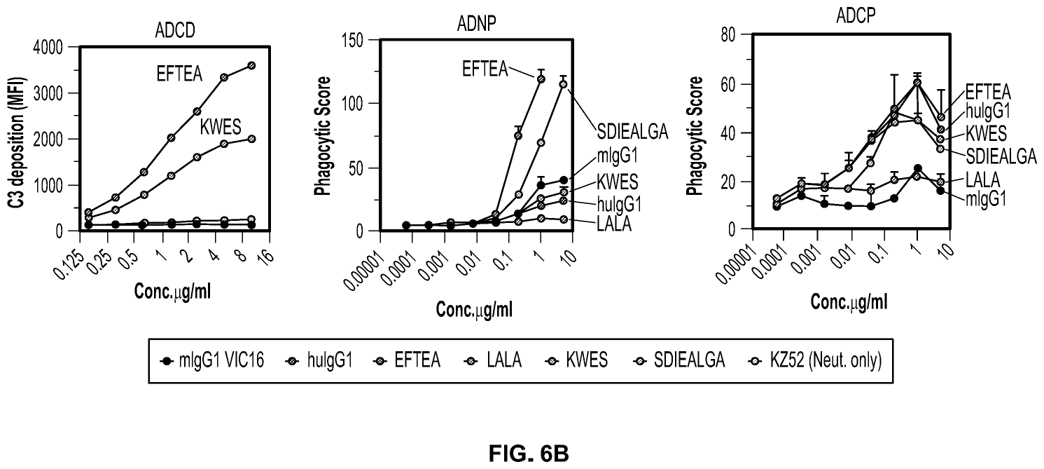

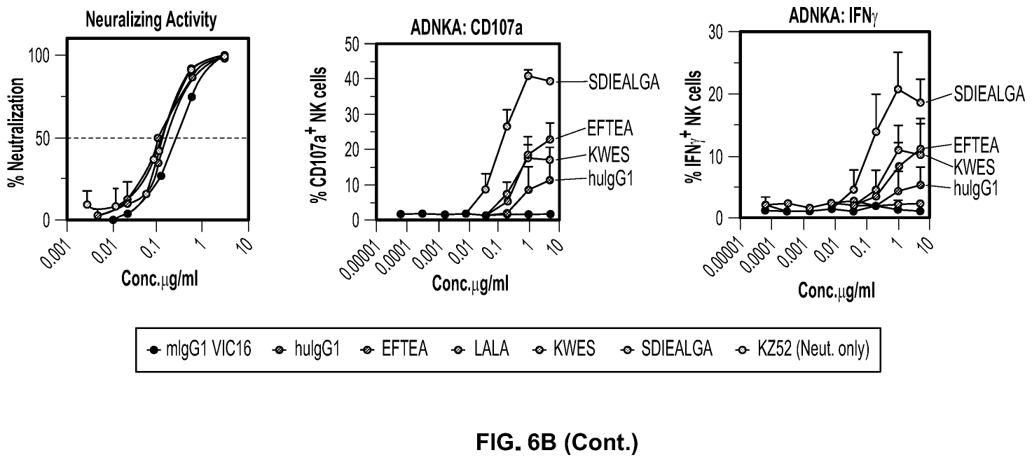

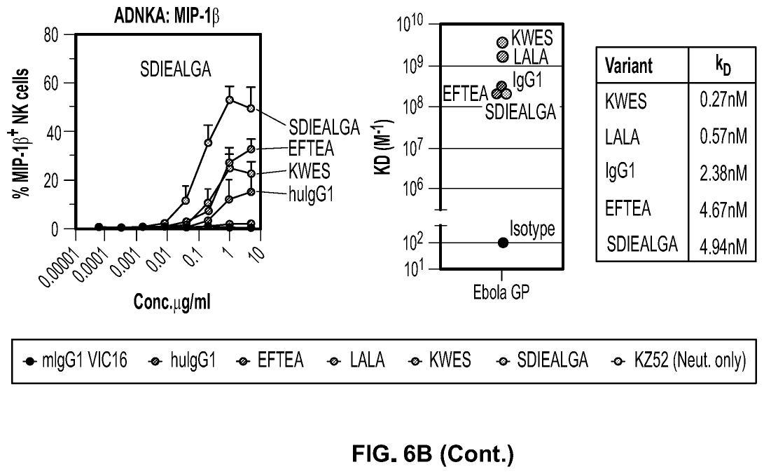

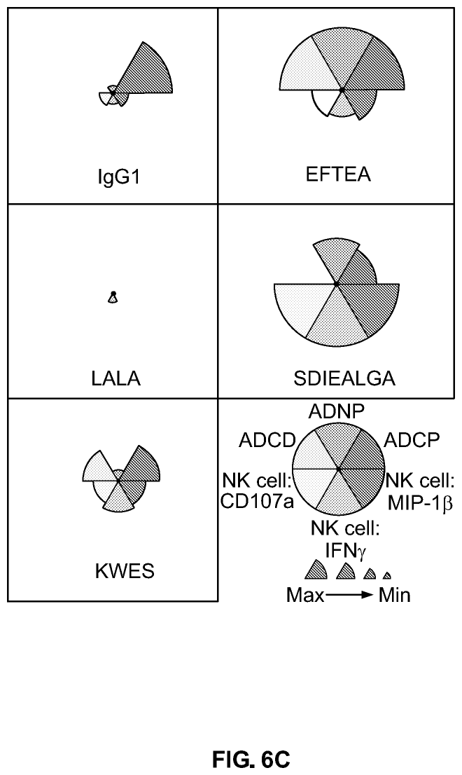

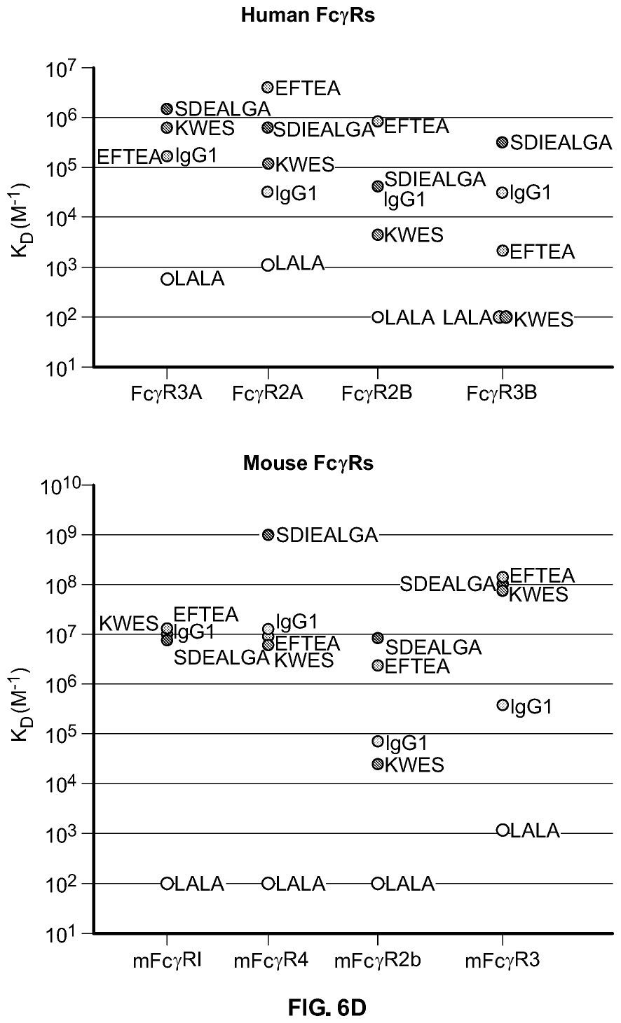

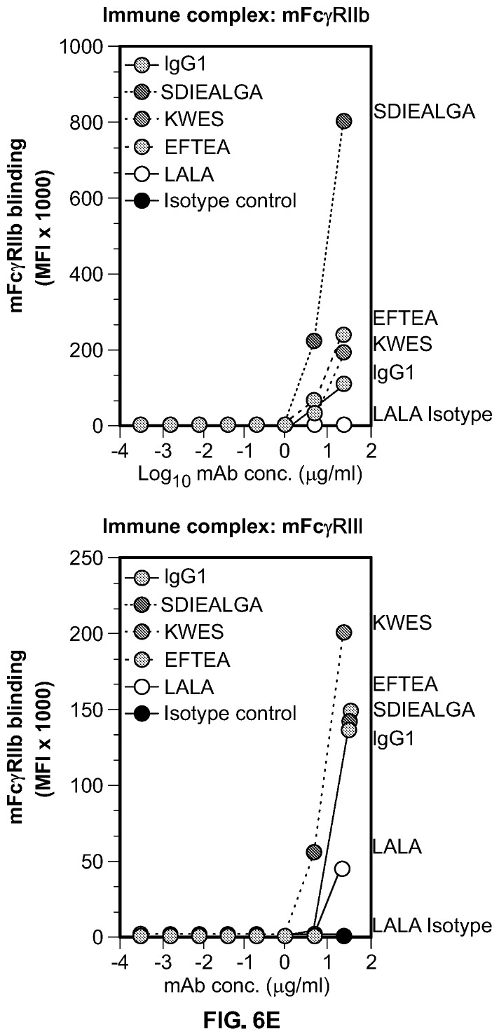

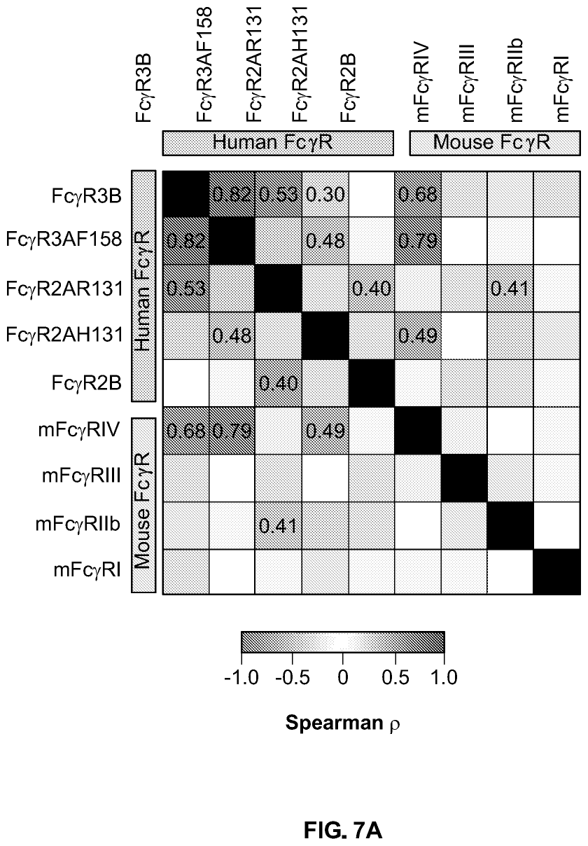

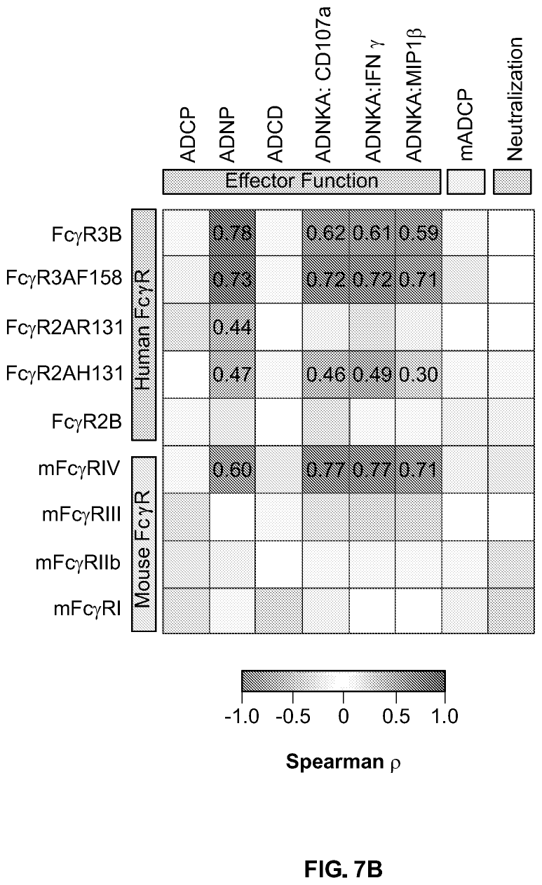

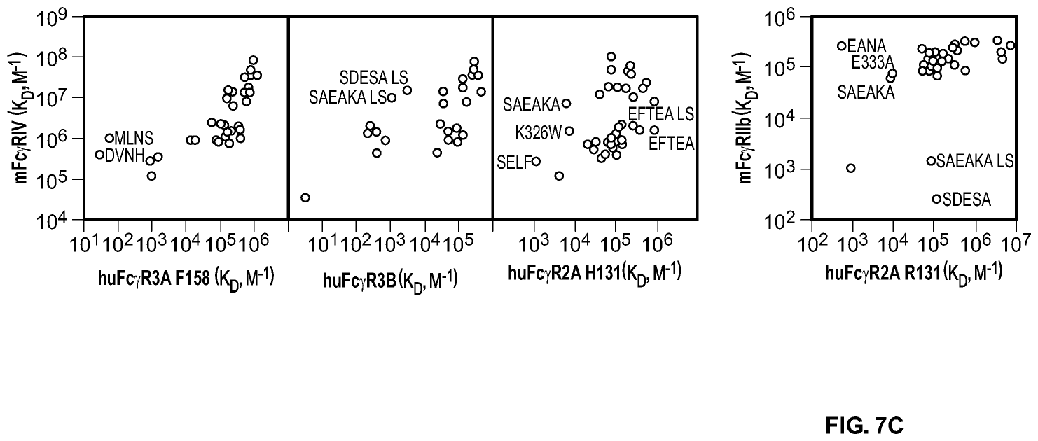

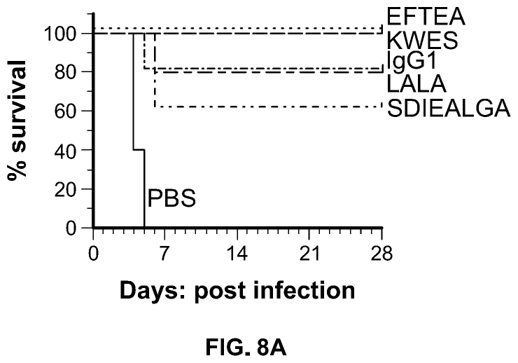

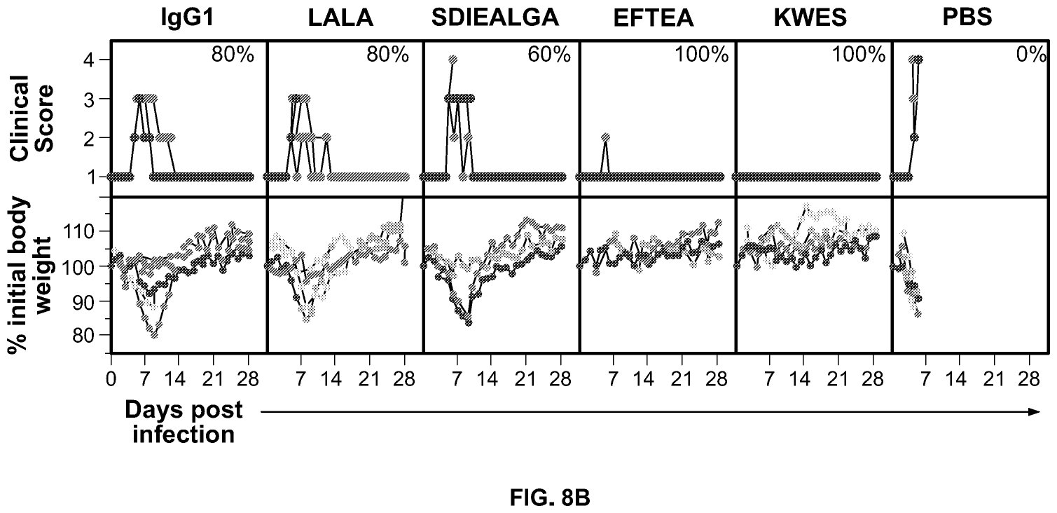

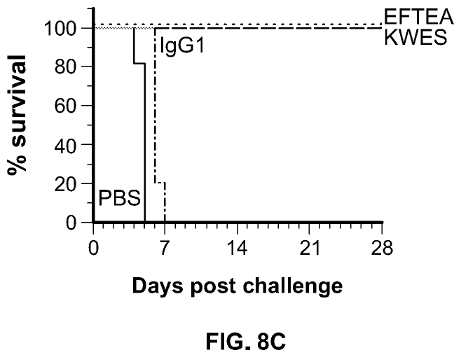

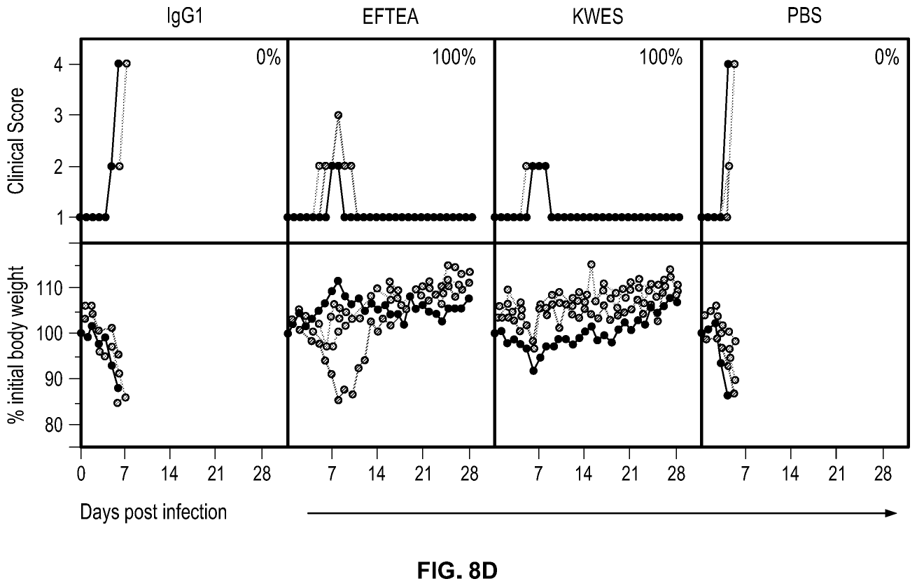

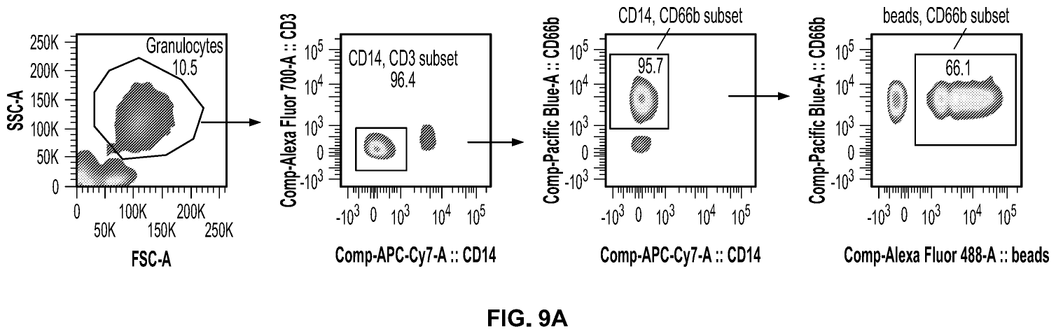

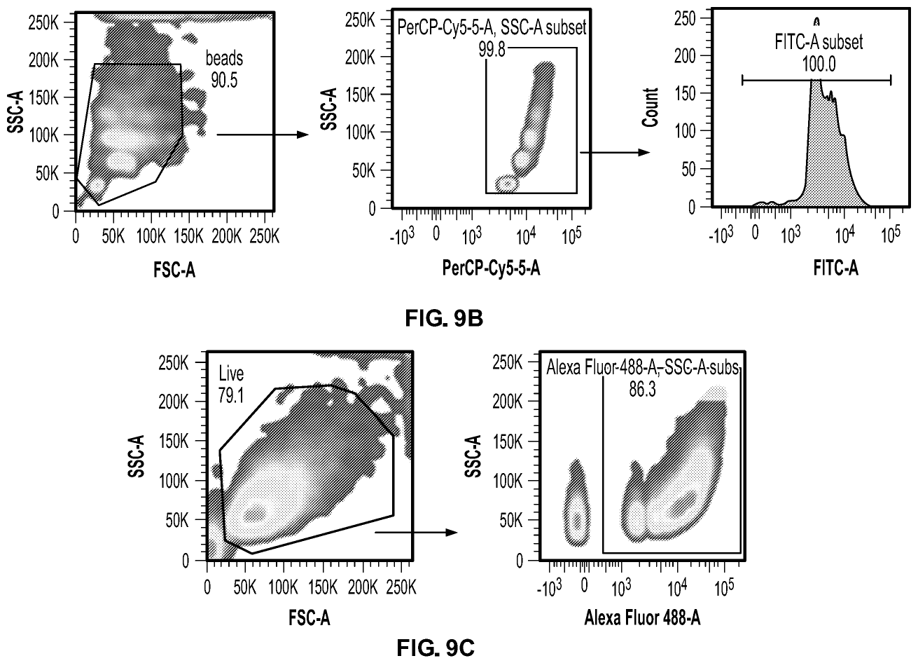

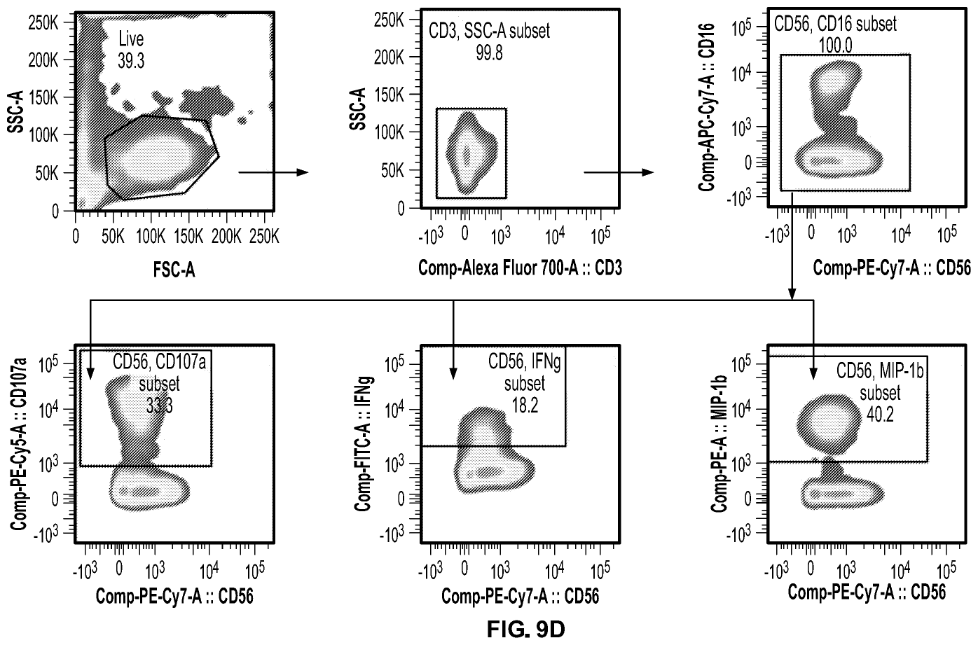

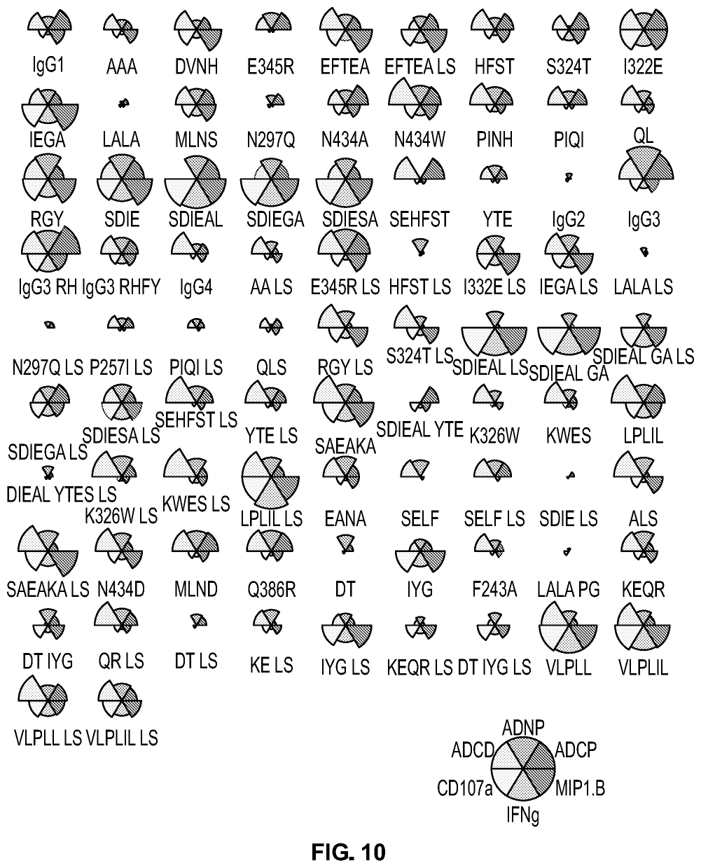

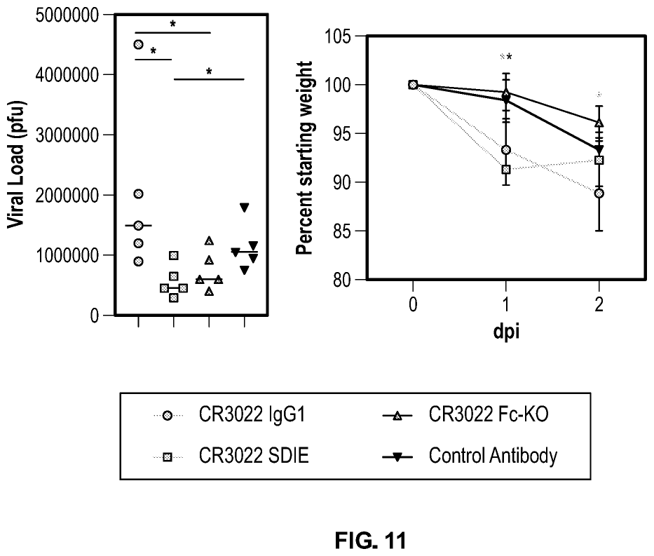

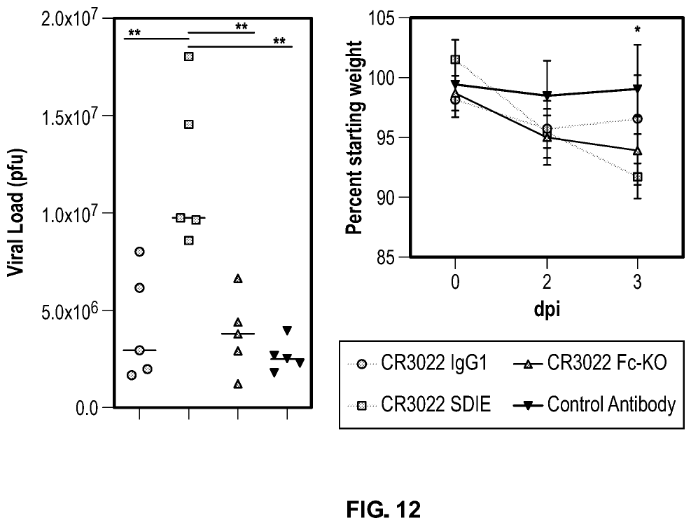

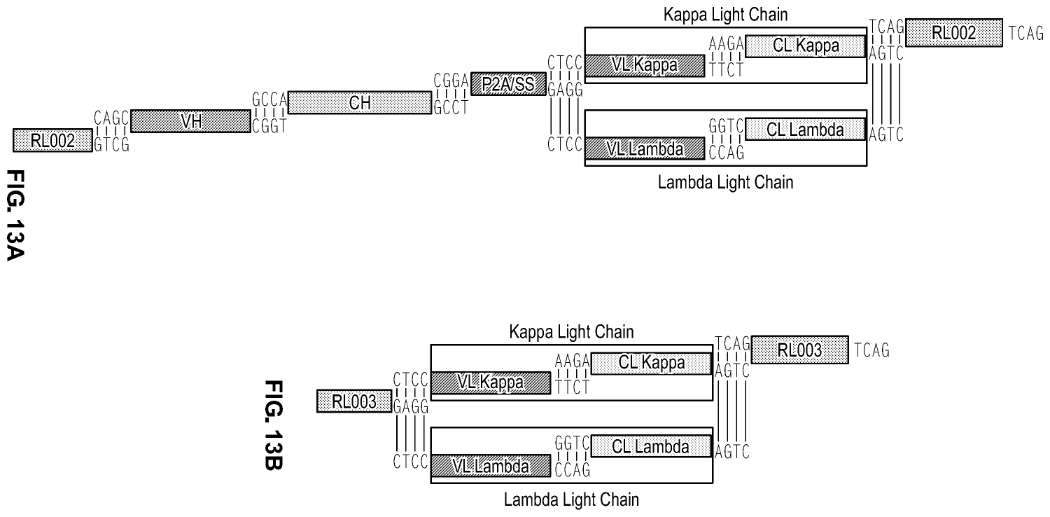

The 5 plasmids were combined in a single digestion-ligation reaction to generate an expression plasmid encoding both the heavy chain and light chain of a monoclonal antibody (FIG. 1) as follows: 50 ng of each donor and destination plasmid, 20 units BsaI-HF (NEB), 200 units T4 ligase, and 0.67 mM ATP in 1×T4 ligase buffer (NEB) supplemented with BSA were combined and incubated at 37° C. for 1 h followed by incubation at 50° C. for 15 min and 80° C. for 15 min. Ligation was performed by adding 200 units T4 ligase followed by incubation at 16° C. for 30 min. Inclusion of a ccdB gene that is removed during the digestion-ligation process increases the likelihood that the recombinant plasmid is selected as opposed to the parental vector. Two alternate destination vectors were created for expression of only the light chain to optimize the heavy chain to light chain ratio during antibody production (FIGS. 13A-13D). Ligation products (2 μl) were transformed into Stellar competent cells (Clontech), and plated onto agar plates with kanamycin. The resulting colonies were screened and sequenced to verify the identity of each new plasmid.

Production of antibodies in mammalian cells: Plasmids were expanded and transfected into 293F suspension cells grown in FreeStyle™ 293 Expression media (Gibco). Cells were seeded two days pre-transfection at a density of 0.4×10⁶ cells/mL in 50 mL in a 125 mL baffled Erlenmeyer flask with a vented closure. On the day of transfection, cells were counted again and the cell density was adjusted to 1.2×10⁶ cells/mL in 50 mL media. Total DNA (25 μg) was transfected into cells using Polyethylenimine (PEI) (Polysciences) at 1 μg/μl in a ratio of 3 μg PEI to 1 μg DNA. Cell culture supernatants were harvested 5 days post-transfection. Supernatants were incubated with Protein G magnetic beads (Millipore) overnight at 4° C. The Protein G Beads were washed 4 times with 1×PBS before antibodies were eluted using Pierce™ IgG Elution Buffer (Thermo Fisher Scientific) and neutralized with Tris-HCl pH 8.0 at a ratio of 1:10. For 500 mL production volumes, cells were transfected with 250 μg total DNA with PEI at the same ratio as that used for smaller production. Cell culture supernatants were harvested 5 days post-transfection and incubated overnight at 4° C. with Pierce™ Protein A/G Plus Agarose. Agarose resin was collected by pouring the supernatant over a BioRad Econo-Column Chromatography Column. Resin was washed with PBS before antibody was eluted with Pierce™ IgG Elution Buffer directly into Tris-HCl pH 8.0 at a ratio of 1:10. Antibody eluates were concentrated using Amicon Ultra-15 Centrifugal Filter Units with a 50 kDa molecular weight cut off to a final volume of approximately 1 mL.

Neutralizing activity: Three-fold dilutions of antibodies (3 μg/ml-0.0048 μg/ml) were assayed in technical duplicates and incubated with 1×10⁴ RLU/well of Ebola GP-pseudotyped vesicular stomatitis virus expressing luciferase (IBT Bioservices) for 1 hour at room temperature before addition to Vero-E6 monolayers (6×10⁵ cells/well) and incubation at 37° C. for 14-16 h. Cells were lysed using Passive Lysis Buffer (Promega) and luciferase activity was determined using a luciferase activating reagent (Promega). ICso titers were determined using Prism 8.0.

Antigen-binding ELISA: Recombinant Ebola GP antigen (IBT Bioservices) was coated onto MaxiSorp 384-well plates (Nunc) at 62.5 ng/well at 4° C. overnight. Wells were washed with PBS and blocked with 5% BSA prior to addition of 2-fold dilutions of antibodies (10-0.08 μg/ml) for 2 h at room temperature. Wells were washed 6 times with PBS+0.01% Tween 20 and incubated for 1 h with an HRP-conjugated anti-human IgG Fc antibody (1:5000; Jackson ImmunoResearch). Wells were again washed 6 times with PBS+0.01% Tween 20, incubated with 1×TMB substrate for 5 minutes and the reaction was stopped with 1N H₂SO₄ before absorbance at 405 nm was measured. The area under the curve was determined using Prism 8.0.

Antigen affinity measurement by biolayer inferometry: 0.3 μg/ml of each mAb was immobilized to anti-human IgG Fc (AHC) kinetic biosensors (Sartorius/ForteBio) in 1×kinetics buffer for 180s followed by association and dissociation measurements to two-fold dilutions of recombinant Ebola GP starting at 500 nM on an Octet Red96 instrument (Forte Bio). Association and dissociation were measured for 120s and 600s, respectively. Following subtraction of the reference control, the KD was calculated using a 1:1 binding model in the Octet Data Analysis software version 8.1.

Antibody-dependent NK cell degranulation: Recombinant Ebola GP antigen (IBT Bioservices) was coated onto MaxiSorp 96-well plates (Nunc) at 300 ng/well at 4° C. overnight. Wells were washed with PBS and blocked with 5% BSA prior to addition of antibodies (5 μg/ml) and incubation for 2 h at 37° C. Unbound antibodies were removed by washing with PBS, and NK cells enriched from the peripheral blood of human donors were added at 5×10⁴ cells/well in the presence of 4 μg/ml brefeldin A (Sigma-Aldrich), 5 μg/ml GolgiStop (Life Technologies) and anti-CD107a antibody (Clone H4A3, BD Biosciences) for 5 h. Cells were fixed and permeabilized with Fix/Perm (Life Technologies) according to the manufacturer's instructions to stain for intracellular IFNy (Clone B27, BD Biosciences), and MIP-1β (Clone D21-1351, BD Biosciences). Cells were analyzed on a BD LSRII flow cytometer. Gating strategy and representative flow cytometry plots are shown in FIGS. 9A-9D.

Antibody-dependent neutrophil phagocytosis (ADNP): Biotinylated Ebola GP was coupled to yellow-green Neutravidin beads (Life Technologies). Antibodies were diluted in culture medium to 5 μg/ml and incubated with GP-coated beads for 2 h at 37° C. Freshly isolated white blood cells from human donor peripheral blood (5×10⁴ cells/well) were incubated for lh at 37° C. Cells were then stained for CD66b (Clone G10F5; BioLegend), CD3 (Clone UCHT1; BD Biosciences), and CD14 (Clone MP9; BD Biosciences), fixed with 4% paraformaldehyde, and analyzed by flow cytometry. Neutrophils were defined as SSC-A^{high} CD66b⁺, CD3⁻, CD14⁻. A phagocytic score was determined using the following formula: (percentage of FITC+cells)*(geometric mean fluorescent intensity (gMFI) of the FITC+cells)/10,000. Gating strategy and representative flow cytometry plots are shown in FIGS. 9A-9D.

Antibody-dependent cellular phagocytosis by human monocytes (ADCP): GP-coated beads were generated as described for ADNP. Antibodies were diluted in culture medium to 5 μg/ml and incubated with GP-coated beads for 2 h at 37° C. Unbound antibodies were removed by centrifugation prior to the addition of THP-1 cells at 2.5×10⁴ cells/well. Cells were fixed with 4% paraformaldehyde and analyzed by flow cytometry. A phagocytic score was determined as described above. Gating strategy and representative flow cytometry plots are shown in FIGS. 9A-9D.

Antibody-mediated complement deposition (ADCD): GP-coated beads were generated as described for ADNP, but with substitution of red-fluorescent Neutravidin beads (Life Technologies). Antibodies were diluted in culture medium to 5 μg/ml and incubated with GP-coated beads for 2 h at 37° C. Unbound antibodies were removed by centrifugation prior to the addition of reconstituted guinea pig complement (Cedarlane Labs) diluted in veronal buffer supplemented with calcium and magnesium (Boston Bioproducts) for 20 min at 37° C. Beads were washed with PBS containing 15 mM EDTA, and stained with an FITC-conjugated anti-guinea pig C3 antibody (MP Biomedicals). C3 deposition onto beads was analyzed by flow cytometry. The gMFI of FITC of all beads was measured. Gating strategy and representative flow cytometry plots are shown in FIGS. 9A-9D.

Measurement of FcR binding kinetics by SPR: Each mAb (12.5 μg/ml) was printed onto a MX96 200M chip (Xantec Bioanalytics) using a continuous flow microspotter (CFM) (Wasatch Microfluidics) with sulfo-NHS and EDC coupling chemistry. Measurement of FcγR binding kinetics was performed using an image-based array reader (MX96, IBIS Technologies). The chip was quenched with 1 M ethanolamine and 3-fold dilutions of recombinant FcγR (Duke University; 10 μM to 0.005 μM) were sequentially injected. To account for non-specific signals, the signals for each antibody were double-referenced using signals from both blank injections and from uncoupled inter-spots between antibodies. Data was processed using Sprint software (IBIS Technologies), and the kinetics of binding (K_{D}) was determined using Kinetics software (Carterra).

Measurement of FcR binding kinetics by biolayer inferometry: 100 nM of each mAb was immobilized to anti-human IgG Fab-CH1 kinetic biosensors (Sartorius/ForteBio) in 1×kinetics buffer for 300s followed by association and dissociation measurements to two-fold dilutions of recombinant murine FcRs starting at 200 nM (mFcγRI and mFcγRIV) or 600 nM (mFcγRIIb and mFcγRIII) on an Octet Red96 instrument (Forte Bio). Association and dissociation were measured for 120s and 600s, respectively. Following subtraction of the reference control, the K_{D} was calculated using a 2:1 analyte binding model in the Octet Data Analysis software version 8.1.

Measurement of murine FcγR binding to immune complexes: Recombinant EBOV GP (IBT Bioservices) were coupled to MagPlex beads (Luminex) via sulfo-NHS coupling chemistry. Murine FcγR receptors (FcγRIII, FcγRIV, FcγRI) and one inhibitory receptor (FcγR2B). Serial dilutions of the indicated antibodies samples were diluted in 1×PBS+0.1% bovine serum albumin (BSA)+0.05% Tween20 and incubated with antigen-coupled beads for 2 hours. Beads were washed and incubated with recombinant biotinylated Fc-receptors that were tetramerized via Streptavidin-PE for 1 hour at room temperature. Beads were washed analyzed on a Sartorius iQue screener. The median fluorescent intensity of 30 beads/region was recorded. A human IgG1 isotype control was used to establish antigen-specificity and background binding.

In vivo protection: The animal protocol for testing of mAbs in mice was approved by the Institutional Animal Care and Use Committee of the UTMB. Seven-week-old BALB/c mice (Charles River Laboratories) were placed in the ABSL-4 facility at the Galveston National Laboratory. Groups of five animals were injected intraperitoneally with 1,000 PFU of mouse-adapted EBOV, strain Mayinga (Bray et al., 1998). The animals were injected with mAbs by the intraperitoneal route using 0.1 mg or 0.03 mg per treatment 24 h after virus challenge. Animals treated with PBS served as controls. Animal observation procedures were performed as described elsewhere (Ilinykh et al., 2018). The overall observation period was 28 days.

K-means clustering analysis: K-means clustering analysis was performed in JMP Pro 15, and elbow joint analysis was used to determine the optimal number of clusters in R.

Hierarchical clustering analysis. Unsupervised hierarchical clustering analysis was performed in JMP Pro 15, using the Ward method.

Exemplary Sequences
PALIVIZUMAB VL
(SEQ ID NO: 1)
GACATCCAGATGACACAGAGCCCCAGCACACTGTCTGCCAGCGTGGGAG
ACAGAGTGACCATCACATGCAAGTGCCAGCTGAGCGTGGGCTACATGCAC
TGGTATCAGCAGAAGCCTGGCAAGGCCCCTAAGCTGCTGATCTACGACAC
AAGCAAGCTGGCCTCTGGCGTGCCCAGCAGATTTTCTGGCTCTGGCAGCG
GCACCGCCTTCACACTGACCATCTCTAGCCTGCAGCCTGACGACTTCGCC
ACCTACTACTGCTTTCAAGGCAGCGGCTACCCTTTCACCTTTGGCGGCGG
AACAAAGCTGGAAATCAAG
PALIVIZUMAB VH
(SEQ ID NO: 2)
CAAGTGACCCTGAGAGAGTCTGGCCCTGCTCTGGTCAAGCCCACACAGA
CCCTGACACTGACCTGCACCTTCAGCGGCTTTAGCCTGAGCACAAGCGGC
ATGAGCGTCGGCTGGATTAGACAGCCTCCTGGCAAAGCCCTGGAATGGCT
GGCCGACATTTGGTGGGACGACAAGAAGGACTACAACCCCAGCCTGAAGT
CCCGGCTGACCATCAGCAAAGACACCAGCGCCAATCAGGTGGTGCTGAAA
GTGACCAACATGGACCCTGCCGACACCGCCACCTACTACTGTGCCAGATC
CATGATCACCAACTGGTACTTCGACGTGTGGGGAGCCGGCACCACAGTGA
CAGTTTCTTCT
CR3022 VH
(SEQ ID NO: 3)
CAGATGCAGCTGGTGCAGAGCGGCACCGAAGTGAAGAAGCCTGGCGAG
AGCCTGAAGATCAGCTGCAAAGGCAGCGGCTACGGCTTCATCACCTACTG
GATCGGCTGGGTCCGACAGATGCCTGGCAAAGGCCTTGAGTGGATGGGCA
TCATCTACCCCGGCGACAGCGAGACAAGATACAGCCCTAGCTTCCAGGGC
CAAGTGACCATCAGCGCCGACAAGAGCATCAACACCGCCTACCTGCAGTG
GTCCAGCCTGAAGGCCTCTGACACCGCCATCTACTATTGTGCCGGCGGAA
GCGGCATCAGCACCCCTATGGATGTTTGGGGCCAGGGCACCACCGTGACC
GTT
CR3022 VL
(SEQ ID NO: 4)
GACATCCAGCTGACACAGAGCCCCGATAGCCTGGCCGTGTCTCTGGGAG
AAAGAGCCACCATCAACTGCAAGAGCAGCCAGAGCGTGCTGTACTCCAGC
ATCAACAAGAACTACCTGGCCTGGTATCAGCAGAAGCCCGGCCAGCCTCC
TAAGCTGCTGATCTACTGGGCCAGCACCAGAGAAAGCGGCGTGCCCGATA
GATTTTCTGGCAGCGGCTCTGGCACCGACTTCACCCTGACAATTAGCTCC
CTGCAGGCCGAGGATGTGGCCGTGTACTACTGCCAGCAGTACTACAGCAC
CCCTTACACCTTTGGCCAGGGCACCAAGGTGGAAATC
B38 VH
(SEQ ID NO: 5)
GAGGTGCAGCTGGTTGAATCTGGCGGAGGACTGGTTCAGCCTGGCGGAT
CTCTGAGACTGTCTTGTGCCGCCAGCGGCTTCATCGTGTCCAGCAACTAC
ATGAGCTGGGTCCGACAGGCCCCTGGCAAAGGACTTGAATGGGTGTCCGT
GATCTACAGCGGCGGCAGCACCTACTACGCCGATTCTGTGAAGGGCAGAT
TCACCATCAGCCGGCACAACAGCAAGAACACCCTGTACCTGCAGATGAAC
AGCCTGAGAGCCGAGGACACCGCCGTGTACTACTGTGCCAGAGAAGCCTA
CGGCATGGACGTGTGGGGACAGGGAACAACCGTGACCGTTAGCTCT
B38 VL
(SEQ ID NO: 6)
GACATCGTGATGACTCAGAGCCCCAGCTTTCTGAGCGCCAGCGTGGGAG
ACAGAGTGACCATCACATGTAGAGCCAGCCAGGGCATCAGCAGCTACCTG
GCTTGGTATCAGCAGAAGCCTGGCAAGGCCCCTAAACTGCTGATCTACGC
CGCCAGCACACTGCAGTCTGGCGTGCCATCTAGATTTTCCGGCAGCGGCT
CTGGCACCGAGTTCACCCTGACCATATCTAGCCTGCAGCCTGAGGACTTC
GCCACCTACTACTGCCAGCAGCTGAACAGCTACCCTCCTTACACCTTTGG
CCAGGGCACCAAGCTGGAAATCAAG
2A12 VH
(SEQ ID NO: 7)
CAGGTGCAGCTGGTGCAGTCTGGGGCTGAAGTGAAGAAGCCTGGGGCCT
CAGTGAAGGTTTCCTGCAAGGCTTCTGGATACACCTTCACCGGCTACTAT
ATGCACTGGGTGCGACAGGCCCCTGGACAAGGGCTTGAGTGGATGGGACG
GATCAACCCTAACAGTGGTGGCACAAACTATGCACAGAAGTTTCAGGGCA
GGGTCACCATGACCAGGGACACGTCCATCAGCACAGCCTACATGGAGCTG
AGCAGGCTGAGATCTGACGACACGGCCGTGTATTACTGTGCGAGAGAAAT
GATTACTCTAGTTCAGGGAGTAAACTACTACTACATGGACGTCTGGGGCA
AAGGGACCACGGTCACCGTCTCCTCA
2A12 VL
(SEQ ID NO: 8)
CAGTCTGCCCTGACTCAGCCTGCCTCCGTGTCTGGGTCACCTGGACAGTC
GATCACCATCTCCTGCACTGGAACCAGCAGTGACGTTGGTGGTTATAACT
ATGTCTCCTGGTACCAACAGCACCCAGGCAAAGCCCCCAAACTCATGATT
TATGATGTCAGTAATCGGCCCTCAGGGGTTTCTAATCGCTTCTCTGGCTC
CAAGTCTGGCAACACGGCCTCCCTGACCATCTCTGGGCTCCAGGCTGAGG
ACGAGGCTGATTATTACTGCAGCTCATATACAAGCAGCAGCACTCTCGTG
GTATTCGGCGGAGGGACCAAGCTGACCGTCCTA
HC19 VH
(SEQ ID NO: 9)
CAGGTGCAGCTGAAAGAGTCTGGACCTGGACTGGTGGCCCCTAGCCAGA
GCCTGTCTATCACCTGTACCGTGTCCGGCTTCCTGCTGATCTCCAATGGC
GTGCACTGGGTCCGACAGCCTCCAGGCAAAGGACTGGAATGGCTGGGAGT
GATTTGGGCTGGCGGCAACACCAACTACAACAGCGCCCTGATGAGCCGGG
TGTCCATCAGCAAGGACAACAGCAAGAGCCAGGTGTTCCTGAAGATGAAG
TCCCTGCAGACCGACGACACCGCCATGTACTACTGCGCCAGAGACTTCTA
CGACTACGACGTGTTCTACTACGCCATGGATTACTGGGGCCAGGGCACCA
GCGTGACAGTCTCTTCT
HC19 VL
(SEQ ID NO: 10)
CAGGCCGTGGTCACACAAGAAAGCGCCCTGACAACAAGCCCTGGCGAGA
CAGTGACCCTGACCTGCAGATCTTCTACAGGCGCCGTGACCACCAGCAAC
TACGCCAATTGGGTGCAAGAGAAGCCCGACCACCTGTTCACAGGACTGAT
CGGCGGCACCAACAATAGAGCACCTGGCGTGCCAGCCAGATTCAGCGGAT
CTCTGATCGGAGACAAGGCCGCACTGACAATCACAGGCGCCCAGACAGAG
GACGAGGCCATCTACTTTTGCGCCCTGTGGTACAGCAACCACTGGGTTTT
CGGCGGAGGCACCAAGCTGACAGTTCTG
HC45 VH
(SEQ ID NO: 11)
CAGGTTCAGCTGCAACAGCCTGGCGCCGAACTTGTTAGACCTGGCGCCTC
TGTGAAGCTGAGCTGTAAAGCCAGCGGCTACACCCTGACCACCTACTGGA
TGAACTGGTTCAAGCAGCGGCCCGATCAGGGCCTCGAGTGGATCGGAAGA
ATCGACCCCTACGACAGCGAGACACACTACAACCAGAAGTTCAAGGACAA
GGCCATCCTGACCGTGGACAGAAGCAGCAGCACAGCCTACATGCAGCTGA
GCAGCCTGACCAGCGAAGATAGCGCCGTGTACTACTGCACCCGGTTTCTG
CAGATCACCACCATCATCTACGGCATGGACTACTGGGGCCAGGGCACAAG
CGTGACAGTCTCTTCT
HC45 VL
(SEQ ID NO: 12)
GACGTGGTCATGACACAGACCCCACTGAGCCTGCCTGTGTCTCTGGGAG
ATCAGGCCAGCATCAGCTGCAGATCCAGCCAGACACTGGTGCACAGCAAC
GGCAACACCTACCTGCACTGGTATCTGCAGAAGCCCGGACAGAGCCCCAA
GCTGCTGATCTACAAGGTGTCCAACCGGTTCAGCGGCGTGCCCGATAGAT
TTTCTGGCAGCGGCTCTGGCACCGACTTCACCCTGAAGATCTCCAGAGTG
GAAGCCGAGGACCTGGGCGTGTACTTCTGCAGCCAGAATACCCACGTGCC
ATACACCTTTGGCGGAGGCACCAAGCTGGAAATC
CR9114 VH
(SEQ ID NO: 13)
CAGGTTCAGCTGGTTCAGTCTGGCGCCGAAGTGAAGAAACCTGGCAGCA
GCGTGAAGGTGTCCTGCAAGTCTAGCGGCGGCACCAGCAACAACTACGCC
ATCTCTTGGGTCCGACAGGCCCCTGGACAAGGCTTGGATTGGATGGGCGG
CATCAGCCCTATCTTCGGCAGCACAGCCTACGCTCAGAAATTCCAGGGCA
GAGTGACCATCAGCGCCGACATCTTCAGCAACACCGCCTACATGGAACTG
AACAGCCTGACCAGCGAGGATACCGCCGTGTACTTCTGTGCCAGACACGG
CAATTACTACTACTACAGCGGCATGGACGTGTGGGGCCAGGGAACAACAG
TGACCGTTAGCTCT
CR9114 VL
(SEQ ID NO: 14)
CAGTCTGCTCTGACACAGCCTCCAGCCGTGTCTGGAACACCTGGCCAGA
GAGTGACCATCAGCTGTAGCGGCAGCGACAGCAATATCGGAAGGCGGAGC
GTGAACTGGTATCAGCAGTTCCCTGGCACAGCCCCTAAGCTGCTGATCTA
CAGCAACGACCAGCGGCCTAGCGTGGTGCCCGATAGATTTTCTGGCAGCA
AGAGCGGCACAAGCGCCAGCCTGGCTATTTCTGGACTGCAGAGCGAGGAC
GAGGCCGAGTATTATTGTGCCGCCTGGGACGATTCTCTGAAGGGCGCTGT
TTTTGGCGGCGGAACCCAGCTGACAGTTCTG
4A8 VH
(SEQ ID NO: 15)
AGCGAAGTGCAGCTGGTGGAGTCTGGGGCTGAGGTGAAGAAGCCTGGG
GCCTCAGTGAAGGTTTCCTGCAAGGTTTCCGGATACACCCTCACTGAATT
ATCCATGCACTGGGTGCGACAGGCTCCTGGAAAAGGGCTTGAGTGGATGG
GAGGTTTTGATCCTGAAGATGGTGAAACAATGTACGCACAGAAGTTCCAG
GGCAGAGTCACCATGACCGAGGACACATCTACAGACACAGCCTACATGGA
GCTGAGCAGCCTGAGATCTGAGGACACGGCCGTGTATTACTGTGCAACCT
CGACAGCAGTGGCTGGCACACCTGACCTCTTCGACTACTACTACGGTATG
GACGTCTGGGGCCAAGGGACCACGGTCACCGTCTCCTCAGCC
4A8 VL
(SEQ ID NO: 16)
TCCGAAATAGTGATGACGCAGTCTCCACTCTCCTCACCTGTCACCCTTGG
ACAGCCGGCCTCCATCTCCTGCAGGTCTAGTCAAAGCCTCGTACACAGTG
ATGGAAACACCTACTTGAGTTGGCTTCAGCAGAGGCCAGGCCAGCCTCCA
AGACTCCTAATTTATAAGATTTCTAACCGGTTCTCTGGGGTCCCAGACAG
ATTCAGTGGCAGTGGGGCAGGGACAGATTTCACACTGAAAATCAGCAGGG
TGGAAGCTGAGGATGTCGGGGTTTATTACTGCACGCAAGCTACACAATTT
CCGTACACTTTTGGCCAGGGGACCAAAGTGGATATCAAG
12A2 VH
(SEQ ID NO: 17)
CAGGTGCAGCTGGTGCAGTCTGGGGCTGAAGTGAAGAAGCCTGGGGCCT
CAGTGAAGGTTTCCTGCAAGGCTTCTGGATACACCTTCACCGGCTACTAT
ATGCACTGGGTGCGACAGGCCCCTGGACAAGGGCTTGAGTGGATGGGACG
GATCAACCCTAACAGTGGTGGCACAAACTATGCACAGAAGTTTCAGGGCA
GGGTCACCATGACCAGGGACACGTCCATCAGCACAGCCTACATGGAGCTG
AGCAGGCTGAGATCTGACGACACGGCCGTGTATTACTGTGCGAGAGAAAT
GATTACTCTAGTTCAGGGAGTAAACTACTACTACATGGACGTCTGGGGCA
AAGGGACCACGGTCACCGTCTCCTCA
12A2 VL
(SEQ ID NO: 18)
CAGTCTGCCCTGACTCAGCCTGCCTCCGTGTCTGGGTCACCTGGACAGTC
GATCACCATCTCCTGCACTGGAACCAGCAGTGACGTTGGTGGTTATAACT
ATGTCTCCTGGTACCAACAGCACCCAGGCAAAGCCCCCAAACTCATGATT
TATGATGTCAGTAATCGGCCCTCAGGGGTTTCTAATCGCTTCTCTGGCTC
CAAGTCTGGCAACACGGCCTCCCTGACCATCTCTGGGCTCCAGGCTGAGG
ACGAGGCTGATTATTACTGCAGCTCATATACAAGCAGCAGCACTCTCGTG
GTATTCGGCGGAGGGACCAAGCTGACCGTCCTA

### EXAMPLES

### Example 1: Functional Characterization of the REFORM Antibody Panel

To assess whether the Fc mutations affected the Fab domain, binding of each of the VIC16 REFORM antibodies (REFORMabs) to EBOV GP by ELISA (Table 5) was examined.

TABLE 5
Fab-associated Features of REFORM Library. Each REFORM
antibody variant was evaluated for binding to Ebola GP
by ELISA (Log10 area under the curve). “+”
in the binding column indicates positive binding; “−”
indicates binding was reduced by 50% compared to mIgG1
VIC16 and excluded from downstream analysis.

| | ELISA | |
|---|---|---|
| REFORM Variant | Log10 AUC | Binding |
| mIgG1 VIC16 | 0.831 | + |
| IgG1 | 0.661 | + |
| LALA | 0.709 | + |
| LALALS | 0.607 | + |
| LALAPG | 0.695 | + |
| N297Q | 0.581 | + |
| N297QLS | 0.484 | + |
| SAEAKA | 0.606 | + |
| SAEAKALS | 0.605 | + |
| E345R | 0.805 | + |
| E345RLS | 0.639 | + |
| HFST | 0.579 | + |
| HFSTLS | 0.662 | + |
| S324T | 0.632 | + |
| S324TLS | 0.429 | − |
| RGY | 0.67 | + |
| RGYLS | 0.592 | + |
| SEHFST | 0.598 | + |
| SEHFSTLS | 0.538 | + |
| KWES | 0.718 | + |
| KWESLS | 0.584 | + |
| E333A | 0.672 | + |
| I332E | 0.644 | + |
| I332ELS | 0.555 | + |
| IEGA | 0.721 | + |
| IEGALS | 0.533 | + |
| SDIE | 0.694 | + |
| SDIELS | 0.561 | + |
| SDIEAL | 0.735 | + |
| SDIEALLS | 0.552 | + |
| SDIEALYTE | 0.517 | + |
| SDIEALYTELS | 0.427 | − |
| SDIEGA | 0.713 | + |
| SDIEGALS | 0.622 | + |
| SDIESA | 0.685 | + |
| SDIESALS | 0.59 | + |
| SDIEALGA | 0.616 | + |
| SDIEALGALS | 0.539 | + |
| K326W | 0.648 | + |
| EFTEA | 0.718 | + |
| EFTEALS | 0.523 | + |
| AAA | 0.443 | − |
| EANA | 0.554 | + |
| N434W | 0.686 | + |
| MLNS | 0.605 | + |
| YTE | 0.638 | + |
| YTELS | 0.466 | − |
| DVNH | 0.667 | + |
| N434A | 0.501 | + |
| PINH | 0.187 | − |
| PIQI | 0.676 | + |
| PIQILS | 0.449 | − |
| QL | 0.787 | + |
| AALS | 0.505 | + |
| P257ILS | 0.219 | − |
| QLS | 0.595 | + |
| ALS | 0.438 | − |
| SELF | 0.7 | + |
| SELFLS | 0.597 | + |
| D376VLS | 0.531 | + |
| E380ALS | 0.496 | + |

Although the original VIC16 mAb was a murine IgG1 antibody, chimerization of VIC16 with human IgG Fc domains was previously shown not to affect its ability to bind to EBOV GP antigen (Zeitlin et al., 2011). Similarly, nearly all of the REFORMabs exhibited EBOV GP binding that was comparable to wild type. However, among the panel of 61 mAbs, 8 had significantly decreased (<50%) GP binding relative to the other mAbs in the panel, despite no apparent issues during production of these mAbs. Thus, 53 Fc-variants had binding capacities equivalent to the original IgG1 construct and could be used to examine the role of Fc-effector activity in in vivo protection.

To define the unique functional profiles across the VIC16 REFORMab panel, the ability of each Fc-variant to drive phagocytosis of GP-coated beads by monocytes (ADCP) and neutrophils (ADNP), as well as complement deposition (ADCD) onto GP-coated beads was profiled, as was the ability to induce NK cell degranulation (ADNKA: CD107a) and NK cell secretion of IFNγ (ADNKA: IFNγ) and MIP-1β (ADNKA: MIP-1β (FIG. 5A). All REFORMabs were compared to the original murine IgG1 VIC16 mAb. Interestingly, wild type VIC16-human IgG1 induce only ADCP activity. In contrast, a range of functional activity was clearly observed across the REFORMab panel (FIG. 5A). In particular, REFORMabs carrying Fc-variants shown to enhance ADCC activity, such as SDIE (Lazar et al., 2006), showed significantly elevated NK cell activation compared to the original VIC16 and the humanized IgG1 variant. Similarly, increased ADCP activity and ADCD activity were observed for variants with corresponding mutations that are predicted to enhance these functions. Although activation of neutrophils has not been routinely screened across Fc-variants, several mutants, including SDIE, exhibited enhanced ADNP and binding to FcγR3A (Lazar et al., 2006), highlighting additional effector functions that are present in this panel of Fc-variants.

Given that Fc is known to influence Fab activity (Kunert et al., 2004), the neutralizing activity of each VIC16 REFORMab was screened using vesicular stomatitis virus (VSV) pseudotyped to display EBOV GP. The neutralizing activity of each REFORMab was compared to the original murine IgG1 VIC16 mAb and the antibody KZ52 that shows strong neutralization activity and was used as a positive control (Parren et al., 2002).The ICso of the original VIC16 and KZ52 was 0.34 μg/ml and 0.08 μg/ml, respectively, which is consistent with previously published IC₅₀ values for both mAbs (Parren et al., 2002; Saphire et al., 2018a). The majority of REFORM antibodies showed potent neutralizing activity comparable to the wildtype VIC16 activity (IC₅₀<1 μg/ml), yet 8 mAbs lost neutralization activity while maintaining EBOV GP binding and induction of effector function (FIG. 5B). Of these, 7 of 8 (87.5%) were combinations of functional variants paired with the half-life extension mutation, LS (Zalevsky et al., 2010). An additional 20 REFORMabs had an IC₅₀>1 μg/ml and were considered to have lost neutralization potency compared to the rest of the panel. Interestingly, many of these antibodies still induced Fc-effector functions, albeit to a reduced level in some instances, indicating that specific combinations of changes in the Fc domain impacted Fab-mediated neutralizing activity as has been previously observed for engineered HIV-specific mAbs (Kunert et al., 2004).

For an overview of the polyfunctional profile of individual antibodies, for each REFORMab composite flower plots were generated wherein each effector function was represented by a color-coded petal that vary in size according to the relative activity of each function (FIG. 5C). Robust variation across all the Fc-variants having varying degrees of neutralization (dark blue petal) and addition of individual or combinations of Fc-effector functions was observed. Thus, this panel of REFORMabs provides a heterogeneous source of Fc-variation on a single antibody specificity, providing an opportunity to define specific protective correlates of immunity against EBOV.

### Example 2. Down-selection of REFORM Antibodies for In Vivo Evaluation

Among the 30 REFORMabs that retained neutralizing activity of IC₅₀<1 μg/ml we observed a wide breadth of profiles (FIG. 6A top). To define classes of functional variants, K-means clustering was used to identify unique functional clusters (FIG. 6A bottom). Across these 30 REFORMabs, 6 different groups emerged that captured distinct functional profiles including non-functional mAbs (cluster 1), predominantly monofunctional mAbs (clusters 2, 3), and polyfunctional antibodies (clusters 4-6). The monofunctional antibodies in cluster 2 induced only monocyte phagocytosis (ADCP). The sole mAb in cluster 3, KWES, exhibited high levels of complement deposition. Within the polyfunctional clusters, clusters 4 and 5 differed mainly by the magnitude of NK cell-mediated activity, with mAbs in clusters 4 and 5 exhibiting moderate and high levels of NK cell activation, respectively. Cluster 6 contained only one mAb, EFTEA, that induced all functions tested, including high levels of complement activity, yet moderate levels of NK cell activation.

Comparing the functional profiles of the different clusters of VIC16 REFORMabs to the profiles induced in EVD survivors (FIGS. 2A-2C), it was determined that EVD survivor cluster 5, which exhibited moderate levels of NK cell activation, but high complement and phagocytic activity, was similar to REFORM cluster 6 (EFTEA). Higher levels of complement activity in EVD cluster 4 were mimicked by REFORM cluster 3 (KWES), and the high levels of NK cell activation and phagocytic activity yet low complement activation in EVD cluster 2 were represented in REFORM cluster 5. The non-functional EVD cluster 9 was represented by REFORM cluster 1. Thus, the functional diversity identified in EVD survivors was mimicked by the functional diversity in the REFORM panel, and five REFORMabs were selected for in vivo analysis.

### Example 3: Distinct Functional Profiles Track With In Vivo Antibody-Mediated Protection

To define the specific Fc-effector profiles linked to protection, five representative REFORMabs representing the different functional profiles were produced. Binding affinity for Ebola GP, induction of each effector function, and neutralization and was re-measured for the large-scale batches of these five antibodies (FIG. 6B). IgG1 was monofunctional, and exhibited only monocyte phagocytosis; KWES induced high levels of complement deposition and moderate levels of ADCP and NK cell activation; LALA represented a non-functional Fc domain; SDIEALGA captured polyfunctional Fc domains with high NK cell activation and neutrophil phagocytosis; and EFTEA captured all effector functions with high levels of complement deposition, ADCP, and ADNP but moderate levels of NK cell activation.

The binding affinity of each mAb for both human and mouse FcγRs was measured by surface plasmon resonance (FIG. 6D; Table 6).

TABLE 6
FcR binding affinities of downselected VIC16 REFORMabs. The K_{D} (μM) values for the
indicated antibodies are shown and the fold-change relative to IgG1 is listed in brackets.

| Variant | huFcγR3A | | huFcγR2A | | huFcγR2B | | huFcγR3B | | mFcγRI | | mFcγRIV | | mFcγR3 | | mFcγR2b | |
|---|---|---|---|---|---|---|---|---|---|---|---|---|---|---|---|---|
| IgG1 | 6.1 | [1] | 30 | [1] | 23 | [1] | 25 | [1] | 0.08 | [1] | 0.24 | [1] | 2.83 | [1] | 14.9 | [1] |
| LALA | 1700 | [0.003] | 892 | [0.03] | ND | | ND | | ND | | ND | | 820.3 | [0.04] | ND | |
| SDIEALGA | 0.675 | [9] | 1.6 | [19] | 21 | [1.1] | 3.2 | [7.8] | 0.1 | [0.8] | 0.001 | [235] | 0.01 | [0.16] | 0.13 | [114] |
| EFTEA | 6.1 | [1] | 0.252 | [119] | 1.2 | [19] | 475 | [0.05] | 0.06 | [1.3] | 0.12 | [2] | 1.1 | [9] | 0.49 | [30] |

| | | | | | | | | | | | | | | | |
|---|---|---|---|---|---|---|---|---|---|---|---|---|---|---|---|
| KWES | 1.6 | [3.8] | 8.6 | [3.4] | 224 | [0.12] | ND | 0.07 | [1.1] | 0.16 | [1.5] | 0.02 | [0.94] | 46 | [0.3] |

ND = not detected

As expected, the SDIEALGA variant known to increase antibody affinity for the activating receptors FcγR3A and FcγR2A (Smith et al., 2012) exhibited a 9-fold and 18-fold increase in K_{D} for FcγR3A and FcγR2A, respectively, compared to the VIC16 human IgG1 (Table 6).

The K_{D} of the EFTEA variant for FcγR2A and the inhibitory FcγR2B was 120-fold and 19-fold higher compared to IgG1, respectively, consistent with increases described in previous reports (Moore et al., 2010). Only the SDIEALGA variant showed elevated binding to FcγR3B, a neutrophil-specific FcγR (Bruhns, 2012). The LALA variant showed significantly abrogated binding to the human FcγRs, as expected (Hessell et al., 2007). KWES demonstrated modest enhancement in binding to FcγR3A and FcγR2A, consistent with the moderate levels of NK cell activation and phagocytic activities observed, and no binding to FcγR3B was detected, which likely explains the limited ADNP activity seen for this variant.

Mounting evidence points to significant parallels in human IgG1:humanFcγR (hFcγR) and human IgG1:mouse FcγRs (mFcγR) binding profiles (Dekkers et al., 2017; Overdijk et al., 2012), with the exception of mFcγRI. However, whether the REFORMab hFcγR binding profiles translated across species was uncertain, although this feature is key to in vivo efficacy testing in mice. Similar to differences in hFcγR binding profiles, the SDIEALGA variant (14-fold increase) exhibited enhanced binding to mFcγRIV (FIG. 6D; Table 5), which in mice is expressed on monocytes/macrophages and neutrophils and is associated with ADCC (Nimmerjahn et al., 2005). EFTEA showed increased affinity for mFcγRIII (9-fold increase), present on all murine myeloid cells and the sole mFcγR expressed on resting murine NK cells (Bruhns, 2012), whereas SDIEALGA exhibited a 6-fold decrease in binding to mFcγRIII compared to IgG1, thus highlighting differential capacities of the variants to engage resting murine NK cells. The SDIEALGA and EFTEA variants showed a modest (4-fold and 2-fold, respectively) increase in binding to mFcγRIIB, the inhibitory receptor present on murine myeloid cells and B cells. Although the KWES variant exhibited an enhanced ability to activate complement, a pathway that is highly conserved across species (Nonaka and Kimura, 2006), its binding to other mFcγRs did not increase. The LALA variant, which showed the expected abrogation of binding to human FcγRs, exhibited abolished binding to mFcγRI, mFcγRIV and mFcγRIIb, and weak binding to mFcγRIII. All other antibodies (IgG1, EFTEA, SDIEALGA, and KWES) demonstrated comparable binding to the high affinity mFcγRI, present on murine dendritic cells. As immune complexes increase avidity of antibodies to low-affinity FcγRs, analysis of murine FcγRIIb and FcγRIII binding to Ebola GP:mAb immune complexes was performed. The low affinity murine FcγR bound to all of the immune complexes with the exception of the LALA variant immune complexes (FIG. 6E). The mFcγRIIb bound highest to the SDIEALGA variant immune complex, consistent with the high binding affinity observed by SPR, whereas KWES showed elevated binding to mFcγRIII compared with the other mAbs (FIG. 6E).

To begin to start developing comparative rules sets to translate binding between human and mouse FcRs, we determined the FcγR binding affinities across a subset of the VIC16 REFORM panel to both human and mouse FcγRs (FIG. 7A). Strong concordance in binding affinity was observed between mFcγRIV and human huFγR3A and huFcγR3B, indicating similarity between these receptors and highlighting that Fc mutations aimed at enhancing binding to huFcγR3A will likely also enhance binding to mFcγRIV. Accordingly, affinity to mFcγRIV was strongly associated with human NK cell and neutrophil effector functions (FIG. 7B). Moderate concordance was observed between mFcγRIV and huFcγR2A high-affinity variant H131, highlighting parallels in across the non-FcγRI high affinity activating FcγRs between species. Together, these data highlight the unique binding potential of the REFORMabs to both human and mouse FcRs and provide the foundation to build a comparative rule set to translate FcR-meditated activities across species that will help provide critical insights for potential in vivo differential therapeutic benefit.

### Example 4: Dissecting Fc-Correlates of Immunity In Vivo

To define Fc-correlates that contribute to antiviral immunity in vivo, the performance of REFORMabs was tested in a stringent murine model of Ebola virus infection (Bornholdt et al., 2016; Bray et al., 1998; Saphire et al., 2018a; Wec et al., 2017; Zeitlin et al., 2011). For each REFORMab, groups of 5 BALB/c mice were infected with 1,000 plaque-forming units (pfu) of mouse-adapted Ebola virus, 1 day before administration of 100 μg of antibody per mouse. Mice were monitored for survival, clinical disease score, and weight loss for 28 days (FIGS. 8A-8B). As expected, PBS-treated mice succumbed to infection by day 5 post-infection, developing clinical disease and weight loss by day 2 post-infection (FIG. 8A), highlighting the very aggressive and acute nature of the infection. The LALA variant, which showed loss of hFcγR binding but retained weak binding to mFcγRIII, protected 80% (4/5) of the mice, suggesting that neutralization activity with some mFcγR recruitment was sufficient to protect a majority of animals. However, animals given LALA REFORMabs lost weight and developed disease signs through day 12 (FIG. 8B). The animals ultimately survived infection, but the treatment with these REFORMAbs could not prevent disease. Similarly, wild type IgG1, which induced a monofunctional monocyte phagocytic response, also protected 80% of animals, but did not prevent development of disease signs or weight loss. In contrast, the polyfunctional variant SDIEALGA that exhibited robust NK cell activation, but lacked complement activation, protected only 60% of the mice from death, and did not prevent development of disease. The EFTEA and KWES variants conferred 100% survival in treated animals, and, remarkably, the animals showed no evidence of development of disease. Only one animal treated with the EFTEA variant showed mild clinical signs at day 5 post-infection (FIG. 8A). Both EFTEA and KWES induced high levels of complement activation and exhibited polyfunctional activity, with moderate induction of NK cell-mediated activity and monocyte phagocytosis.

To further confirm the protective effects of EFTEA and KWES, a second group of mice were dosed with approximately one-third (30 μg/mouse) of the original antibody dose (FIGS. 8C-8D). Animals treated with wild type, monofunctional IgG1 at this reduced dose all succumbed to Ebola virus infection. In contrast, animals treated with reduced doses of either EFTEA and KWES variants rapidly recovered from symptoms and disease and all survived (FIGS. 8C-8D). These data suggest that even at low doses of antibodies carrying these modifications, animals could recover and survive a lethal viral challenge, which points to the critical importance of complement and low-level ADCC as mechanistic correlates of protection.

### Example 5: Immune-Protective Versus Immunopathological Role of Antibodies is Compared in a Panel of Monoclonals to SARS-CoV-2

While the development of vaccines against SARS-CoV-2 are underway, therapeutics, are urgently needed to support those with more severe infection. Among the fastest moving classes of therapeutics, monoclonal antibodies have begun to show some promise. However, data from SARS-CoV immunized non-human primates pointed to the potential role of neutralizing antibodies in enhancing disease, via the induction of inflammatory responses, suggesting that caution is warranted in the application of monoclonal antibody therapeutics for SARS-CoV-2 treatment.

To specifically explore the role of protection versus enhancement, an Fc-engineering system was developed to rapidly graft 80 distinct Fc-domains- each with a different Fc-functional property- onto emerging SARS-CoV-2 specific monoclonal antibodies. The system has been used to develop libraries of Fc-engineered neutralizing and non-neutralizing antibodies across the Spike (S) antigen. Preliminary data highlight the role of a disease enhancing Fc-effector functional non-neutralizing cross-SARS-reactive antibody, CR3022, tested in both mice and hamsters. The data provide the first model to begin to dissect enhancement in SARS-CoV-2 to advance therapeutic and vaccine development. Additional libraries have been constructed on emerging neutralizing SARS-CoV-2 antibodies.

To explore the potential immune-protective versus-immunopathological role of antibodies, a panel of monoclonals to SARS-CoV-2 was engineered: a receptor binding domain (RBD)-specific non-neutralizer (CR3022), an RBD-specific neutralizer (B38), and a non-RBD-specific N-terminal domain (NTD)-specific neutralizer (A12). These three antibodies offered a unique opportunity to probe the overall landscape of “conventional” (most representative of the current portfolio of monoclonals) therapeutics on the market. The antibodies were engineered to have 1 of 80 different Fc-domains. The antibodies were all functionally profiled prior to dowselection for animal testing.

The CR3022 variant was taken through a full panel of testing. All variants were generated, all variants were profiled (FIG. 10), and both therapeutic mouse and hamster experiments were performed. The results of the CR3022 studies demonstrate the first evidence of authentic disease enhancement in both mice and hamsters using a non-neutralizing Fc-modified antibody.

Specifically, using 3 down-selected Fc-variants (a wildtype IgG1, a Fc-knockout (KO), and an Fc-enhanced variant), BALB/c mice were treated 12 hours following SARS-CoV-2 infection to probe for therapeutic protective Fc-functions. BALB/c mice were infected with 10⁵ pfu of mouse adapted SARS-CoV-2 I.N, treated with 200 ug of the WT, Fc-knockout (Fc-KO) or control antibody or 100 ug of the Fc-enhanced antibody I.P., and monitored for 2 days for lung viral titer and weight loss, with five mice per group (FIG. 11). Slightly higher, but non-significant, differences were observed in viral replication using the wildtype CR3022 IgG1 compared to the control antibody (FIG. 11). In contrast, significantly lower levels of viral replication were observed in mice treated with the Fc-KO CR3022 variant compared to the wildtype antibody. Moreover, reduction in viral load was observed with the Fc-enhanced CR3022 variant (FIG. 11). However, significant weight loss occurred in both wildtype and Fc-enhanced variant treated mice and minimal to no weight loss in mice treated with the Fc-KO variants. Thus, while the wildtype CR3022 IgG1 and the Fc-enhanced CR3022 variants showed divergent virologic effects, both variants led to enhanced pathology. The disconnect between viral load and weight-loss for the Fc-enhanced CR3022 antibody raised the possibility that the Fc-enhanced variant may have pathological consequences.

To further understand the role of Fc-enhanced pathology, the therapeutic benefit of the CR3022 variants was tested in a more pathological model of SARS-CoV-2 infection. Syrian golden hamsters are highly susceptible to SARS-CoV-2 infection and develop severe infection after challenge. Thus, using this model, the panel of Fc-engineered monoclonals was assessed. Hamsters were challenged intranasally with 10⁶ pfu/mL (10⁷ TCID50) of SARS-CoV-2, and 1 day post infection, treated with 5 mg/kg IgG1, Fc-enhanced, Fc-KO, or a control antibody, with 5 hamsters per group. Weight was monitored daily, and lung viral titers were determined 3 days post infection (FIG. 12). Similar to the results observed in the mouse model (FIG. 12), the wildtype CR3022 had no impact on viral load compared to control antibody treated animals (FIG. 11). In contrast to mice, hamsters did not experience any benefit from the Fc-KO antibody (FIG. 12) likely due to the more severe nature of the infection in this model. Conversely, hamsters treated with the Fc-enhanced CR3022 exhibited increased viral load in the lung and increased weight loss (FIG. 12). Despite the viral load disparity across the mice and hamsters, both models exhibited increased weight loss upon treatment with the Fc-enhanced CR3022 (FIGS. 11 and 12), suggesting similar host-responses to the Fc-enhanced monoclonal. Thus, collectively, these data point to the critical importance of balancing Fc-effector function to temper pathology in susceptible populations.

Using Fc-engineering the first signals of disease enhancement across animal models was observed. However, whether the same will be observed with neutralizing antibodies to different specificities remains unclear. Given the fears of antibody-dependent enhancement (ADE) that is looming over the vaccine and monoclonal development world, dissecting both the protective and pathological roles of antibodies is possible using this system.

The mechanistic value of engineering and defining the protective/pathological balance of antibodies will offer critical insights to advance therapeutic and vaccine design.

### REFERENCES

All patents, patent applications and publications mentioned in this specification are herein incorporated by reference to the same extent as if each independent patent and publication was specifically and individually indicated to be incorporated by reference.
1. Ackerman, M. E., Das, J., Pittala, S., Broge, T., Linde, C., Suscovich, T. J., Brown, E. P., Bradley, T., Natarajan, H., Lin, S., et al. (2018). Route of immunization defines multiple mechanisms of vaccine-mediated protection against SIV. Nature medicine.
2. Agnihothram, S., Gopal, R., Yount, B. L., Jr, Donaldson, E. F., Menachery, V. D., Graham, R. L., Scobey, T. D., Gralinski, L. E., Denison, M. R., Zambon, M., et al. (2013). Evaluation of Serologic and Antigenic Relationships Between Middle Eastern Respiratory Syndrome Coronavirus and Other Coronaviruses to Develop Vaccine Platforms for the Rapid Response to Emerging Coronaviruses. The Journal of infectious diseases 209, 995-1006.
3. Baseler, L., Chertow, D. S., Johnson, K. M., Feldmann, H., and Morens, D. M. (2017). The Pathogenesis of Ebola Virus Disease. Annual review of pathology 12, 387-418.
4. Bornholdt, Z. A., Herbert, A. S., Mire, C. E., He, S., Cross, R. W., Wec, A. Z., Abelson, D. M., Geisbert, J. B., James, R. M., Rahim, M. N., et al. (2019). A Two-Antibody Pan-Ebolavirus Cocktail Confers Broad Therapeutic Protection in Ferrets and Nonhuman Primates. Cell host & microbe 25, 49-58 e45.
5. Bornholdt, Z. A., Turner, H. L., Murin, C. D., Li, W., Sok, D., Souders, C. A., Piper, A. E., Goff, A., Shamblin, J. D., Wollen, S. E., et al. (2016). Isolation of potent neutralizing antibodies from a survivor of the 2014 Ebola virus outbreak. Science 351, 1078-1083.
6. Bournazos, S., DiLillo, D. J., Goff, A. J., Glass, P. J., and Ravetch, J. V. (2019). Differential requirements for FcgammaR engagement by protective antibodies against Ebola virus. Proceedings of the National Academy of Sciences of the United States of America 116, 20054-20062.
7. Bray, M., Davis, K., Geisbert, T., Schmaljohn, C., and Huggins, J. (1998). A mouse model for evaluation of prophylaxis and therapy of Ebola hemorrhagic fever. The Journal of infectious diseases 178, 651-661.
8. Bruhns, P. (2012). Properties of mouse and human IgG receptors and their contribution to disease models. Blood 119, 5640-5649.
9. Chu, S. Y., Vostiar, I., Karki, S., Moore, G. L., Lazar, G. A., Pong, E., Joyce, P. F., Szymkowski, D. E., and Desjarlais, J. R. (2008). Inhibition of B cell receptor-mediated activation of primary human B cells by coengagement of CD19 and FcgammaRIIb with Fc-engineered antibodies. Molecular immunology 45, 3926-3933.
10. Corti, D., Misasi, J., Mulangu, S., Stanley, D. A., Kanekiyo, M., Wollen, S., Ploquin, A., Doria-Rose, N. A., Staupe, R. P., Bailey, M., et al. (2016). Protective monotherapy against lethal Ebola virus infection by a potently neutralizing antibody. Science 351, 1339-1342.
11. Corti, D., Zhao, J., Pedotti, M., Simonelli, L., Agnihothram, S., Fett, C., Fernandez-Rodriguez, B., Foglierini, M., Agatic, G., Vanzetta, F., et al. (2015). Prophylactic and postexposure efficacy of a potent human monoclonal antibody against MERS coronavirus. Proceedings of the National Academy of Sciences of the United States of America 112, 10473-10478.
12. Dall'Acqua, W. F., Woods, R. M., Ward, E. S., Palaszynski, S. R., Patel, N. K., Brewah, Y. A., Wu, H., Kiener, P. A., and Langermann, S. (2002). Increasing the affinity of a human IgG1 for the neonatal Fc receptor: biological consequences. Journal of immunology 169, 5171-5180.
13. Datta-Mannan, A., Witcher, D. R., Tang, Y., Watkins, J., Jiang, W., and Wroblewski, V. J. (2007). Humanized IgG1 variants with differential binding properties to the neonatal Fc receptor: relationship to pharmacokinetics in mice and primates. Drug Metab Dispos 35, 86-94.
14. Dejnirattisai, W., Jumnainsong, A., Onsirisakul, N., Fitton, P., Vasanawathana, S., Limpitikul, W., Puttikhunt, C., Edwards, C., Duangchinda, T., Supasa, S., et al. (2010). Cross-reacting antibodies enhance dengue virus infection in humans. Science 328, 745-748.
15. Dekkers, G., Bentlage, A. E. H., Stegmann, T. C., Howie, H. L., Lissenberg-Thunnissen, S., Zimring, J., Rispens, T., and Vidarsson, G. (2017). Affinity of human IgG subclasses to mouse Fc gamma receptors. mAbs 9, 767-773.
16. Deming, D., Sheahan, T., Heise, M., Yount, B., Davis, N., Sims, A., Suthar, M., Harkema, J., Whitmore, A., Pickles, R., et al. (2006). Vaccine efficacy in senescent mice challenged with recombinant SARS-CoV bearing epidemic and zoonotic spike variants. PLoS medicine 3, e525.
17. Diebolder, C. A., Beurskens, F. J., de Jong, R. N., Koning, R. I., Strumane, K., Lindorfer, M. A., Voorhorst, M., Ugurlar, D., Rosati, S., Heck, A. J., et al. (2014). Complement is activated by IgG hexamers assembled at the cell surface. Science 343, 1260-1263.
18. DiLillo, D. J., Tan, G. S., Palese, P., and Ravetch, J. V. (2014). Broadly neutralizing hemagglutinin stalk-specific antibodies require FcgammaR interactions for protection against influenza virus in vivo. Nature medicine 20, 143-151.
19. Engler, C., Kandzia, R., and Marillonnet, S. (2008). A one pot, one step, precision cloning method with high throughput capability. PLoS One 3, e3647.
20. Fischinger, S., Fallon, J. K., Michell, A. R., Broge, T., Suscovich, T. J., Streeck, H., and Alter, G. (2019). A high-throughput, bead-based, antigen-specific assay to assess the ability of antibodies to induce complement activation. Journal of immunological methods 473, 112630.
21. Flyak, A. I., Shen, X., Murin, C. D., Turner, H. L., David, J. A., Fusco, M. L., Lampley, R., Kose, N., Ilinykh, P. A., Kuzmina, N., et al. (2016). Cross-Reactive and Potent Neutralizing Antibody Responses in Human Survivors of Natural Ebolavirus Infection. Cell.
22. Gunn, B. M., Roy, V., Karim, M. M., Hartnett, J. N., Suscovich, T. J., Goba, A., Momoh, M., Sandi, J. D., Kanneh, L., Andersen, K. G., et al. (2020). Survivors of Ebola Virus Disease Develop Polyfunctional Antibody Responses. The Journal of infectious diseases 221, 156-161.
23. Gunn, B. M., Yu, W. H., Karim, M. M., Brannan, J. M., Herbert, A. S., Wec, A. Z., Halfmann, P. J., Fusco, M. L., Schendel, S. L., Gangavarapu, K., et al. (2018). A Role for Fc Function in Therapeutic Monoclonal Antibody-Mediated Protection against Ebola Virus. Cell host & microbe 24, 221-233 e225.
24. He, W., Tan, G. S., Mullarkey, C. E., Lee, A. J., Lam, M. M., Krammer, F., Henry, C., Wilson, P. C., Ashkar, A. A., Palese, P., et al. (2016). Epitope specificity plays a critical role in regulating antibody-dependent cell-mediated cytotoxicity against influenza A virus. Proceedings of the National Academy of Sciences of the United States of America 113, 11931-11936.
25. Hessell, A. J., Hangartner, L., Hunter, M., Havenith, C. E., Beurskens, F. J., Bakker, J. M., Lanigan, C. M., Landucci, G., Forthal, D. N., Parren, P. W., et al. (2007). Fc receptor but not complement binding is important in antibody protection against HIV. Nature 449,101-104.
26. Hinton, P. R., Johlfs, M. G., Xiong, J. M., Hanestad, K., Ong, K. C., Bullock, C., Keller, S., Tang, M. T., Tso, J. Y., Vasquez, M., et al. (2004). Engineered human IgG antibodies with longer serum half-lives in primates. The Journal of biological chemistry 279, 6213-6216.
27. Idusogie, E. E., Presta, L. G., Gazzano-Santoro, H., Totpal, K., Wong, P. Y., Ultsch, M., Meng, Y. G., and Mulkerrin, M. G. (2000). Mapping of the C1q binding site on rituxan, a chimeric antibody with a human IgG1 Fc. Journal of immunology 164, 4178-4184.
28. Idusogie, E. E., Wong, P. Y., Presta, L. G., Gazzano-Santoro, H., Totpal, K., Ultsch, M., and Mulkerrin, M. G. (2001). Engineered antibodies with increased activity to recruit complement. Journal of immunology 166, 2571-2575.
29. Ilinykh, P. A., Graber, J., Kuzmina, N. A., Huang, K., Ksiazek, T. G., Crowe, J. E., Jr., and Bukreyev, A. (2018). Ebolavirus Chimerization for the Development of a Mouse Model for Screening of Bundibugyo-Specific Antibodies. J Infect Dis 218, S418-S422.
30. Jaume, M., Yip, M. S., Cheung, C. Y., Leung, H. L., Li, P. H., Kien, F., Dutry, I., Callendret, B., Escriou, N., Altmeyer, R., et al. (2011). Anti-Severe Acute Respiratory Syndrome Coronavirus Spike Antibodies Trigger Infection of Human Immune Cells via a pH- and Cysteine Protease-Independent FcγR Pathway. Journal of virology 85, 10582-10597.
31. Ju, B., Zhang, Q., Ge, X., Wang, R., Yu, J., Shan, S., Zhou, B., Song, S., Tang, X., Yu, J., et al. (2020). Potent human neutralizing antibodies elicited by SARS-CoV-2 infection. bioRxiv.
32. Kunert, R., Wolbank, S., Stiegler, G., Weik, R., and Katinger, H. (2004). Characterization of molecular features, antigen-binding, and in vitro properties of IgG and IgM variants of 4E10, an anti-HIV type 1 neutralizing monoclonal antibody. AIDS research and human retroviruses 20, 755-762.
33. Lazar, G. A., Dang, W., Karki, S., Vafa, O., Peng, J. S., Hyun, L., Chan, C., Chung, H. S., Eivazi, A., Yoder, S. C., et al. (2006). Engineered antibody Fc variants with enhanced effector function. Proceedings of the National Academy of Sciences of the United States of America 103, 4005-4010.
34. Liu, L., Wei, Q., Lin, Q., Fang, J., Wang, H., Kwok, H., Tang, H., Nishiura, K., Peng, J., Tan, Z., et al. (2019). Anti-spike IgG causes severe acute lung injury by skewing macrophage responses during acute SARS-CoV infection. JCI Insight 4.
35. Maruyama, T., Rodriguez, L. L., Jahrling, P. B., Sanchez, A., Khan, A. S., Nichol, S. T., Peters, C. J., Parren, P. W., and Burton, D. R. (1999). Ebola virus can be effectively neutralized by antibody produced in natural human infection. Journal of virology 73, 6024-6030.
36. Misasi, J., Gilman, M. S., Kanekiyo, M., Gui, M., Cagigi, A., Mulangu, S., Corti, D., Ledgerwood, J. E., Lanzavecchia, A., Cunningham, J., et al. (2016). Structural and molecular basis for Ebola virus neutralization by protective human antibodies. Science 351, 1343-1346.
37. Moldt, B., Schultz, N., Dunlop, D. C., Alpert, M. D., Harvey, J. D., Evans, D. T., Poignard, P., Hessell, A. J., and Burton, D. R. (2011). A panel of IgG1 b12 variants with selectively diminished or enhanced affinity for Fcgamma receptors to define the role of effector functions in protection against HIV. J Virol 85, 10572-10581.
38. Moore, G. L., Chen, H., Karki, S., and Lazar, G. A. (2010). Engineered Fc variant antibodies with enhanced ability to recruit complement and mediate effector functions. mAbs 2, 181-189.
39. Nimmerjahn, F., Bruhns, P., Horiuchi, K., and Ravetch, J. V. (2005). FcgammaRIV: a novel FcR with distinct IgG subclass specificity. Immunity 23, 41-51.
40. Nonaka, M., and Kimura, A. (2006). Genomic view of the evolution of the complement system. Immunogenetics 58, 701-713.
41. Olinger, G. G., Jr., Pettitt, J., Kim, D., Working, C., Bohorov, O., Bratcher, B., Hiatt, E., Hume, S. D., Johnson, A. K., Morton, J., et al. (2012). Delayed treatment of Ebola virus infection with plant-derived monoclonal antibodies provides protection in rhesus macaques. Proceedings of the National Academy of Sciences of the United States of America 109, 18030-18035.
42. Overdijk, M. B., Verploegen, S., Ortiz Buijsse, A., Vink, T., Leusen, J. H., Bleeker, W. K., and Parren, P. W. (2012). Crosstalk between human IgG isotypes and murine effector cells. Journal of immunology 189, 3430-3438.
43. Parren, P. W., Geisbert, T. W., Maruyama, T., Jahrling, P. B., and Burton, D. R. (2002). Pre- and postexposure prophylaxis of Ebola virus infection in an animal model by passive transfer of a neutralizing human antibody. Journal of virology 76, 6408-6412.
44. Pinto, D., Park, Y.-J., Beltramello, M., Walls, A. C., Tortorici, M. A., Bianchi, S., Jaconi, S., Culap, K., Zatta, F., De Marco, A., et al. (2020). Structural and functional analysis of a potent sarbecovirus neutralizing antibody. bioRxiv.
45. Polack, F. P., Teng, M. N., Collins, P. L., Prince, G. A., Exner, M., Regele, H., Lirman, D. D., Rabold, R., Hoffman, S. J., Karp, C. L., et al. (2002). A role for immune complexes in enhanced respiratory syncytial virus disease. The Journal of experimental medicine 196, 859-865.
46. Qiu, X., Wong, G., Audet, J., Bello, A., Fernando, L., Alimonti, J. B., Fausther-Bovendo, H., Wei, H., Aviles, J., Hiatt, E., et al. (2014). Reversion of advanced Ebola virus disease in nonhuman primates with ZMapp. Nature 514, 47-53.
47. Richards, J. O., Karki, S., Lazar, G. A., Chen, H., Dang, W., and Desjarlais, J. R. (2008). Optimization of antibody binding to FcgammaRIIa enhances macrophage phagocytosis of tumor cells. Mol Cancer Ther 7, 2517-2527.
48. Saphire, E. O., Schendel, S. L., Fusco, M. L., Gangavarapu, K., Gunn, B. M., Wec, A. Z., Halfmann, P. J., Brannan, J. M., Herbert, A. S., Qiu, X., et al. (2018a). Systematic Analysis of Monoclonal Antibodies against Ebola Virus GP Defines Features that Contribute to Protection. Cell 174, 938-952 e913.
49. Saphire, E. O., Schendel, S. L., Gunn, B. M., Milligan, J. C., and Alter, G. (2018b). Antibody-mediated protection against Ebola virus. Nature immunology 19, 1169-1178.
50. Shields, R. L., Namenuk, A. K., Hong, K., Meng, Y.G., Rae, J., Briggs, J., Xie, D., Lai, J., Stadlen, A., Li, B., et al. (2001). High resolution mapping of the binding site on human IgG1 for Fc gamma RI, Fc gamma RII, Fc gamma RIII, and FcRn and design of IgG1 variants with improved binding to the Fc gamma R. The Journal of biological chemistry 276, 6591-6604.
51. Smith, P., DiLillo, D. J., Bournazos, S., Li, F., and Ravetch, J. V. (2012). Mouse model recapitulating human Fcgamma receptor structural and functional diversity. Proceedings of the National Academy of Sciences of the United States of America 109, 6181-6186.
52. Stavenhagen, J. B., Gorlatov, S., Tuaillon, N., Rankin, C.T., Li, H., Burke, S., Huang, L., Vijh, S., Johnson, S., Bonvini, E., et al. (2007). Fc optimization of therapeutic antibodies enhances their ability to kill tumor cells in vitro and controls tumor expansion in vivo via low-affinity activating Fcgamma receptors. Cancer research 67, 8882-8890.
53. Steurer, W., Nickerson, P. W., Steele, A. W., Steiger, J., Zheng, X. X., and Strom, T. B. (1995). Ex vivo coating of islet cell allografts with murine CTLA4/Fc promotes graft tolerance. Journal of immunology 155, 1165-1174.
54. Sun, P., Williams, M., Nagabhushana, N., Jani, V., Defang, G., and Morrison, B. J. (2019). NK Cells Activated through Antibody-Dependent Cell Cytotoxicity and Armed with Degranulation/IFN-gamma Production Suppress Antibody-dependent Enhancement of Dengue Viral Infection. Scientific reports 9, 1109.
55. van Erp, E. A., Luytjes, W., Ferwerda, G., and van Kasteren, P. B. (2019). Fc-Mediated Antibody Effector Functions During Respiratory Syncytial Virus Infection and Disease. Frontiers in immunology 10, 548.
56. Walker, L. M., and Burton, D. R. (2018). Passive immunotherapy of viral infections: ‘super-antibodies’ enter the fray. Nature reviews Immunology 18, 297-308.
57. Wan, Y., Shang, J., Sun, S., Tai, W., Chen, J., Geng, Q., He, L., Chen, Y., Wu, J., Shi, Z., et al. (2020). Molecular Mechanism for Antibody-Dependent Enhancement of Coronavirus Entry. Journal of virology 94.
58. Wang, S. F., Tseng, S. P., Yen, C. H., Yang, J. Y., Tsao, C. H., Shen, C. W., Chen, K. H., Liu, F. T., Liu, W. T., Chen, Y. M., et al. (2014). Antibody-dependent SARS coronavirus infection is mediated by antibodies against spike proteins. Biochemical and biophysical research communications 451, 208-214.
59. Wang, T. T., Sewatanon, J., Memoli, M. J., Wrammert, J., Bournazos, S., Bhaumik, S. K., Pinsky, B. A., Chokephaibulkit, K., Onlamoon, N., Pattanapanyasat, K., et al. (2017). IgG antibodies to dengue enhanced for FcgammaRIIIA binding determine disease severity. Science 355, 395-398.
60. Warfield, K. L., Perkins, J. G., Swenson, D. L., Deal, E. M., Bosio, C. M., Aman, M. J., Yokoyama, W. M., Young, H. A., and Bavari, S. (2004). Role of natural killer cells in innate protection against lethal ebola virus infection. The Journal of experimental medicine 200, 169-179.
61. Wec, A. Z., Herbert, A. S., Murin, C. D., Nyakatura, E. K., Abelson, D. M., Fels, J. M., He, S., James, R. M., de La Vega, M. A., Zhu, W., et al. (2017). Antibodies from a Human Survivor Define Sites of Vulnerability for Broad Protection against Ebolaviruses. Cell 169, 878-890 e815.
62. Weiner, L. M., Dhodapkar, M. V., and Ferrone, S. (2009). Monoclonal antibodies for cancer immunotherapy. Lancet 373, 1033-1040.
63. Xu, D., Alegre, M. L., Varga, S. S., Rothermel, A. L., Collins, A. M., Pulito, V. L., Hanna, L. S., Dolan, K. P., Parren, P. W., Bluestone, J. A., et al. (2000). In vitro characterization of five humanized OKT3 effector function variant antibodies. Cell Immunol 200, 16-26.
64. Yeung, Y. A., Leabman, M. K., Marvin, J. S., Qiu, J., Adams, C. W., Lien, S., Starovasnik, M. A., and Lowman, H. B. (2009). Engineering human IgG1 affinity to human neonatal Fc receptor: impact of affinity improvement on pharmacokinetics in primates. Journal of immunology 182, 7663-7671.
65. Zalevsky, J., Chamberlain, A. K., Horton, H. M., Karki, S., Leung, L W., Sproule, T. J., Lazar, G. A., Roopenian, D. C., and Desjarlais, J. R. (2010). Enhanced antibody half-life improves in vivo activity. Nat Biotechnol 28, 157-159.
66. Zeitlin, L., Pettitt, J., Scully, C., Bohorova, N., Kim, D., Pauly, M., Hiatt, A., Ngo, L., Steinkellner, H., Whaley, K. J., et al. (2011). Enhanced potency of a fucose-free monoclonal antibody being developed as an Ebola virus immunoprotectant. Proceedings of the National Academy of Sciences of the United States of America 108, 20690-20694.
67. Zhao, X., Howell, K. A., He, S., Brannan, J. M., Wec, A. Z., Davidson, E., Turner, H. L., Chiang, C. I., Lei, L., Fels, J. M., et al. (2017). Immunization-Elicited Broadly Protective Antibody Reveals Ebolavirus Fusion Loop as a Site of Vulnerability. Cell 169, 891-904 e815.
68. Zhang, L., Leng, Q., Mixson, J. A., (2005). Alteration in the IL-2 signal peptide affects secretion of proteins in vitro and in vivo. J. Gene Med. 7, 354-65.

1. An antibody comprising an Fab binding domain that binds to the Ebola virus glycoprotein, and an Fc domain comprising constant heavy (CH)2 and CH3 domains,
wherein the Fab binding domain comprises
(a) a heavy chain variable region (VH) comprising a VH-complementarity determining region (CDR)1, a VH-CDR2, and a VH-CDR3 from the amino acid sequence of SEQ ID NO:25; and
(b) a light chain variable region (VL) comprising a VL-CDR1, a VL-CDR2, and a VL-CDR3 from the amino acid sequence of SEQ ID NO:26; and
wherein the Fc domain comprises an amino acid sequence with at least 95% sequence identity to the amino acid sequence set forth in any one of SEQ ID NOs: 35-95.

2. The antibody of claim 1, wherein the VH comprises the VH-CDR1 comprising the amino acid sequence of SEQ ID NO: 19, the VH-CDR2 comprising the amino acid sequence of SEQ ID NO: 20, and the VH-CDR3 comprising the amino acid sequence of SEQ ID NO: 21; and the VL comprises the VL-CDR1 comprising the amino acid sequence of SEQ ID NO: 22, the VL-CDR2 comprising the amino acid sequence of SEQ ID NO: 23, and the VL-CDR3 comprising the amino acid sequence of SEQ ID NO: 24.

3. The antibody of claim 1, wherein the antibody comprises a heavy chain (HC) comprising the amino acid sequence set forth in any one of SEQ ID NOs: 99, 131, and 139 and a light chain comprising the amino acid sequence set forth in SEQ ID NO: 160.

4. The antibody of claim 1, wherein the antibody comprises a constant heavy (CH) chain comprising the amino acid sequence set forth in any one of SEQ ID NOs: 35-95, wherein the constant heavy chain comprises CH2 and CH3 domains.

5. The antibody of any one of claim 1, wherein the antibody comprises a heavy chain (HC) comprising the amino acid sequence set forth in any one of SEQ ID NOs: 97-104 and 106-159 and a light chain comprising the amino acid sequence set forth in SEQ ID NO: 160.

6. A composition comprising the antibody of claim 1 and a pharmaceutically acceptable carrier.

7. An isolated polynucleotide or polynucleotides encoding the antibody of claim 1.

8. A vector or vectors comprising the polynucleotide or polynucleotides of claim 7.

9. An isolated cell comprising the polynucleotide or polynucleotides of claim 7.

10. A method of making an antibody that specifically binds to Ebola virus, the method comprising:
(a) culturing the cell of claim 9 under conditions that result in the expression of the antibody, and
(b) isolating the antibody.

11. A method of enhancing at least one of the following in a subject in need thereof:
(a) complement deposition;
(b) cellular phagocytosis; and
(c) NK cell activation;
the method comprising administering to the subject, the antibody of claim 1.

12. A method for treating Ebola virus infection comprising administering to a subject in need thereof a composition comprising an effective amount of an isolated monoclonal antibody, wherein the monoclonal antibody has an Fab binding domain that binds to the Ebola virus glycoprotein, and an Fc domain comprising constant heavy (CH)2 and CH3 domains;
wherein the Fab binding domain comprises
(a) a heavy chain variable region (VH) comprising a VH-complementarity determining region (CDR)1, a VH-CDR2, and a VH-CDR3 from the amino acid sequence of SEQ ID NO:25; and
(b) a light chain variable region (VL) comprising a VL-CDR1, a VL-CDR2, and a VL-CDR3 from the amino acid sequence of SEQ ID NO:26; and
and wherein the Fc domain comprises an amino acid sequence with at least 95% sequence identity to the amino acid sequence set forth in any one of SEQ ID NOs: 35-95.

13. The method of claim 12, wherein the VH comprises the VH-CDR1 comprising the amino acid sequence of SEQ ID NO: 19, the VH-CDR2 comprising the amino acid sequence of SEQ ID NO: 20, and the VH-CDR3 comprising the amino acid sequence of SEQ ID NO: 21; and the VL comprises the VL-CDR1 comprising the amino acid sequence of SEQ ID NO: 22, the VL-CDR2 comprising the amino acid sequence of SEQ ID NO: 23, and the VL-CDR3 comprising the amino acid sequence of SEQ ID NO: 24.

14. The method of claim 12, wherein the antibody comprises a constant heavy (CH) chain with the amino acid sequence set forth in any one of SEQ ID NOs: 35-95, wherein the constant heavy chain comprises CH2, and CH3 domains.

15. The method of claim 14, wherein the antibody comprises a heavy chain (HC) comprising the amino acid sequence set forth in any one of SEQ ID NOs: 97-104 and 106-159 and a light chain comprising the amino acid sequence set forth in SEQ ID NO: 160.

16. The method of claim 15, wherein the antibody comprises a heavy chain (HC) comprising the amino acid sequence set forth in any one of SEQ ID NOs: 99, 131, and 139 and a light chain comprising the amino acid sequence set forth in SEQ ID NO: 160.

17. (canceled)

18. The method of claim 12, further comprising administering a therapeutic agent.

19. The method of claim 18, wherein the therapeutic agent is one or more of interferon alpha, atoltivimab, maftivimab, odesivimab-ebgn, and ansuvimab-zykl.

20. (canceled)

21. A method for producing a monoclonal antibody with a functional profile directed against a pathogen of interest; said method comprising the steps of:
a) generating a library of IgG1 Fc domains, each comprising a different Fc mutation, thereby generating Fc variants; and
b) generating plasmids encoding each of the Fc variants linked to an Fab binding domain, wherein the Fab binding domain comprises variable heavy and light chains of an antibody that is reactive against the pathogen of interest, thereby forming a Fab-Fc variant; and
c) expressing the Fab-Fc variants from the plasmids; and
d) determining the functional profile of each Fab-Fc variant, thereby producing a monoclonal antibody with a profile of functional activity directed against a pathogen of interest.

22. The method of claim 21, wherein the functional profile comprises determining the level of at least one of phagocytosis of monocytes and/or neutrophils; complement deposition; NK cell degranulation; NK cell secretion of cytokine IFNγ and chemokine MIP-1β and expression of membrane protein CD107a; neutralizing activity; and FcyR binding.

23.-34. (canceled)
